(19) United States
(12) Patent Application Publication
(10) Pub. No.: US 2024/0011070 A1
(43) Pub. Date: Jan. 11, 2024

(54) METHODS AND COMPOSITIONS FOR PROTEASE REPORTER ASSAYS AND MODULATORS

(71) Applicant: 712 North Inc., Oakland, CA (US)

(72) Inventor: Marcel V. Alavi Khorassani Moghadam, Berkeley, CA (US)

(73) Assignee: 712 North Inc., Oakland, CA (US)

(21) Appl. No.: 18/019,733

(22) PCT Filed: Jul. 27, 2021
(86) PCT No.: PCT/US2021/043266

§ 371 Date: Feb. 3, 2023

Related U.S. Application Data
(60) Provisional application No. 63/209,138, filed on Jun. 10, 2021.
(60) Provisional application No. 63/061,156, filed on Aug. 4, 2020.

(51) Int. Cl. C12Q 1/37; C12Q 1/66; C12N 15/63

(52) U.S. Cl. CPC C12Q 1/37; C12Q 1/66; C12N 15/63; G01N 2800/50

(57) ABSTRACT

The present invention relates to a reporter for measuring OMA1 protease activity comprising a targeting sequence and a signal producing domain, wherein the targeting sequence is also the sequence recognized by OMA1.

### CROSS-REFERENCE TO RELATED APPLICATIONS

This application claims the benefit under 35 U.S.C. 119(e) of U.S. Provisional Applications No. 63/061,156 filed on Aug. 4, 2020 and U.S. Provisional Applications No. 63/209,138 filed on Jun. 10, 2021, which are incorporated herein by reference in their entirety.

### FEDERAL FUNDING

This invention was made with government support under Grant No. 1R43AG063642-01 awarded by the National Institutes of Health. The federal government may have certain rights in this invention.

This application makes references to disclosures provided by the inventor in a patent of his published as U.S. Pat. No. 10,906,931B2 with the title “Methods for treating diseases related to mitochondrial stress”.

The foregoing patent, and all documents cited therein or during the prosecution (“patent's cited documents”) and all documents cited or referenced in the patent's cited documents, and all documents cited or referenced herein (“herein cited documents”), and all documents cited or referenced in herein cited documents, together with any manufacturer's instructions, descriptions, product specifications, FDA labels and product sheets for any products mentioned herein or in any document incorporated by reference herein, are hereby incorporated herein by reference, and may be employed in the practice of the invention. Citation or identification of any document in this application is not an admission that such document is available as prior art to the present invention.

### REFERENCE TO SEQUENCE LISTING

This application is filed with a sequence listing in electronic form. The sequence listing is provided as a file, which is 84,952 bytes in size, created on Jun. 10, 2021 with a title of Luke_Seq_ST25.txt The information in the sequence listing in electronic form is incorporated by reference herein in its entirety.

### REFERENCE TO A DEPOSIT OF BIOLOGICAL MATERIAL

This application contains a reference to a deposit of biological material which has been made under the Budapest Treaty on Apr. 7, 2021 at the ATCC Patent Depository, 10801 University Boulevard, Manassas, Virginia 20110, USA and was assigned accession number PTA-127022, which deposit is incorporated herein by reference.

### FIELD

The field of this invention relates to novel polypeptides, genetic sequences as well as methods for combining genetic sequences, such that the encoded polypeptide has certain activities, which are useful inter alia for identifying, selecting, or improving compounds with OMA1 and/or OPA1 modulator properties for the treatment of a subject in need of such treatment. The present invention also relates to such compounds, pharmaceutical compositions comprising these compounds, chemical processes for preparation of these compounds, and their use as pharmacological tools or in the treatment of diseases linked to OMA1 and/or OPA1 in cells, animals and in particular humans. The present disclosure provides novel reporter, which are useful for the identification of compounds with OMA1 and/or OPA1 modulatory properties, methods for the design of such reporter, and methods of their use in drug screening assays. Herein is also disclosed the use of a reporter to assess mitochondrial toxicity of a compound and/or predict adverse events of a compound in a subject.

### SUMMARY OF THE INVENTION

The present invention provides novel reporter-genes that upon expression in a host enable the in vivo measurement of OMA1 protease activity. These synthetic genes are built in a modular fashion and operatively combine separate elements: (a) a targeting signal; (b) an entity or fragment “N” of an enzymatic moiety or protein domain; (c) an entity or fragment “C” of an enzymatic moiety or protein domain that is corresponding to N; and (d) a hydrolysable sequence-motif that may be recognized by the OMA1 protease, whereby the complementation of N and C can produce a signal that can be measured. The present invention provides further synthetic mitochondrial import signals that can target a polypeptide or reporter to the mitochondrial inner membrane.

The present invention solves the problem of a specific OMA1 protease assay by targeting said reporter to the mitochondrial inner membrane, where it is recognized and its activity altered by the OMA1 protease. These novel and innovative target-based cellular in vivo protease assays show an inverse correlation with OMA1 protease activity and thereby overcome current limitations probing for OMA1 activity. The disclosed assays can be used in vivo, they are robust and suitable for high-throughput drug screening as demonstrated by the inventor.

The OMA1 protease is a highly desirable drug target with many disease implications supported by epidemiological and genetic data from humans and animal disease models. Examples of such diseases were disclosed in a non-limiting list in U.S. Pat. No. 10,906,931B2 by the inventor. The herein disclosed reporter was used for high-throughput drug screening. The inventor has described the screening campaigns in the herein provided examples and in a manuscript entitled “Extensive OMA1 protease activation by kinase inhibitors”. Said manuscript with all the data is hereby incorporated herein in its entirety.

Herein disclosed are drugs with OMA1 modulatory properties. These drugs are approved by the regulatory authorities for use in humans for treatment of certain malignant diseases. With the teachings provided herein, the teachings provided in U.S. Pat. No. 10,906,931B2 and the other incorporated documents, a skilled artisan is readily enabled to use the herein disclosed drugs for treatment of a subject with a disease or a pathological condition who would benefit from such treatment. It is to be understood that a skilled artisan, conversely, is readily enabled to also identify those subjects who would not benefit from a treatment with a drug with OMA1 modulatory properties or when to stop a treatment with a drug with OMA1 modulatory properties due to an increased risk of adverse events.

### BRIEF DESCRIPTION OF THE DRAWINGS

FIG. 1 illustrates a generic OMA1 reporter of present invention and its general function.

FIG. 1A illustrates a gene encoding different elements of an OMA1 reporter.

FIG. 1B shows a Western blot of a reporter, which can be hydrolyzed by OMA1.

FIG. 1C shows an OMA1 reporter assay. The OMA1 reporter produces a signal which is inversely correlated to OMA1 protease activity. (Mean±SD; n=80 & 48, respectively; T-Test: p<0.001).

FIG. 2 illustrates a vector suitable for the expression of a reporter of present invention.

FIG. 3 compares two reporter Rep #01 and Rep #15 expressed in Hek293T cells, which were incubated for 30 minutes without or with 10 μM CCCP before measuring the signal. Rep #15 shows a much better performance than Rep #01. (Mean±SD; n=4; note the two different scales of the Y-axes).

FIG. 4 compares Rep #01 and Rep #15 in Western blots and establishes OMA1 specificity.

FIG. 4A shows a Western with reporter-specific antibody (NanoLuc) labeling of 25 μg reporter-cell lysate separated on a 12% PAGE. Rep #15 appears much more abundant than Rep #01.

FIG. 4B shows a Rep #15 assay. To establish OMA1 specificity, reporter cells were preincubated for an hour with 500 μM phenanthroline (an OMA1 inhibitor) before 10 μM CCCP was added for another 30 minutes. (Mean±SD; n=4; 1-way ANOVA: p<0.001).

FIG. 5 illustrates the temporal performance of Rep #01 and Rep #15 assays.

FIG. 5A shows Rep #01 signals at indicated times after addition of luciferase substrate at t=0. Rep #01 Hek293T cells were exposed for 30 minutes to media without or with 10 μM CCCP prior the measurements. (Mean±SD; n=4).

FIG. 5B shows Rep #15 signals at indicated times after addition of luciferase substrate at t=0. Rep #15 Hek293T cells were exposed for 30 minutes to media without or with 10 μM CCCP prior the measurements. (Mean±SD; n=4).

FIG. 5C provides the calculated Z′ values for Rep #01 and Rep #15 at the different times.

FIG. 6 compares Rep #15 with two different recognition sequences. Rep #15-S1 incorporates the OPA1 S1 cleavage site. In a TEV cleavage site replaces the S1 site in Rep #15-TEV as recognition sequences. Both reporters were stably expressed in Neuro2A cells, which were exposed to increasing valinomycin concentrations for 30 minutes before measuring the signals. Both reporter show comparable valinomycin dose-response relationships. (Mean±SD; n=2).

FIG. 7 illustrates different reporter and how they combine different functional elements.

FIG. 8 compares different Hek293T reporter cells without and with CCCP in Western blots.

FIG. 8A shows hydrolysis of Rep #01 (arrows) in CCCP-treated cells. The cleavage product was also recognized by the antibody (asterisk).

FIG. 8B shows Rep #04 (arrow) in untreated and CCCP-treated cells.

FIG. 8C shows Rep #08 (arrow) in untreated and CCCP-treated cells.

FIG. 8D shows Rep #10 (arrows) in untreated and CCCP-treated cells.

FIG. 8E shows hydrolysis of Rep #15 (arrows) in CCCP-treated cells. The cleavage product was also recognized by the antibody (asterisk).

FIG. 9 provides use examples of how to assess mitochondrial toxicity with Rep #01 or Rep #15.

FIG. 9A shows significantly reduced bioluminescence of Rep #01 cells exposed for 30 minutes to the denoted molecules. (Mean±SD; n=4; 1-way ANOVA: p<0.001).

FIG. 9B shows significantly reduced bioluminescence of Rep #15 cells exposed for 30 minutes to the denoted molecules. (Mean±SD; n=4; 1-way ANOVA: p<0.001).

FIG. 10 illustrates a dose-response relationship of tipranavir and of kavain in Rep #15 assays.

FIG. 10A shows Hek293T Rep #15 cells exposed to increasing concentrations of tipranavir for 60 minutes before measuring bioluminescence. (Mean±SD; n=4; EC₅₀: 9 μM).

FIG. 10B shows Hek293T Rep #15/Luke-S1 cells exposed to increasing concentrations of kavain for 60 minutes before measuring bioluminescence. (Mean±SD; n=2; EC₅₀: 14 μM).

FIG. 11 illustrates the characteristics of a luciferase-based OMA1 protease reporter.

FIG. 11A shows the design of the Luke-S1 called reporter (i.e. Rep #15) and the Luke-TEV reporter, in which the S1 site is replaced with a TEV site.

FIG. 11B shows the enzyme kinetics for Luke-S1 in stably transfected Hek293T cells.

FIG. 11C shows the enzyme kinetics for Luke-TEV in stably transfected Hek293T cells.

FIG. 11D shows the enzyme kinetics for the unmodified, native luciferase ‘Luke’.

FIG. 12 shows Luke-S1 and Luke-TEV are hydrolyzed under conditions OPA1 is hydrolyzed and confirms their mitochondrial translocation.

FIG. 12A illustrates CCCP-dependent OPA1 hydrolysis in Hek293T cells after 30 minutes of exposure. 3 μM CCCP led to complete L-OPA1 proteolysis in Western blots.

FIG. 12B illustrates valinomycin-dependent OPA1 hydrolysis in Hek293T cells after 30 minutes of exposure. 0.1 μM valinomycin led to complete L-OPA1 proteolysis in Western blots.

FIG. 12C shows that 3 μM CCCP and 0.1 μM valinomycin (vine) induced Luke-S1 and Luke-TEV cleavage in Hek293T reporter cells.

FIG. 12D shows that Luke-S1 and Luke-TEV comigrated together with OPA1 and OMA1 in mitochondria-enriched fractions (P) in Western blots upon cell fractionation by differential centrifugation. P, pellet; S, supernatant; Vmc, valinomycin.

FIG. 13 shows non-limiting and merely illustrative examples of a response of different Hek293T reporter cells to CCCP and valinomycin after 30 minutes of incubation.

FIG. 13A shows a CCCP dose-response curve for Luke-S1 cells.

FIG. 13B shows a valinomycin dose-response curve for Luke-S1 cells.

FIG. 13C shows a CCCP dose-response curve for Luke-TEV cells.

FIG. 13D shows a valinomycin dose-response curve for Luke-TEV cells.

FIG. 13E shows a CCCP dose-response curve for Luke cells.

FIG. 13F shows a valinomycin dose-response curve for Luke cells.

FIG. 14 provides additional data on the specificity and the dynamic behavior of the reporter.

FIG. 14A confirms that OMA1 knock-down can prevent valinomycin-induced signal reduction in Luke-S1 assays. Valinomycin (vmc) led to a significant signal reduction in Luke-S1 cells treated with control siRNA but not in Luke-S1 cells treated with OMA1 siRNA. (n=3; 1-way ANOVA: p=0.003).

FIG. 14B shows a Western blot of Luke-S1 cells treated with control siRNA (cntrl.) or OMA1 siRNA labeled with the denoted antibodies. OMA1 levels were reduced by about 70%.

FIG. 14C shows the dynamic behavior of the Luke-S1 reporter. Bioluminescence was recorded over the course of 25 minutes in 20-second intervals immediately after adding luciferase substrate without or with 100 nM valinomycin to the cells. Note, signal decay in this assay is a compound effect of reporter enzyme inactivation and substrate depletion over time.

FIG. 14D shows the dynamic behavior of the Luke-TEV reporter.

FIG. 14E shows the dynamic behavior of the Luke reporter.

FIG. 15 provides a merely illustrative example of a drug screen for OMA1 activators for which Hek293T Luke-S1 cells were exposed to test compounds for 1-2 hours and the signal intensity compared to 100 nM valinomycin-treated cells.

FIG. 15A shows a representative valinomycin-treated Luke-S1 cells in columns #2 and #23 as positive controls.

FIG. 158 compares untreated Luke-S1 cells with valinomycin-treated Luke-S1 cells.

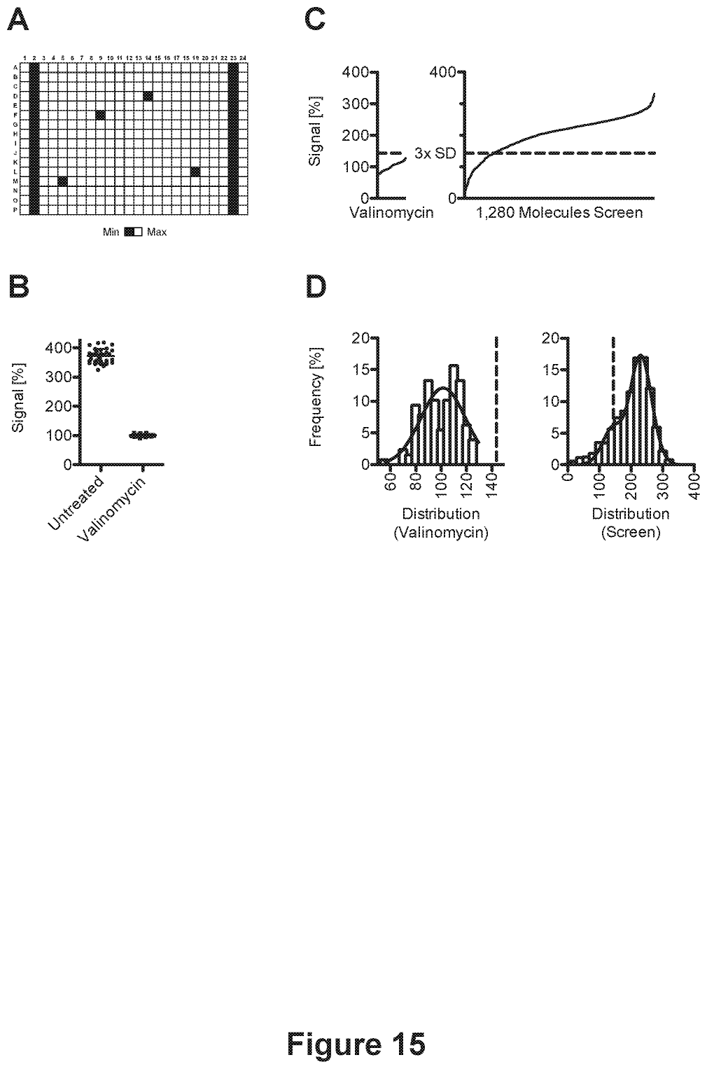

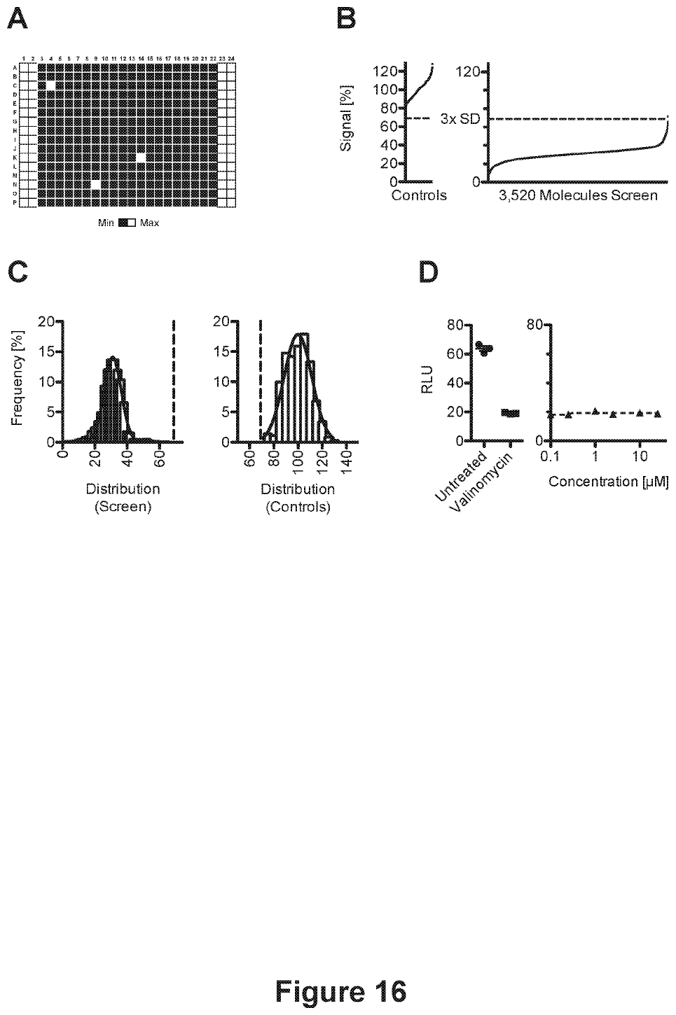

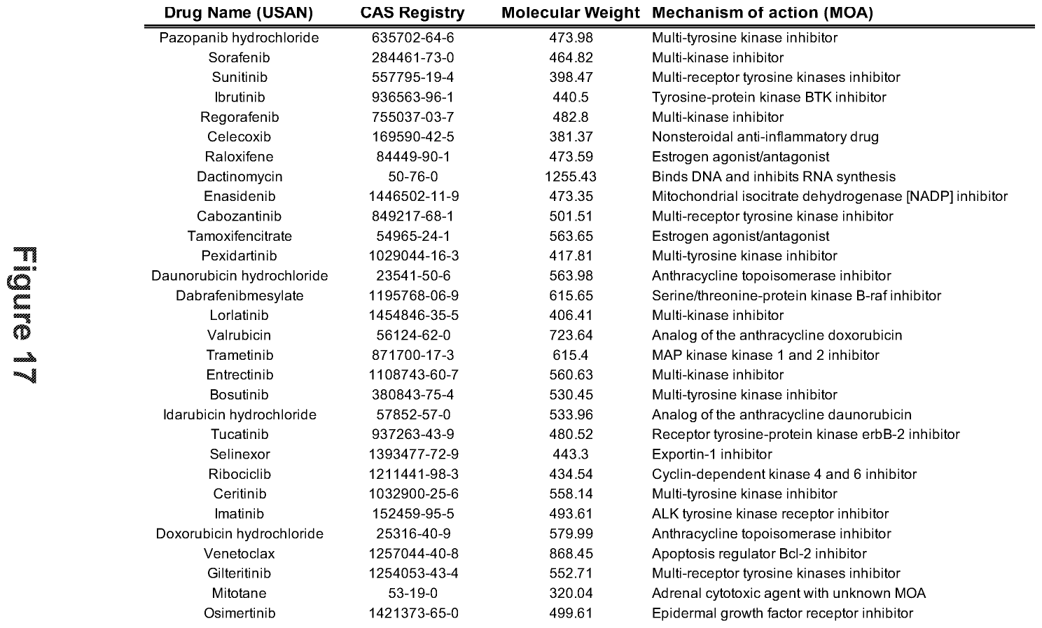

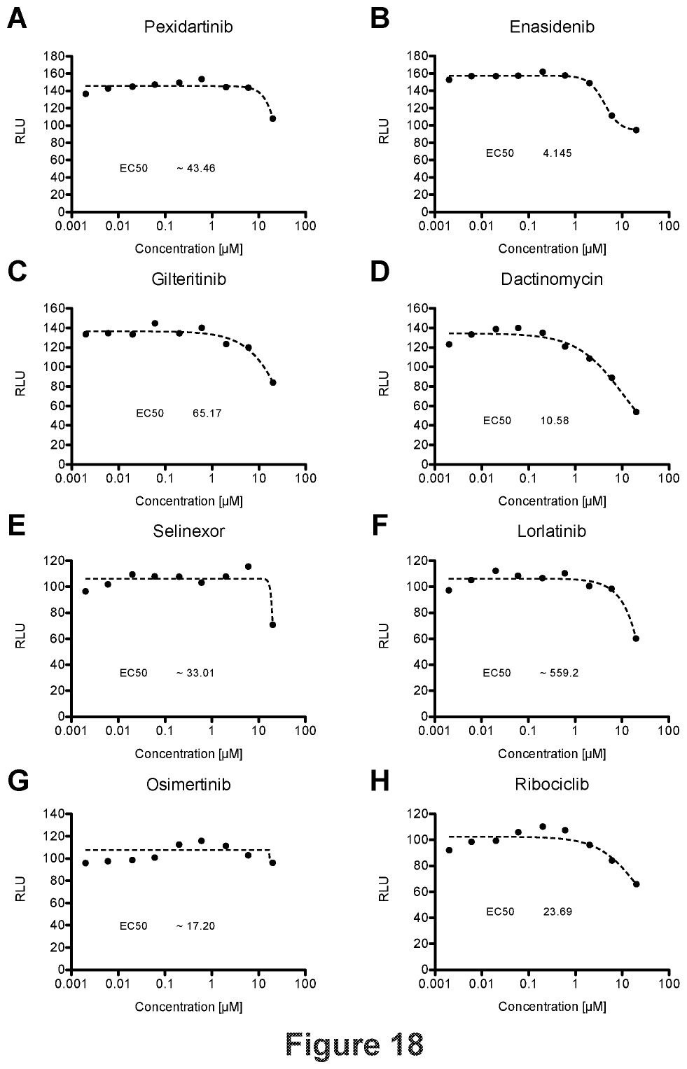

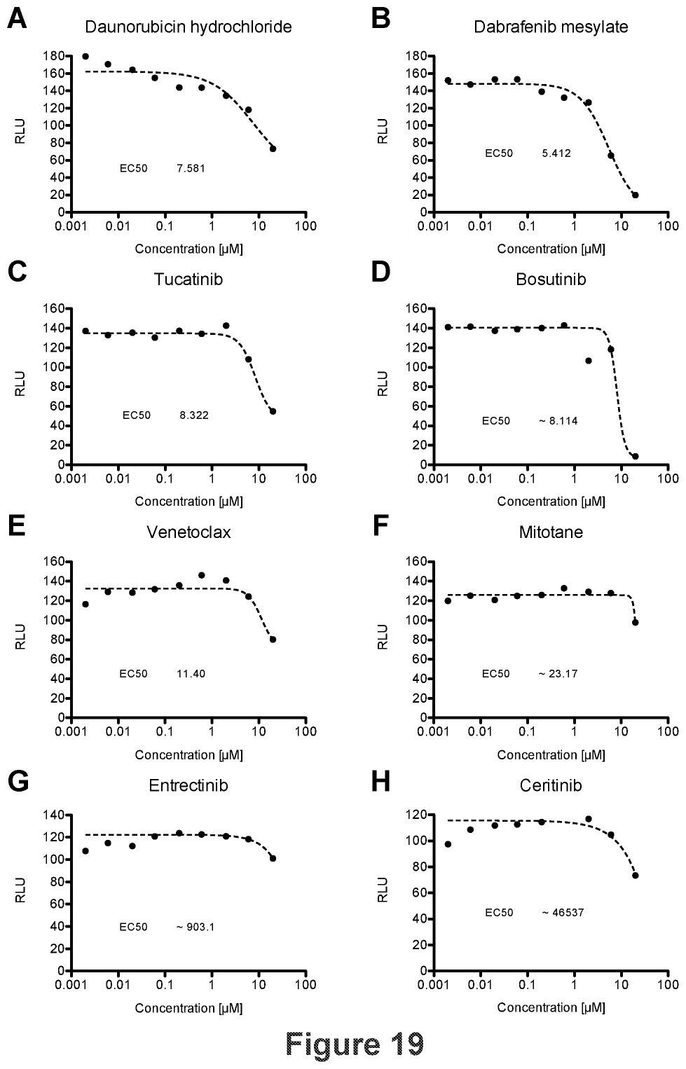

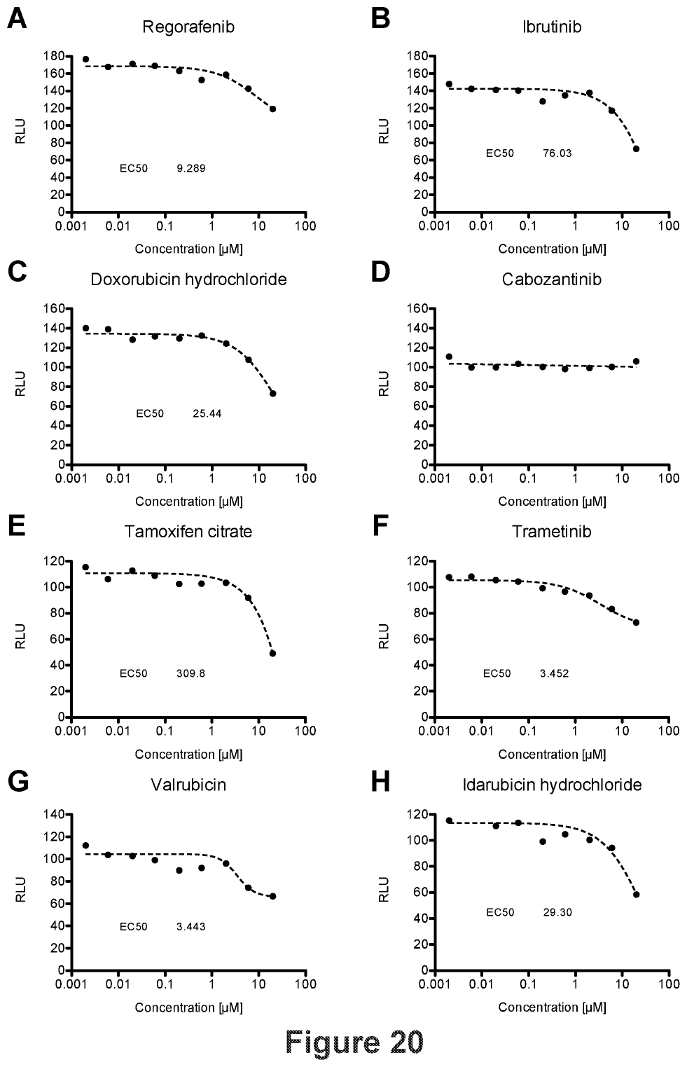

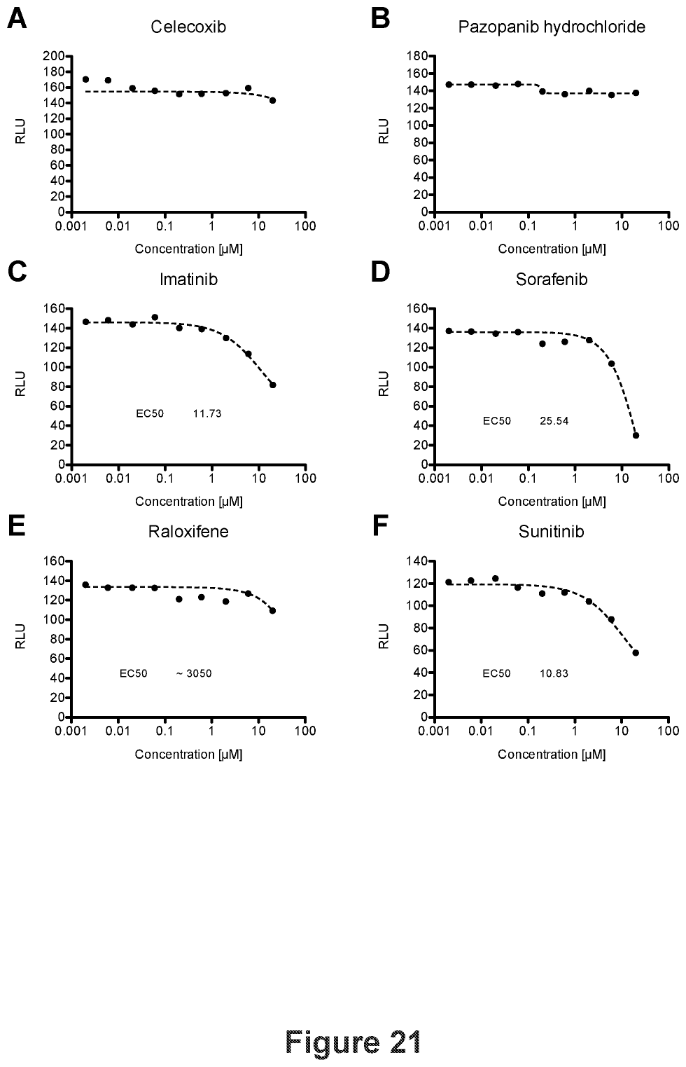

FIG. 15C shows the signal of 1,280 chemically diverse molecules ranked by intensity; the signal was normalized to the mean of 128 valinomycin-treated samples, which was defined as 100%. Molecules with a signal within 3 standard deviations (SD, dotted line) of valinomycin-treated cells were considered potential OMA1 activators.

FIG. 15D shows the signal distribution of the 128 valinomycin-treated samples and the 1,280 test molecules with the hit-threshold (3×SD) depicted as dotted line.

FIG. 16 provides a merely illustrative example of a drug screen for OMA1 inhibitors for which Luke-S1 cells were preincubated with test compounds for 60 minutes before adding 100 nM valinomycin for another 30 to 60 minutes. The signal intensity was compared to untreated Luke-S1 cells.

FIG. 16, panel A shows a representative plate with untreated Luke-S1 cells in columns #2 and #23 as positive controls.

FIG. 16, panel B shows the signal of 3,520 chemically diverse molecules ranked by intensity; the signal was normalized to the mean of 352 untreated samples, which was defined as 100%. Molecules with a signal within 3 standard deviations (SD, dotted line) of untreated cells were considered potential OMA1 inhibitors.

FIG. 16, panel C shows the signal distribution of the 352 untreated samples and the 3,520 test molecules with the hit-threshold (3×SD) depicted as dotted line

FIG. 16, panel D shows a 6-point dose-response curve of Luke-S1 cells for the only molecule which crossed the hit-threshold.

FIG. 17 shows a table with FDA-approved drugs that significantly reduced Luke-S1 bioluminescence by more than 37.5% in a screen of 166 cancer drugs (10 μM for 1 hour).

FIG. 18 shows a non-limiting and merely illustrative example of a dose-response curve for Hek293T Luke-S1 cells exposed to an FDA-approved drug for 1 hour.

FIG. 18A shows a Pexidartinib dose-response curve for Luke-S1 cells.

FIG. 18B shows an Enasidenib dose-response curve for Luke-S1 cells.

FIG. 18C shows a Gilteritinib dose-response curve for Luke-S1 cells.

FIG. 18D shows a Dactinomycin dose-response curve for Luke-S1 cells.

FIG. 18E shows a Selinexor dose-response curve for Luke-S1 cells.

FIG. 18F shows a Lorlatinib close-response curve for Luke-S1 cells.

FIG. 18G shows an Osimertinib dose-response curve for Luke-S1 cells.

FIG. 18H shows a Ribociclib dose-response curve for Luke-S1 cells.

FIG. 19 shows a non-limiting and merely illustrative example of a dose-response curve for Hek293T Luke-S1 cells exposed to an FDA-approved drug for 1 hour.

FIG. 19A shows a Daunorubicin hydrochloride dose-response curve for Luke-S1 cells.

FIG. 19B shows a Dabrafenib mesylate dose-response curve for Luke-S1 cells.

FIG. 19C shows a Tucatinib dose-response curve for Luke-S1 cells.

FIG. 19D shows a Bosutinib close-response curve for Luke-S1 cells.

FIG. 19E shows a Venetoclax dose-response curve for Luke-S1 cells.

FIG. 19F shows a Mitotane dose-response curve for Luke-S1 cells.

FIG. 19G shows an Entrectinib dose-response curve for Luke-S1 cells.

FIG. 19H shows a Ceritinib dose-response curve for Luke-S1 cells.

FIG. 20 shows a non-limiting and merely illustrative example of a dose-response curve for Hek293T Luke-S1 cells exposed to an FDA-approved drug for 1 hour.

FIG. 20A shows a Regorafenib dose-response curve for Luke-S1 cells.

FIG. 20B shows an Ibrutinib dose-response curve for Luke-S1 cells.

FIG. 20C shows a Doxorubicin hydrochloride dose-response curve for Luke-S1 cells.

FIG. 20D shows a Cabozantinib dose-response curve for Luke-S1 cells.

FIG. 20E shows a Tamoxifen citrate dose-response curve for Luke-S1 cells.

FIG. 20F shows a Trametinib dose-response curve for Luke-S1 cells.

FIG. 20G shows a Valrubicin close-response curve for Luke-S1 cells.

FIG. 20H shows an Idarubicin hydrochloride dose-response curve for Luke-S1 cells.

FIG. 21 shows a non-limiting and merely illustrative example of a dose-response curve for Hek293T Luke-S1 cells exposed to an FDA-approved drug for 1 hour.

FIG. 21A shows a Celecoxib close-response curve for Luke-S1 cells.

FIG. 21B shows a Pazopanib hydrochloride dose-response curve for Luke-S1 cells.

FIG. 21C shows an Imatinib dose-response curve for Luke-S1 cells.

FIG. 21D shows a Sorafenib dose-response curve for Luke-S1 cells.

FIG. 21E shows a Raloxifene dose-response curve for Luke-S1 cells.

FIG. 21F shows a Sunitinib dose-response curve for Luke-S1 cells.

### DETAILED DESCRIPTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting. The preferred materials and methods are described herein, although a number of methods and materials similar or equivalent to those described herein can be used in the practice of the present invention.

The compositions and methods provided herein relating to reporters and assays are useful in a variety of fields including basic research, medical research, molecular diagnostics, etc., although the reporters and assays described herein are not limited to any particular applications, and any useful application should be viewed as being within the scope of the present invention, drug development is an example for the utility of present invention.

Mitochondria are dynamic cell organelles forming networks of interconnected tubules, which maintain homeostasis by constantly fusing and dividing. Fragmented mitochondria thereby are more susceptible to apoptotic cell death, while fused mitochondria show stress resistance.

OPA1 is an essential fusion protein, which exists, as matter of principal, in two forms, large L-OPA1 isoforms, which are anchored to the mitochondrial inner membrane, and small S-OPA1 isoforms, which have no transmembrane domain. S-OPA1 is derived from L-OPA1 by proteolytic cleavage by the OMA1 protease and the YME1L1 protease, which is also known as i-AAA protease. It is well accepted that L-OPA1 is necessary for mitochondrial fusion. S-OPA1 on the other hand is believed to function in mitochondrial outer membrane permeabilization and cytochrome c release, because there is a high correlation of L-OPA1 cleavage and programmed cell death.

OMA1 is a mitochondrial inner membrane protease of the MEROPS M48-family of zinc-metalloendopeptidases (see Rawlings N D, et al., Nucleic Acids Res. (2014) 42(Database issue):D503-D509), OMA1 cleaves substrates involved in signaling pathways, such as DELE1, which signals to the integrated stress response. The OMA1 protease cleaves the OPA1 fusion protein and thereby generates S-OPA1 under conditions broadly defined as cell-stress, whereby OMA1 activation facilitates outer membrane permeabilization and cytochrome c release ultimately resulting in apoptotic cell death.

It is known in the arts that mitochondrial dysfunction (or a corresponding mitochondrial disease or disorder) is correlated with decrease of L-OPA1. However, it is also within the context of the present invention that additional, possibly existing OPA1 isoforms or other proteins, such as DELE1, PGAM5 and PINK1, may be altered.

In this context, it is to be understood that the OPA1 isoforms merely serve as proxy for protease activity, in particular for OMA1 protease activity. Therefore, the present invention is not limited to the modulation of OPA1 isoforms but encompasses also any and every other proxy for OMA1 activity, including other OMA1 substrates, such as DELE1, PGAM5, or PINK1. A person skilled in the art is also readily in a position to deduce further amino acid stretches/peptides that are (artificial) OMA1 substrates, which are also within the scope of present invention.

Conversely, compounds of present disclosure may modulate the ratio of OPA1 isoforms by direct or indirect interaction with the OMA1 protease, for example by interacting with a protein complex comprising OMA1, or by interacting with other proteases, which may cleave OPA1 and/or OMA1, such as the i-AAA protease, or by interacting with OMA1-regulating enzymes, such as the m-AAA protease or prohibitin (see also Alavi M. V. Biochim Biophys Acta Proteins Proteom. 2020 Oct. 29:140558.)

The OMA1 protease is a highly desirable drug target with many disease implications supported by epidemiological and genetic data from humans and animal disease models. And yet, there are still no specific OMA1 inhibitors or activators available, let alone any drugs that target the OMA1 protease, with exception of the disclosed drugs by the inventor herein and in U.S. Pat. No. 10,906,931B2.

The problem with the development of OMA1 modulators is two-fold (=double-whammy): (1) the OMA1 protease is rather promiscuous in its substrate recognition and there is no clear consensus motif known for the cleavage site. This makes it nearly impossible to rationally design OMA1 inhibitors based on the substrate recognition site; (2) the OMA1 protease undergoes auto-proteolysis once it is activated, which makes it nearly impossible to isolate the functional protein for in vitro enzymatic assays. As a result, there are no specific OMA1 modulators available.

Most protease assays are based on fluorescence resonance energy transfer (FRET) from a donor fluorophore to a quencher placed at opposite ends of a short peptide chain containing the potential cleavage site (see Knight C G, Methods in Enzymol. (1995) 248:18-34.) Proteolysis separates the fluorophore and quencher, resulting in increased intensity in the emission of the donor fluorophore.

This general principal can also be used for the design of an OMA1 protease assay using the “S1” protease cleavage site of the rat OPA1 protein, which has the following amino acid sequence: Ala-Phe-Arg-/-Ala-Thr-Asp-His-Gly (A-F-R-/-A-T-D-H-G; where “-/-” indicates the scissile bond cleaved by OMA1). The Hydrolysis of the scissile bond between the donor/acceptor pair of the FRET peptide generates fluorescence that allegedly permits the measurement of OMA1 protease activity.

Such a FRET-based assay is known in the art (see for example U.S. Pat. No. 10,739,331B2). However, such FRET-based assays are useless for the measurement of OMA1 activity. The problem is: the linker-sequence Ala-Phe-Arg-Ala-Thr-Asp-His-Gly can be recognized by a number of proteases, including Endoproteinase Arg-C, Endoproteinase Asp-N, Chymotrypsin, Clostripain, Pepsin, Proteinase K, Thermolysin, and Trypsin. As a result, FRET-based assays show only little to no specificity when used with crude cell fractions. A highly purified functional OMA1 protease is the sine qua non to achieve specificity in such in vitro assays. As already mentioned above, it is inherently challenging to isolate functional OMA1 enzyme for in vitro assays because the OMA1 protease digests itself upon activation (see Baker M J et al., EMBO Journal (2014) 33(6):578-593; Zhang K et al., EMBO Reports (2014) 15(5):576-585.). Because of the specific nature of the fluorophore and the quencher, FRET-based assays are also restricted to in vitro use. The fluorophores cannot be genetically encoded and therefore not be used in vivo, which would increase specificity for example by confining the reporter to mitochondria.

Another limitation of the FRET-based in vitro assays is that they produce a signal upon OMA1 activation. This means more OMA1 activity results in more fluorescence. In other words, there is a positive correlation between OMA1 protease activity and the detected signal. This makes these kinds of assays less desirable for drug screening campaigns, because they have a higher false-hit rate when screening for OMA1 inhibitors. All compounds that interfere with the signal would be considered potential OMA1 inhibitors irrespective if these hit compounds inhibit OMA1 or just quench the FRET donor. Furthermore, in vitro assays have only limited predictive value of the in vivo pharmacodynamics of a particular hit compound, because for instance cell permeability is not accounted for in such assays.

The present invention solves the problem of a specific OMA1 protease assay by targeting a reporter or an enzyme to the mitochondrial inner membrane, where it is recognized and its activity altered by the OMA1 protease. These novel and innovative target-based cellular in vivo protease assays show an inverse correlation with OMA1 protease activity and thereby overcome current limitations probing for OMA1 activity. The disclosed assays can be used in vivo, they are robust and suitable for high-throughput drug screening as demonstrated in the examples.

Targeting of an OMA1 reporter to mitochondria may seem obvious. However, it has not yet been accomplished, though many skilled artisans may have tried it without success. The problem is that the OPA1 import sequence, which would be the obvious thing to try, does not lend itself for the translocation of an OMA1 reporter to the mitochondrial inner membrane. Only serendipity and a certain flash of genius—if I may add—led to the discovery of the OMA1 reporter constructs of present invention. The reporter of present invention are characterized by the absence [emphasis added] of the mitochondrial import sequence of OPA1, which is against all teachings in the art (see for example U.S. Pat. No. 10,739,331B2). Unexpectedly, the reporter translocated into the mitochondria inner membrane with great efficiency, where it was recognized and cleaved by the OMA1 protease, when the first 80 to 90 amino acids of OPA1's amino-terminus were deleted. Eureka!

The present invention provides novel reporter-genes that upon expression in a host enable the in vivo measurement of OMA1 protease activity. These synthetic genes are built in a modular fashion and operatively combine separate elements: (a) a targeting signal; (b) an entity or fragment “N” of an enzymatic moiety or protein domain; (c) an entity or fragment “C” of an enzymatic moiety or protein domain that is corresponding to N; and (d) a hydrolysable sequence-motif that may be recognized by the OMA1 protease, whereby the complementation of N and C can produce a signal that can be measured (see also Figure). The present invention provides further synthetic mitochondrial import signals that can target a polypeptide or reporter to the mitochondrial inner membrane.

Embodiments described herein may find use in drug screening anchor drug development. For example, the interaction of a small molecule drug or an entire library of small molecules with a target protein of interest (e.g., therapeutic target) is monitored under one or more relevant conditions (e.g., physiological conditions, disease conditions, etc.). In other embodiments, the ability of a small molecule drug or an entire library of small molecules to enhance or inhibit the interactions between two entities is assayed. In some embodiments, drug screening applications are carried out in a high through-put format to allow for the detection of the binding of tens of thousands of different molecules to a target, or to test the effect of those molecules on the binding of other entities.

In some embodiments, the present invention provides the detection of molecular interactions in living organisms (e.g., bacteria, yeast, eukaryotes, mammals, primates, human, etc.) and/or cells. In some embodiments, fusion proteins comprising signal and interaction (target) polypeptides are co-expressed in the cell or whole organism, and signal is detected and correlated to the formation of the interaction complex. In some embodiments, cells are transiently and/or stably transformed or transfected with vector(s) coding for non-luminescent element(s), interaction element(s), fusion proteins (e.g., comprising a signal and interaction element), etc. In some embodiments, transgenic organisms are generated that code for the necessary reporter for carrying out the assays described herein. In other embodiments, vectors are injected into whole organisms. In some embodiments, a transgenic animal or cell (e.g., expressing a reporter) is used to monitor or measure mitochondrial toxicity of a small molecule or a biologic.

In one particular embodiment of the invention, the reporter-gene #15 combined (a) a synthetic polypeptide sufficient and necessary for mitochondrial import with (b) the N-terminal domain and (c) the C-terminal domain of the NanoLuc luciferase enzyme that when combined are capable of emitting a light signal, and (d) OPA1's exon 5, which encodes a peptide that can be recognized by the OMA1 protease. OPA1's exon 5 is thereby positioned within the permutated NanoLuc enzyme in such a way that it operatively links the C-terminal domain with the N-terminal domain (see also U.S. Pat. Nos. 9,757,478B2, 10,107,800B2, 9,339,561B2 and 10,077,433B2). OMA1 activation separates both domains and thereby deactivates the NanoLuc enzyme. In a non-limiting example, reporter-gene #15 was expressed in Hek293T cells under the control of a CMV promotor and shown to work as intended.

In another embodiment of the invention, the reporter-gene #01 combined (a) a portion of OPA1's amino-terminal domain sufficient for mitochondrial import with (b) the NanoLuc C-terminus, (c) OPA1's exon 5, and (d) the NanoLuc N-terminus. Again, exon 5 operatively connected the permutated NanoLuc sequences in such a way that OMA1 activation deactivated the NanoLuc luciferase. In a non-limiting example, reporter-gene #01 was transiently expressed in Hek293T cells under the control of a CMV promotor among others and shown to work as intended.

The present disclosure also provides methods of use of the reporter-genes in cellular assays useful for the screening for potential OMA1 modulators. In a non-limiting example, reporter #15 was used to screen for potential OMA1 inhibitors. To this end, reporter #15 was transiently expressed in Hek293T cells, which produced a robust bioluminescence signal. The OMA 1 enzyme is dormant under physiological cell culture conditions in these cells, but can be readily activated by the addition of CCCP (carbonyl cyanide m-chlorophenyl hydrazone) to the cell-culture medium. Incubation of the transfected cells for 30 minutes in cell-culture medium with 10 μM CCCP resulted in a significantly reduced or substantially absent luciferase activity compared to untreated control cells. The OMA1 inhibitor phenanthroline antagonized the effect that CCCP had on the cells. Preincubation with 500 μM phenanthroline for 1 hour prior to the CCCP-treatment prevented reporter #15 deactivation and maintained the bioluminescence signal. This demonstrates that the disclosed assay is useful for the screening of OMA1 inhibitors.

In addition, the present invention provides methods of testing compounds for potential mitochondrial toxicity. Certain drugs show mitochondrial toxicity, which can result in unwanted side-effects in patients thus limiting their usefulness. It is known that cytotoxic drugs, such as sorafenib, can act through the OMA1-pathway (Zhao X, et al., Laboratory Investigation (2013) 93(1):8-19). In a non-limiting example, sorafenib deactivated reporter #15, which resulted in a significantly reduced signal in the assay, which was comparable to the effects observed with CCCP. Sorafenib had comparable effects on assays performed with reporter #01 as well.

Also other drugs intended for different indications and with known mitochondrial toxicity can activate the OMA1-pathway. Mitochondrial toxicity can ultimately lead to complications in patients. Tipranavir is known in the arts to cause mitochondrial toxicity, which is also listed as an adverse reaction on the Aptivus (tipranavir) FDA label. In another non-limiting example, tipranavir deactivated reporter #15 and reporter #01 in the present assays, thereby indicating mitochondrial toxicity. This provides proof-of-principal of the utility of the assays for the identification of potentially mitochondrial toxins. Additional examples are provided in example 6 and in the claims.

Provided herein are also methods of use of the assays in a scalable micro-titer format suitable for high-throughput screening of compound libraries. In another nonlimiting example, we demonstrate that our assays are robust and amendable for high-throughput screening of compound libraries.

Herein disclosed are also drugs with OMA1 modulatory properties. These drugs are approved by the regulatory authorities for use in humans for treatment of certain malignant diseases and were identified with the OMA1 assays of present invention. The chemical synthesis of the pharmaceutically active ingredients of the drugs of present invention and processes for the preparation of a pharmaceutical composition comprising said pharmaceutically active ingredients are well known in the arts. The FDA label of these drugs and each, every and all other drugs mentioned herein are hereby incorporated herein by reference and may be used in practice of the invention. With the teachings provided herein, the teachings provided in the incorporated documents and in U.S. Pat. No. 10,906,931B2, a skilled artisan is readily enabled to use the herein disclosed drugs for treatment of a subject with a disease inter alia characterized by altered OMA1 levels or activity. It is to be understood that a skilled artisan, conversely, is readily enabled with present disclosure and the provided examples to also identify those subjects who would not benefit from a treatment with a drug with OMA1 modulatory properties or when to stop a treatment with a drug with OMA1 modulatory properties due to an increased risk of adverse events. In this context it is clear that the assays of present disclosure are also useful for the design and development of improved drugs or therapies that avoid OMA1 activation thereby limiting adverse side-effects. Furthermore, assays of present disclosure are also useful for the development of drugs that activate OMA1, for example in cancer cells thereby inhibiting tumor growth.

### Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

References to an “aspect”, “one embodiment”, “an embodiment”, “an example embodiment,” etc., indicate that the aspect described may include a particular feature, structure, or characteristic, but every aspect may not necessarily include that particular feature, structure, or characteristic. Moreover, such phrases are not necessarily referring to the same aspect. Further, when a particular feature, structure, or characteristic is described in connection with an aspect, it is submitted that it is within the knowledge of one skilled in the art to affect such feature, structure, or characteristic in connection with other embodiments whether or not explicitly described.

The singular forms “a”, “an”, and “the” as used herein and in the claims include plural reference unless the context dictates otherwise. For example, “a cell” means as well a plurality of cells, and so forth.

The term “and/or” as used in the present specification and in the claims implies that the phrases before and after this term are to be considered either as alternatives or in combination.

As used herein, the term “OPA1” refers to the mitochondrial dynamin-like protein encoded by the OPA1 gene in eukaryotes. OPA1 is defined as broad as possible and shall include all natural and non-natural variants and homologues thereof from any and every species.

As used herein, the term “OMA1” is known in the art and refers to the mitochondrial inner membrane protease encoded by the OMA1 gene in eukaryotes. OMA1 is defined as broad as possible and shall include all natural and non-natural variants and homologues thereof from any and every species.

As used herein, the term “YME1L1,” is known in the art as a component of the mitochondrial inner membrane i-AAA protease and is encoded by the YME1L1 gene in eukaryotes. YME1L1 is defined as broad as possible and shall include all natural and non-natural variants and homologues thereof from any and every species.

The terms “compound”, “Molecule”, “chemical”, “agent”, “reagent”, “Modulator” and the like refer to any substance, chemical, composition or extract that have a positive or negative biological effect on a cell, tissue, body fluid, or within the context of any biological system, or any assay system.

As used herein, the term “nucleic acid molecule” or “polynucleotide” refers to a ribonucleotide or deoxyribonucleotide polymer in either single-stranded or double-stranded form, and, unless specifically indicated otherwise, encompasses polynucleotides containing known analogs of naturally occurring nucleotides that can function in a similar manner as naturally occurring nucleotides. It will be understood that when a nucleic acid molecule is represented by a DNA sequence, this also includes RNA molecules having the corresponding RNA sequence in which “U” (uridine) replaces “T” (thymidine).

As used herein, the term “recombinant nucleic acid molecule” refers to a non-naturally occurring nucleic acid molecule containing two or more linked polynucleotide sequences. A recombinant nucleic acid molecule can be produced by recombination methods, particularly genetic engineering techniques, or can be produced by a chemical synthesis method. A recombinant nucleic acid molecule can encode a fusion protein, for example, a reporter protein of the invention linked to a polypeptide of interest.

As used herein, the term “recombinant host” or “host” refers to a cell that contains a recombinant nucleic acid molecule. As such, a recombinant host cell can express a polypeptide from a “gene” that is not found within the native (non-recombinant) form of the cell. A recombinant host cell may be transiently transfected or stably transformed or transfected with one or multiple vectors coding for recombinant nucleic acid molecule(s) (e.g., a reporter gene). It is understood that a recombinant host can be generated by any means, which may also include methods that are not specifically mentioned herein.

As used herein, a reference to a polynucleotide “encoding” a polypeptide means that, upon transcription of the polynucleotide and translation of the mRNA produced therefrom, a polypeptide is produced. The encoding polynucleotide is considered to include both the coding strand, whose nucleotide sequence is identical to an mRNA, as well as its complementary strand. It will be recognized that such an encoding polynucleotide is considered to include degenerate nucleotide sequences, which encode the same amino acid residues. Nucleotide sequences encoding a polypeptide can include polynucleotides containing introns as well as the encoding exons.

As used herein, the term “expression control sequence” refers to a nucleotide sequence that regulates the transcription or translation of a polynucleotide or the localization of a polypeptide to which it is operatively linked. Expression control sequences are “operatively linked” when the expression control sequence controls or regulates the transcription and, as appropriate, translation of the nucleotide sequence (i.e., a transcription or translation regulatory element, respectively), or localization of an encoded polypeptide to a specific compartment of a cell. Thus, an expression control sequence can be a promoter, enhancer, transcription terminator, a start codon (ATG), a splicing signal for intron excision and maintenance of the correct reading frame, a STOP codon, a ribosome binding site, or a sequence that targets a polypeptide to a particular location, for example, a particular cell compartment,

As used herein, the term “targeting signal” or “targeting peptide” or “targeting sequence” or the like refers to a peptide or polypeptide that can target a polypeptide inter alia to the cytosol, nucleus, plasma membrane, endoplasmic reticulum, mitochondrial outer membrane, mitochondrial inner membrane, mitochondrial intermembrane space or matrix, chloroplast outer or thylakoid membrane, intermembrane space or lumen, medial trans-Golgi cisternae, or a lysosome or endosome. Cell compartmentalization domains are well known in the art and include, for example, a peptide containing amino acid residues 1 to 81 of human type II membrane-anchored protein galactosyltransferase, or amino acid residues 1 to 12 of the presequence of subunit IV of cytochrome c oxidase (see, also, Hancock et al., EMBO J. (1991) 10:4033-4039; Buss et al., Mol. Cell. Biol. (1988) 8:3960-3963; U.S. Pat. No. 5,776,689, which are incorporated herein by reference).

As used herein, the term “mitochondrial targeting signal” or “mitochondrial signaling peptide” or “mitochondrial import sequence” or the like refers to a peptide or polypeptide that can target a polypeptide to the mitochondria. It is to be understood that such a mitochondrial targeting sequence can direct a polypeptide or protein to the mitochondrial outer membrane or the mitochondrial inner membrane or the mitochondrial intermembrane space or the mitochondrial matrix depending on the nature of said mitochondrial targeting sequence. A mitochondrial targeting signal may be a naturally occurring, recombinant, or mutant sequence positioned anywhere in a polypeptide or protein. An example for a naturally occurring mitochondrial targeting signal is the N-terminal region comprising amino acid residues 1 to 87 of the human OPA1 protein (NCBI Reference Sequence: NP_056375.2 from 11 Jul. 2020). Other mitochondrial import sequences are known in the art (see, for example, WO2006117250A2; U.S. Pat. Nos. 9,540,421B2; 6,316,652B1; US20110245146A1; U.S. Pat. No. 9,932,377; each of which is incorporated herein in its entirety by reference). The present invention discloses synthetic mitochondrial import sequences.

As used herein, the term “operatively linked” or “operatively combined” or “operably linked” or “operatively joined” or the like, when used to describe synthetic polypeptides or chimeric proteins, refer to polypeptide sequences that are placed in a physical and functional relationship to each other. In a most preferred embodiment, the functions of the polypeptide components of the chimeric molecule are unchanged compared to the functional activities of the parts in isolation. For example, a synthetic mitochondrial import signal of the present invention can be fused to a polypeptide of interest. In this case, it is preferable that the fusion molecule retains the function of the mitochondrial import signal and translocates the fusion protein to the mitochondria, and the polypeptide of interest retains its original biological activity. In some embodiments of the present invention, the activities of either the mitochondrial import signal or the protein of interest can be reduced relative to their activities in isolation. Such fusions can also find use with the present invention.

As used herein, the term “polypeptide” or “protein” refers to a polymer of four or more amino acid residues. The terms apply to amino acid polymers in which one or more amino acid residue is an artificial chemical analogue of a corresponding naturally occurring amino acid, as well as to naturally occurring amino acid polymers. The term “recombinant protein” refers to a protein that is produced by expression of a nucleotide sequence encoding the amino acid sequence of the protein from a recombinant DNA molecule.

As used herein, unless otherwise specified, the terms “peptide” and “polypeptide” refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

As used herein, the terms “wild-type”, “naturally occurring” and the like are used to refer to a protein, nucleic acid molecule, cell, or other material that occurs in nature. For example, a polypeptide or polynucleotide sequence that is present in an organism including in a virus. A naturally occurring material can be in its form as it exists in nature, and can be modified by the hand of man such that, for example, it is in an isolated form.

As used herein, the terms “synthetic”, “artificial”, “non-naturally occurring” and the like are used to refer to a polypeptide, protein, nucleic acid molecule, cell, or other material that does not occur in nature. For example, the mitochondrial import signals and fusion proteins provided by the present invention are non-naturally occurring because they consist of fragments of the OPA1 protein, or variants thereof, which are not found separate from the remainder of the naturally-occurring protein in nature.

As used herein, the term “identical,” when used in reference to two or more polynucleotide sequences or two or more polypeptide sequences, refers to the residues in the sequences that are the same when aligned for maximum correspondence. When percentage of sequence identity is used-in reference to a polypeptide, it is recognized that one or more residue positions that are not otherwise identical can differ by a conservative amino acid substitution, in which a first amino acid residue is substituted for another amino acid residue having similar chemical properties such as a similar charge or hydrophobic or hydrophilic character and, therefore, does not change the functional properties of the polypeptide. Where polypeptide sequences differ in conservative substitutions, the percent sequence identity can be adjusted upwards to correct for the conservative nature of the substitution. Such an adjustment can be made using well known methods, for example, scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions can be calculated using any-well known algorithm (see, for example, Meyers and Miller, Comp. Appl. Biol. Sci. (1988) 4:11-17; Smith and Waterman, Adv. Appl. Math. (1981) 2:482; Needleman and Wunsch, J. Mol. Biol. (1970) 48:443; Pearson and Lipman, Proc. Natl. Acad. Sci., (1988) 85:2444; Higgins and Sharp, Gene (1988) 73:237-244; Higgins and Sharp, CABIOS (1989) 5:151-153; Corpet et al., Nucl. Acids Res. (1988) 16:10881-10890; Huang, et al., Comp. Appl. Biol. Sci. (1992) 8:155-165, 1992; Pearson et al., (1994) 24:307-331). Alignment also can be performed by simple visual inspection and manual alignment of sequences.

As used herein, the term “sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “conservatively modified variation,” when used in reference to a particular polynucleotide sequence, refers to different polynucleotide sequences that encode identical or essentially identical amino acid sequences, or where the polynucleotide does not encode an amino acid sequence, to essentially identical sequences. Because of the degeneracy of the genetic code, a large number of functionally identical polynucleotides encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleotide sequence variations are “silent variations,” which can be considered a species of “conservatively modified variations.” As such, it will be recognized that each polynucleotide sequence disclosed herein as encoding a reporter protein variant also describes every possible silent variation. It will also be recognized that each codon in a polynucleotide, except AUG, which is ordinarily the only codon for methionine, and UUG, which is ordinarily the only codon for tryptophan, can be modified to yield a functionally identical molecule by standard techniques. Accordingly, each silent variation of a polynucleotide that does not change the sequence of the encoded polypeptide is implicitly described herein. Furthermore, it will be recognized that individual substitutions, deletions or additions that alter, add or delete a single amino acid or a small percentage of amino acids (typically less than 5%, and generally less than 1%) in an encoded sequence can be considered conservatively modified variations, provided alteration results in the substitution of an amino acid with a chemically similar amino acid.

Conservative amino acid substitutions providing functionally similar amino acids are well known in the art. Dependent on the functionality of the particular amino acid, i.e., catalytically important, structurally important, sterically important, different groupings of amino acid may be considered conservative substitutions for each other. The following list provides groupings of amino acids that are considered conservative substitutions based on the charge and polarity of the amino acid: 1) H, R and K; 2) D and E; 3) C, T, S, G, N, Q and Y; 4) A, P, M, L, I, V, F and W. The following list provides groupings of amino acids that are considered conservative substitutions based on the hydrophobicity of the amino acid: 1) D, E, N, K, Q and R; 2) C, S, I, P, G, H and Y; 3) A, M, I, L, V, F and W. The following list provides groupings of amino acids that are considered conservative substitutions based on the surface exposure/structural nature of the amino acid: 1) D, E, N, K, H, Q and R; 2) C, S, T, P, A, G, W and Y; 3) M, I, L, V and F. The following list provides groupings of amino acids that are considered conservative substitutions based on the secondary structure propensity of the amino acid: 1) A, E, Q, H, K, M, L and R; 2) C, T, I, V, F, Y and W; 3) S, G, P, D and N. The following list provides groupings of amino acids that are considered conservative substitutions based on their evolutionary conservation: 1) D and E; 2) H, K and R; 3) N and Q; 4) S and T; 5) L, and V; 6) F, Y and W; 7) A and G; 8) M and C.

Two or more amino acid sequences or two or more nucleotide sequences are considered to be “substantially identical” or “substantially similar” if the amino acid sequences or the nucleotide sequences share at least 80% sequence identity with each other, or with a reference sequence over a given comparison window. Thus, substantially similar sequences include those having, for example, at least 85% sequence identity, at least 90% sequence identity, at least 95% sequence identity, or at least 99% sequence identity. In certain embodiments, substantially similar sequences will have at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity.

A subject nucleotide sequence is considered “substantially complementary” to a reference nucleotide sequence if the complement of the subject nucleotide sequence is substantially identical to the reference nucleotide sequence. The term “stringent conditions” refers to a temperature and ionic conditions used in a nucleic acid hybridization reaction. Stringent conditions are sequence dependent and are different under different environmental parameters. Generally, stringent conditions are selected to be about 5° C. to 20° C. lower than the thermal melting point (Tm) for the specific sequence at a defined ionic strength and pH. The Tm is the temperature, under defined ionic strength and pH, at which 50% of the target sequence hybridizes to a perfectly matched probe.

As used herein, the term “variants” refers to polymorphic forms of a gene at a particular genetic locus, as well as cDNAs derived from mRNA transcripts of the genes, and the polypeptides encoded by them. The term “preferred mammalian codon” refers to the subset of codons from among the set of codons encoding an amino acid that are most frequently used in proteins expressed in mammalian cells as chosen from the following list: Gly (GGC, GGG); Glu (GAG); Asp (GAC); Val (GUG, GUC); Ala (GCC, GCU); Ser (AGC, UCC); Lys (AAG); Asn (AAC); Met (AUG); Ile (AUC); Thr (ACC); Trp (UGG); Cys (UGC); Tyr (UAU, UAC); Leu (CUG); Phe (UUC); Arg (CGC, AGG, AGA); Gln (CAG); H is (CAC); and Pro (CCC).

As used herein, the term “substantially” means that the recited characteristic, parameter, and/or value need not be achieved exactly, but that deviations or variations, including for example, tolerances, measurement error, measurement accuracy limitations and other factors known to skill in the art, may occur in amounts that do not preclude the effect the characteristic was intended to provide. A characteristic or feature that is substantially absent (e.g., substantially non-luminescent) may be one that is within the noise, beneath background, below the detection capabilities of the assay being used, or a small fraction (e.g., <1%, <0.1%, <0.01%, <0.001%, <0.00001%, <0.000001%, <0.0000001%) of the significant characteristic (e.g., luminescent intensity of a bioluminescent protein or bioluminescent complex).

As used herein, the term “complementary” or “complemental” refers to the characteristic of two or more structural elements (e.g., peptide, polypeptide, nucleic acid, small molecule, etc.) of being able to hybridize, dimerize, or otherwise form a complex with each other. For example, a “complementary peptide and polypeptide” are capable of coming together to form a complex. Complementary elements may require assistance to form a complex (e.g., from interaction elements), for example, by placing the elements in the proper conformation for complementarity, by co-localizing complementary elements, by lowering interaction energy for complementary, etc. Complementary elements may spontaneously form a complex when the elements are within in the proper proximity to one another.

As used herein, the term “complex” refers to an assemblage or aggregate of molecules (e.g., peptides, polypeptides, small molecules, etc.) in direct and/or indirect contact with one another. In one aspect, “contact,” or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules is formed under assay conditions such that the complex is thermodynamically favored (e.g., compared to a non-aggregated, or non-complexed, state of its component molecules). As used herein the term “complex,” unless described as otherwise, refers to the assemblage of two or more molecules (e.g., peptides, polypeptides, small molecules, or any combination thereof).

As used herein, the term “bioluminescence” refers to production and emission of light by a chemical reaction catalyzed by, or enabled by, an enzyme, protein, protein complex, or other biomolecule (e.g., bioluminescent complex). Examples of such enzymes (bioluminescent enzymes) include Oplophorus luciferase, firefly luciferase, click beetle luciferase, Renilla luciferase, cypridina luciferase, Aequorin photoprotein, obelin photoprotein and the like. In typical embodiments, a substrate for a bioluminescent enzyme is converted and emits light in the form of bioluminescence.

The term “luminescent enzyme,” “bioluminescent enzyme,” or “luciferase” as used interchangeably herein refers to a class of oxidative enzymes used in bioluminescence wherein the enzyme produces and emits light when given a substrate. The luciferase may be a naturally occurring, recombinant, or mutant luciferase that uses a luciferase substrate. The luciferase substrate may be luciferin, a luciferin derivative or analog, a preluciferin derivative or analog, a coelenterazine, or a coelenterazine derivative or analog. The luminescent enzyme, if naturally occurring, may be obtained easily by the skilled person from an organism. A person skilled in the art is readily able to further adapt, improve, or alter the properties of a bioluminescent enzyme to further enhance bioluminescence or other properties in context of present invention (see for example U.S. Pat. No. 10,202,584). If the luminescent enzyme is one that occurs naturally or is a recombinant or mutant luminescent enzyme, e.g. one which retains activity in a luciferase-coelenterazine or luciferase-luciferin reaction of a naturally occurring luminescent enzyme, it can be obtained readily from a culture of bacteria, yeast, mammalian cells, insect cells, plant cells, or the like, transformed to express a nucleic acid encoding the luminescent enzyme. Further, the recombinant or mutant luminescent enzyme can be derived from an in vitro cell-free system using a nucleic acid, and variants, recombinants, and mutants thereof.

As used herein, the term “non-luminescent” refers to an entity (e.g., peptide, polypeptide, complex, protein, etc.) that exhibits the characteristic of not emitting a detectable amount of light in the visible spectrum (e.g., in the presence of a substrate). For example, an entity may be referred to as non-luminescent if it does not exhibit detectable luminescence in a given assay. As used herein, the term “non-luminescent” is synonymous with the term “substantially non-luminescent.” For example, a non-luminescent polypeptide (NLpoly) is substantially non-luminescent, exhibiting, for example, a 10-fold or more (e.g., 100-fold, 200-fold, 500-fold, 1×103-fold, 1×104-fold, 1×105-fold, 1×106-fold, 1×107-fold, etc.) reduction in luminescence compared to a complex of the NLpoly with its non-luminescent complement peptide. In some embodiments, an entity is “non-luminescent” if any light emission is sufficiently minimal so as not to create interfering background for a particular assay.

As used herein, the terms “non-luminescent peptide” and “non-luminescent polypeptide” refer to peptides and polypeptides that exhibit substantially no luminescence (e.g., in the presence of a substrate), or an amount that is beneath the noise, or a 10-fold or more (e.g., 100-fold, 200-fold, 500-fold, 1×103-fold, 1×104-fold, 1×105-fold, 1×106-fold, 1×107-fold, etc.) when compared to a significant signal (e.g., luminescent complex) under standard conditions (e.g., physiological conditions, assay conditions, etc.) and with typical instrumentation (e.g., luminometer, etc.). In some embodiments, such non-luminescent peptides and polypeptides assemble, according to the criteria described herein, to form a bioluminescent complex. As used herein, a “non-luminescent element” is a non-luminescent peptide or non-luminescent polypeptide. The term “bioluminescent complex” refers to the assembled complex of two or more non-luminescent peptides and/or non-luminescent polypeptides. The bioluminescent complex catalyzes or enables the conversion of a substrate for the bioluminescent complex into an unstable form; the substrate subsequently emits light. When uncomplexed, two non-luminescent elements that form a bioluminescent complex may be referred to as a “non-luminescent pair.” If a bioluminescent complex is formed by three or more non-luminescent peptides and/or non-luminescent polypeptides, the uncomplexed constituents of the bioluminescent complex may be referred to as a “non-luminescent group.”

As used herein, the term “fluorescent protein” refers to any protein that can fluoresce when excited with an appropriate electromagnetic radiation, except that chemically tagged proteins, wherein the fluorescence is due to the chemical tag, are not considered fluorescent proteins for purposes of the present invention. In general, a fluorescent protein useful for use in a method of the invention is a protein that derives its fluorescence from autocatalytically forming a chromophore. A fluorescent protein can contain amino acid sequences that are naturally occurring or that have been engineered (i.e., variants or mutants). When used in reference to a fluorescent protein, the term “mutant” or “variant” refers to a protein that is different from a reference protein. For example, a spectral variant of Aequorea GFP can be derived from the naturally occurring GFP by engineering mutations such as amino acid substitutions into the reference GFP protein. Another nonlimiting example of a fluorescent protein according to the present invention is that derived from the Japanese eel UnaG and its variants (US 2016/0009771), each of which are incorporated herein by reference. Yet another example of a fluorescent protein according to the present invention is the cyan-excitable orange-red fluorescent protein (CyOFP) and its variants, which was derived from mNeptun2 by mutagenesis (U.S. Pat. No. 9,908,918), and which is incorporated herein with all its variants by reference.

By “derivative” or “derived from” is intended any suitable modification of the native polypeptide of interest, of a fragment of the native polypeptide, or of their respective analogs, such as glycosylation, phosphorylation, polymer conjugation (such as with polyethylene glycol), or other addition of foreign moieties, as long as the desired biological activity or fluorescence or bioluminescence characteristics of the native polypeptide is retained. Methods for making polypeptide fragments, analogs, and derivatives are generally available in the art.

The terms “bioluminescent fusion protein” and “bioluminescent fusion polypeptide,” as used herein, refer to a fusion protein comprising at least one fluorescent protein connected to at least one luciferase, wherein the fluorescent protein is operably linked to the luciferase to allow bioluminescence resonance energy transfer (BRET) between the fluorescent protein, which serves as a fluorescent BRET acceptor and a luciferase reaction product, which serves as a bioluminescent BRET donor upon reaction of a chemiluminescent substrate at the active site of the luciferase.

As used herein, the term “linker” refers to a moiety that assists in bringing together a pair of non-luminescent elements or a non-luminescent group to form a bioluminescent complex. In a typical embodiment, a linker is attached to a pair of non-luminescent elements (e.g., non-luminescent peptide/polypeptide pair), and the attractive interaction between the two interaction elements facilitates formation of the bioluminescent complex; although the present invention is not limited to such a mechanism, and an understanding of the mechanism is not required to practice the invention. Linker may facilitate formation of the bioluminescent complex by any suitable mechanism (e.g., bringing non-luminescent pair/group into close proximity, placing a non-luminescent pair/group in proper conformation for stable interaction, reducing activation energy for complex formation, combinations thereof, etc.). A linker may be a protein, polypeptide, peptide, small molecule, cofactor, nucleic acid, lipid, carbohydrate, antibody, etc. A linker may have additional functional properties, such as the binding of other proteins, enzymes and the like. In some embodiments, the linker may be recognized by the OMA1 protein. Such a linker may be referred to as a “recognition peptide”. The recognition peptide may further be hydrolyzed or its function in any other way abolished or altered in such a way that the operational complex of the elements connected by the linker alters its mode of operation. For example, in one embodiment the recognition peptide connects a fluorescent BRET acceptor and a bioluminescent BRET donor, and the OMA1 protease hydrolysis the recognition peptide and thereby alters the fluorescence spectrum of the BRET complex.

As used herein, the term “preexisting protein” refers to an amino acid sequence that was in physical existence prior to a certain event or date. A “peptide that is not a fragment of a preexisting protein” is a short amino acid chain that is not a fragment or sub-sequence of a protein (e.g., synthetic or naturally-occurring) that was in physical existence prior to the design and/or synthesis of the peptide.

As used herein and unless defined otherwise, the term “fragment” refers to a peptide or polypeptide that results from dissection or “fragmentation” of a larger whole entity (e.g., protein, polypeptide, enzyme, etc.), or a peptide or polypeptide prepared to have the same sequence as such. Therefore, a fragment is a subsequence of the whole entity (e.g., protein, polypeptide, enzyme, etc.) from which it is made and/or designed. A peptide or polypeptide that is not a subsequence of a preexisting whole protein is not a fragment (e.g., not a fragment of a preexisting protein). A peptide or polypeptide that is “not a fragment of a preexisting bioluminescent protein” is an amino acid chain that is not a subsequence of a protein (e.g., natural or synthetic) that: (1) was in physical existence prior to design and/or synthesis of the peptide or polypeptide, and (2) exhibits substantial bioluminescent activity. The fragment can include a C-terminal deletion an N-terminal deletion, and/or an internal deletion of the polypeptide. Active fragments of a particular protein or polypeptide will generally include at least about 5-10 contiguous amino acid residues of the full length molecule, preferably at least about 15-25 contiguous amino acid residues of the full length molecule, and most preferably at least about 20-50 or more contiguous amino acid residues of the full length molecule, or any integer between 4 amino acids and the full length sequence, provided that the fragment in question retains biological activity, such as catalytic activity, ligand binding activity, regulatory activity, or fluorescence, or bioluminescence characteristics, as defined herein.

As used herein, the term “subsequence” refers to peptide or polypeptide that has 100% sequence identify with another, larger peptide or polypeptide. The subsequence is a perfect sequence match for a portion of the larger amino acid chain.

As used herein, the “physiological conditions” encompasses any conditions compatible with living cells, e.g., predominantly aqueous conditions of a temperature, pH, salinity, chemical makeup, etc. that are compatible with living cells.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Sample may also refer to cell lysates or purified forms of the peptides and/or polypeptides described herein. Cell lysates may include cells that have been lysed with a lysing agent or lysates such as rabbit reticulocyte or wheat germ lysates. Sample may also include cell-free expression systems. Environmental samples include environmental material such as surface matter, soil, water, crystals and industrial samples. Such examples are not however to be construed as limiting the sample types applicable to the present invention.

### ASPECTS OF THE INVENTION

The present invention is based inter alia on the discovery that portions of OPA1's are still imported into mitochondria without the mitochondrial import sequence. OPA1's naturally occurring mitochondrial import sequence encompasses amino acids 1 to 87 (see NCBI Reference Sequence: NP_056375.2 from 11 Jul. 2020), so it was unexpected and surprising to see that fragments of OPA import sequence or even fragments upstream from OMA1's import sequence were capable of delivering a polypeptide into mitochondria. This discovery is against all teachings in the art.
(1) The inventor has contrived novel mitochondrial targeting sequences that stabilize a protein.
(2) The inventor has then engineered novel genes encoding reporter proteins that operatively combine such a synthetic targeting signal with an enzymatic function or reporter peptide.
(3) To this end, the inventor has further engineered novel synthetic enzymes and reporter peptides that operatively combine at least two elements with a recognition peptide in such a manner that the reporter or the enzymatic function is abolished upon cleavage of the recognition peptide.
(4) The inventor has contrived recognition peptides that are recognized by the OMA1 protease.
(5) The inventor has invented novel processes and methods to measure OMA1 protease activity with these new reporters. In order to further an understanding of the invention, a more detailed discussion is provided below regarding the reporter gene compositions of present invention and methods of their use.

In one aspect, the invention relates to novel compositions. Herein are provided novel reporter-genes that upon expression in a suitable host enable the in vivo measurement of OMA1 protease activity. These synthetic genes are built in a modular fashion and operatively combine 4 different functional elements: (a) a targeting signal; (b) an entity or fragment “N” of an enzymatic moiety or protein domain; (c) an entity or fragment “C” of an enzymatic moiety or protein domain that is corresponding to N; and (d) a sequence-motif that can be recognized by the OMA1 protease, whereby the complementation of N and C can produce a signal that can be measured. The targeting sequence and the recognition motive may—in certain embodiments—be one single entity as well. The term fragment as used above is merely illustrative to emphasize the functional complementation of these two elements or entities and is in no way limiting whatsoever to fragments of the same entity. In certain embodiments of present invention, the reporter-genes operatively combine two discrete polypeptides or enzymes or proteins to achieve desired effect; in other embodiments of present invention, the reporter-genes operatively combine two fragments of an enzyme or a polypeptide or a protein.

Non-limiting examples of reporter genes and their use are described in the Examples. It is to be understood that in some embodiments the N-terminal fragment and the C-terminal fragment may be arranged in the same order found in naturally-occurring enzymes or polypeptides or proteins. In some embodiments the N-terminal fragment and the C-terminal fragment may be arranged in a reversed (e.g., permutated) order found in naturally-occurring enzymes or polypeptides or proteins. In yet other embodiments, the operatively combined sequences are non-naturally occurring.

In certain embodiments, the reporter gene operatively combines a mitochondrial targeting peptide with a bioluminescent enzyme, which may consist of two or more non-luminescent peptides and/or non-luminescent polypeptides that are operatively combined is such a way that they become bioluminescent. In some embodiments, the non-luminescent peptides are fragments of a bioluminescent enzyme. In some embodiments, the bioluminescent enzyme is the NanoLuc luciferase (e.g., WO2014/151736). In certain embodiments, the reporter gene further comprises a recognition peptide, which operatively combines the non-luminescent peptides and/or non-luminescent polypeptides, and which may be a recognition peptide, which may be hydrolyzed by the OMA1 protease.

In certain embodiments, the reporter gene operatively combines a mitochondrial targeting peptide with two or more non-luminescent peptides and/or non-luminescent polypeptides and a recognition peptide, wherein the non-luminescent peptides and/or non-luminescent polypeptides assemble to form a bioluminescent enzyme upon hydrolysis of the recognition peptide. In some embodiments, the non-luminescent peptides are fragments of a luciferase.

In certain embodiments, the reporter gene operatively combines a mitochondrial targeting peptide with a fluorescent protein, which may consist of two or more non-fluorescent peptides and/or non-fluorescent polypeptides that are operatively combined is such a way that they become fluorescent. In some embodiments, the non-luminescent peptides are fragments of a fluorescent protein. In some embodiments the fluorescent protein is the UnaG protein. In certain embodiments, the reporter gene further comprises a recognition peptide, which operatively combines the non-fluorescent peptides and/or non-fluorescent polypeptides, and which may be a recognition peptide, which may be hydrolyzed by the OMA1 protease.

In certain embodiments, the reporter gene operatively combines a mitochondrial targeting peptide with a bioluminescent fusion protein, which may comprise at least one fluorescent protein connected to at least one luciferase, wherein the fluorescent protein is operably linked to the luciferase to allow bioluminescence resonance energy transfer (BRET) between the fluorescent protein, which serves as a fluorescent BRET acceptor and a luciferase reaction product, which serves as a bioluminescent BRET donor upon reaction of a chemilumninescent substrate at the active site of the luciferase. In certain embodiments, the reporter gene further comprises a recognition peptide, which operatively combines the fluorescent BRET acceptor and the bioluminescent BRET donor. In certain embodiments, the recognition peptide may be hydrolyzed by the OMA1 protease. An exemplary bioluminescent fusion protein comprises a NanoLuc luciferase linked to at least one CyOFP. In the presence of a chemiluminescent substrate, such as coelenterazine or a coelenterazine analog, such as furimazine, the bioluminescent fusion protein emits bright orange light as a result of bioluminescence resonance energy transfer from a luciferase reaction product to the CyOFP fluorophore(s).

Generally, a mitochondrial targeting peptide consists of 30 or more amino acids, preferably 80 amino acids, but not more than 160 amino acids. In one embodiment, the mitochondrial targeting peptide consists of from 50 to 150 amino acids. In another embodiment, the mitochondrial targeting peptide consists of from 50 to 140 amino acids. In another embodiment, the mitochondrial targeting peptide consists of from 50 to 130 amino acids. In another embodiment, the mitochondrial targeting peptide consists of from 50 to 120 amino acids. In another embodiment, the mitochondrial targeting peptide consists of from 50 to 110 amino acids. In another embodiment, the mitochondrial targeting peptide consists of from 50 to 100 amino acids. In another embodiment, the mitochondrial targeting peptide consists of from 50 to 90 amino acids. In another embodiment, the mitochondrial targeting peptide consists of from 60 to 100 amino acids. In another embodiment, the mitochondrial targeting peptide consists of from 70 to 100 amino acids. In another embodiment, the mitochondrial targeting peptide consists of from 80 to 100 amino acids. In another embodiment, the mitochondrial targeting peptide consists of from 80 to 90 amino acids. In another embodiment, the mitochondrial targeting peptide consists of 86 amino acids. In yet other embodiments, the mitochondrial targeting peptide consists of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 amino acids.

Generally, a recognition peptide consists of 4 or more amino acids, preferably 23 amino acids, but not more than 50 amino acids. In one embodiment, the recognition peptide consists of from 10 to 50 amino acids. In another embodiment, the recognition peptide consists of from 10 to 40 amino acids. In another embodiment, the recognition peptide consists of from 10 to 30 amino acids. In another embodiment, the recognition peptide consists of from 10 to 20 amino acids. In another embodiment, the recognition peptide consists of 23 amino acids. In yet other embodiments, the recognition peptide consists of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids.

In certain embodiments, the mitochondrial targeting peptide of the reporter gene may comprise the amino acid sequence of SEQ ID NO: 17 or SEQ ID NO: 19 or SEQ ID NO: 21 or SEQ ID NO: 23 or SEQ ID NO: 25 or SEQ ID NO: 27 or variations or combinations thereof.

In certain embodiments, the recognition peptide of the reporter gene may comprise the amino acid sequence of SEQ ID NO: 33 or SEQ ID NO: 35 or SEQ ID NO: 37 or SEQ ID NO: 39 or SEQ ID NO: 41 or SEQ ID NO: 43 or SEQ ID NO: 45 or SEQ ID NO: 47 or variations or combinations thereof.

In certain embodiments, the fragment “N” of the reporter gene may comprise the amino acid sequence of SEQ ID NO: 49 or SEQ ID NO: 51 or SEQ ID NO: 53 or SEQ ID NO: 55 or SEQ ID NO: 57 or SEQ ID NO: 59 or variations or combinations thereof.

In certain embodiments, the fragment “C” of the reporter gene may comprise the amino acid sequence of SEQ ID NO: 61 or SEQ ID NO: 63 or SEQ ID NO: 65 or SEQ ID NO: 67 or SEQ ID NO: 69 or SEQ ID NO: 71 or SEQ ID NO: 73 or variations or combinations thereof.

In certain embodiments, the reporter gene may comprise the amino acid sequence of SEQ ID NO: 49 and SEQ ID NO: 61.

In certain embodiments, the reporter gene may comprise the amino acid sequence of SEQ ID NO: 51 and SEQ ID NO: 63.

In certain embodiments, the reporter gene may comprise the amino acid sequence of SEQ ID NO: 51 and SEQ ID NO: 65.

In certain embodiments, the reporter gene may comprise the amino acid sequence of SEQ ID NO: 53 and SEQ ID NO: 67.

In certain embodiments, the reporter gene may comprise the amino acid sequence of SEQ ID NO: 55 and SEQ ID NO: 69.

In certain embodiments, the reporter gene may comprise the amino acid sequence of SEQ ID NO: 57 and SEQ ID NO: 71.

In certain embodiments, the reporter gene may comprise the amino acid sequence of SEQ ID NO: 59 and SEQ ID NO: 73.

In certain embodiments, the reporter gene may comprise the amino acid sequence of SEQ ID NO: 17 and SEQ ID NO: 33.

In certain embodiments, the reporter gene may comprise the amino acid sequence of SEQ ID NO: 19 and SEQ ID NO:35.

In certain embodiments, the reporter gene may comprise the amino acid sequence of SEQ ID NO: 19 and SEQ ID NO:37.

In certain embodiments, the reporter gene may comprise the amino acid sequence of SEQ ID NO: 21 and SEQ ID NO:39.

In certain embodiments, the reporter gene may comprise the amino acid sequence of SEQ ID NO: 23 and SEQ ID NO:41.

In certain embodiments, the reporter gene may comprise the amino acid sequence of SEQ ID NO: 25 and SEQ ID NO:43.

In certain embodiments, the reporter gene may comprise the amino acid sequence of SEQ ID NO: 27 and SEQ ID NO: 45.

In certain embodiments, the reporter gene may comprise the amino acid sequence of SEQ ID NO: 27 and SEQ ID NO: 33.

In certain embodiments, the reporter gene may comprise the amino acid sequence of SEQ ID NO: 33, SEQ ID NO: 49 and SEQ ID NO: 61.

In certain embodiments, the reporter gene may comprise the amino acid sequence of SEQ ID NO:35, SEQ ID NO: 51 and SEQ ID NO: 63.

In certain embodiments, the reporter gene may comprise the amino acid sequence of SEQ ID NO:35, SEQ ID NO: 51 and SEQ ID NO: 65.

In certain embodiments, the reporter gene may comprise the amino acid sequence of SEQ ID NO:37, SEQ ID NO: 53 and SEQ ID NO: 67.

In certain embodiments, the reporter gene may comprise the amino acid sequence of SEQ ID NO:39, SEQ ID NO: 55 and SEQ ID NO: 69.

In certain embodiments, the reporter gene may comprise the amino acid sequence of SEQ ID NO:41, SEQ ID NO: 57 and SEQ ID NO: 71.

In certain embodiments, the reporter gene may comprise the amino acid sequence of SEQ ID NO:43, SEQ ID NO: 59 and SEQ ID NO: 73.

In certain embodiments, the reporter gene may comprise the amino acid sequence of SEQ ID NO: 45, SEQ ID NO: 49 and SEQ ID NO: 61.

In certain embodiments, the reporter gene may comprise the amino acid sequence of SEQ ID NO: 33, SEQ ID NO: 49 and SEQ ID NO: 61.

In certain embodiments, the reporter gene may comprise the amino acid sequence of SEQ ID NO: 17, SEQ ID NO: 49 and SEQ ID NO: 61.

In certain embodiments, the reporter gene may comprise the amino acid sequence of SEQ ID NO: 19, SEQ ID NO: 51 and SEQ ID NO: 63.

In certain embodiments, the reporter gene may comprise the amino acid sequence of SEQ ID NO: 19, SEQ ID NO: 51 and SEQ ID NO: 65.

In certain embodiments, the reporter gene may comprise the amino acid sequence of SEQ ID NO: 19, SEQ ID NO: 53 and SEQ ID NO: 67.

In certain embodiments, the reporter gene may comprise the amino acid sequence of SEQ ID NO: 21, SEQ ID NO: 55 and SEQ ID NO: 69.

In certain embodiments, the reporter gene may comprise the amino acid sequence of SEQ ID NO: 23, SEQ ID NO: 57 and SEQ ID NO: 71.

In certain embodiments, the reporter gene may comprise the amino acid sequence of SEQ ID NO: 25, SEQ ID NO: 59 and SEQ ID NO: 73.

In certain embodiments, the reporter gene may comprise the amino acid sequence of SEQ ID NO: 27, SEQ ID NO: 49 and SEQ ID NO: 61.

In certain embodiments, the reporter gene may comprise the amino acid sequence of SEQ ID NO: 17, SEQ ID NO: 33, SEQ ID NO: 49 and SEQ ID NO: 61.

In certain embodiments, the reporter gene may comprise the amino acid sequence of SEQ ID NO: 19, SEQ ID NO:35, SEQ ID NO: 51 and SEQ ID NO: 63.

In certain embodiments, the reporter gene may comprise the amino acid sequence of SEQ ID NO: 19, SEQ ID NO:35, SEQ ID NO: 51 and SEQ ID NO: 65.

In certain embodiments, the reporter gene may comprise the amino acid sequence of SEQ ID NO: 19, SEQ ID NO:37, SEQ ID NO: 53 and SEQ ID NO: 67.

In certain embodiments, the reporter gene may comprise the amino acid sequence of SEQ ID NO: 21, SEQ ID NO:39, SEQ ID NO: 55 and SEQ ID NO: 69.

In certain embodiments, the reporter gene may comprise the amino acid sequence of SEQ ID NO: 23, SEQ ID NO:41, SEQ ID NO: 57 and SEQ ID NO: 71.

In certain embodiments, the reporter gene may comprise the amino acid sequence of SEQ ID NO: 25, SEQ ID NO:43, SEQ ID NO: 59 and SEQ ID NO: 73.

In certain embodiments, the reporter gene may comprise the amino acid sequence of SEQ ID NO: 27, SEQ ID NO: 45, SEQ ID NO: 49 and SEQ ID NO: 61.

In certain embodiments, the reporter gene may comprise the amino acid sequence of SEQ ID NO: 27, SEQ ID NO: 33, SEQ ID NO: 49 and SEQ ID NO: 61.

In one embodiment, the reporter gene comprises the amino acid sequence of SEQ ID NO: 01 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto.

In one embodiment, the reporter gene comprises the amino acid sequence of SEQ ID NO: 03 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto.

In one embodiment, the reporter gene comprises the amino acid sequence of SEQ ID NO: 05 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto.

In one embodiment, the reporter gene comprises the amino acid sequence of SEQ ID NO: 07 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto.

In one embodiment, the reporter gene comprises the amino acid sequence of SEQ ID NO: 09 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto.

In one embodiment, the reporter gene comprises the amino acid sequence of SEQ ID NO: 11 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto.

In one embodiment, the reporter gene comprises the amino acid sequence of SEQ IL) NO: 13 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto.

In one embodiment, the reporter gene comprises the amino acid sequence of SEQ ID NO: 15 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto.

In one embodiment, the reporter gene comprises the amino acid sequence of SEQ IL) NO: 01 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, wherein the mitochondrial targeting sequence does not comprise the amino acid sequence of SEQ ID NO: 29.

In another particular embodiment, the reporter gene comprises the amino acid sequence of SEQ ID NO: 15 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, wherein the mitochondrial targeting sequence does not comprise the amino acid sequence of SEQ ID NO: 31.

In some embodiments, polypeptides or proteins are provided comprising the amino acid sequence of SEQ ID NO: 01, SEQ ID NO: 03, SEQ ID NO: 05, SEQ ID NO: 07, SEQ ID NO: 09, SEQ ID NO: 11, SEQ ID NO: 13, or SEQ ID NO: 15 with one or snore additions, substitutions, and/or deletions.

In some embodiments, a peptide or polypeptide and/or protein of present invention comprises a synthetic peptide, peptide containing one or more non-natural amino acids, peptide mimetic, conjugated synthetic peptide (e.g., conjugated to a functional group (e.g., fluorophore, luminescent substrate, etc.)).

It is understood that a reporter gene of present invention may comprise one or more linkers operatively combining the sequences. Linkers are typically short peptide sequences of 2-30 amino acid residues, often composed of glycine and/or serine residues. Linker amino acid sequences will typically be short, e.g., 20 or fewer amino acids (i.e., 20, 19, 18, 17, 16, 15, 14, 13, 12, 11, 10, 9, 8, 7, 6, 5, 4, 3, 2, or 1). Examples include short peptide sequences which facilitate cloning, poly-glycine linkers (Glyn where n=2, 3, 4, 5, 6, 7, 8, 9, 10 or more), histidine tags (Hisn where n=3, 4, 5, 6, 7, 8, 9, 10 or more), linkers composed of glycine and serine residues ([Gly-Ser]n, [Gly-Gly-Ser-Gly]n, [Ser-Ala-Gly-Gly]n, and [Gly-Gly-Gly-Gly-Ser]n, wherein n=1, 2, 3, 4, 5, 6, 7, 8, 9, 10, 11, 12, 13, 14, 15 or more), GSAT, SEG, and Z-EGFR linkers. Linkers may include restriction sites, which aid cloning and manipulation. Other suitable linker amino acid sequences will be apparent to those skilled in the art. (See e.g., Argos P. J. Biol. (1990) 211(4):943-958; Crasto et al. Protein Eng. (2000) 13:309-312; George et al. Protein Eng. (2002) 15:871-879; Arai et al. Protein Eng. (2001). 14:529-532; and the Registry of Standard Biological Parts (partsregistry.org/Protein_domains/Linker).

In certain embodiments, tag sequences may be added to the reporter genes of present invention. In some embodiments, tag sequences are located at the N-terminus or C-terminus of the reporter gene. In other embodiments, tag sequences may be inserted at any position within the reporter gene. Exemplary tags that can be used in the practice of the invention include a His-tag, a Strep-tag, a TAP-tag, an S-tag, an SBP-tag, an Arg-tag, a calmodulin-binding peptide tag, a cellulose-binding domain tag, a DsbA-tag, a c-myc tag, a glutathione S-transferase tag, a FLAG-tag, a HAT-tag, a maltose-binding protein tag, a NusA-tag, and a thioredoxin tag.

Reporter genes may also be fused with additional fluorescent or bioluminescent proteins, or biologically active domains or polypeptide fragments, or variants thereof having fluorescence or bioluminescence characteristics (e.g., green fluorescent protein (GFP) or luciferase).

Any luciferase may be used to construct a reporter gene. Luciferase sequences from a number of species are well known in the art, such as, but not limited to, deep-sea shrimp Oplophorus luciferase, firefly luciferase, click beetle luciferase, Renilla luciferase, Gaussia luciferase, Metridia luciferase, Vargula luciferase, bacterial luciferase (e.g., Vibrio fischeri, haweyi, and harveyi), and dinoflagellate luciferase, any of which can be incorporated into a bioluminescent fusion protein. Representative luciferase sequences are shown in the National Center for Biotechnology Information (NCBI) database. See, for example, NCBI entries: Accession Nos. JQ437370, AFJ15586, AHH41349, AHH41346, HV216898, HV216897, Q9GV45, AB644228, M63501, AY015988, EF535511, AY015993, EU239244, AB371097, AB371096, EU025117, AB519703, AB674506, U89490, M25666, XM_003190150, XM_003602031, YP_004273613, YP_004216833, YP_003275551, KEP44836, YP_004213749, EFR93032, YP_206879, YP_206878, ABG26273, WP_005438583, WP_005384122, P07740, EF492542, AF085332, AF394060, AF394059, EU025117; AY364164, U03687, M65067; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a bioluminescent fusion protein, or a nucleic acid encoding a bioluminescent fusion protein, as described herein.

In certain embodiments, the reporter gene comprises a luciferase derived from Oplophonis gracihrostris. Such bioluminescent fusion proteins can produce light from chemiluminescent substrates, including coelenterazine and coelenterazine analogs (see, e.g., U.S. Patent Application Publication No. 20120117667; herein incorporated by reference in its entirety).

In one embodiment, the bioluminescent fusion protein comprises NanoLuc luciferase, an engineered Oplophorus gracilirostris luciferase variant available from Promega Corporation (Madison, Wis.). NanoLuc luciferase is a 19.1 kDa, ATP-independent luciferase that utilizes the coelenterazine analog, furimazine, as a chemiluminescent substrate to produce high intensity luminescence. A representative amino acid sequence of NanoLuc luciferase is presented in SEQ ID NO: 51. In one embodiment, a polypeptide comprising the sequence of SEQ ID NO: 51 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, is used to construct a reporter gene encoding a protein, wherein the luciferase is capable of catalyzing a light-producing reaction with a chemiluminescent substrate that can be used for measuring OMA1 protease activity.

Any fluorescent protein may be used to construct a reporter gene. Examples for fluorescent proteins include, but are not limited to, green fluorescent proteins (GFPs), cyan fluorescent proteins (CFPs), blue fluorescent proteins (BFPs) and yellow fluorescent proteins (YFPs), where the color of the fluorescence depends on the wavelength of the emitted light; green fluorescent proteins emit light in the range of 520-565 nm; cyan fluorescent proteins emit light in the range of 500-520 nm; blue fluorescent proteins emit light in the range of 450-500 nm; yellow fluorescent proteins emit light in the range of 565-590 nm; and red fluorescent proteins, described further below, emit light in the range of 625-740 nm. Furthermore, fluorescent proteins useful in the invention include, for example, those which have been genetically engineered for improved properties such as, without limitation, improved protein expression; altered excitation or emission wavelengths; enhanced brightness, pH resistance, stability or speed of fluorophore formation or fluorophore disassembly; photoactivation; or reduced oligomerization or photobleaching.

Fluorescent proteins useful in the invention encompass those which emit in a variety of spectra, including violet, blue, cyan, green, yellow, orange and red. As described further below, fluorescent proteins useful in the invention also include, yet are not limited to, blue fluorescent proteins (BFPs) and cyan fluorescent proteins (CFPs) produced by random mutagenesis of GFP and rationally designed yellow fluorescent proteins (YFPs). BFP has a Tyr66His substitution relative to GFP that shifts the absorbance spectrum to a peak of 384 nm with emission at 448 nm (Heim et al., Proc. Natl. Acad. Sci. (1994) 91:12501). CFP, which is brighter and more photostable than UP, has an absorption/emission spectral range intermediate between BFP and EGFP due to a Tyr66Trp substitution (Heim et al., Proc. Natl. Acad. Sci. (1994) 91:12501; Heim and Tsien, Curr. Biol. (1996) 6:178-182; and Ellenberg et al., Biotechniques (1998) 25:838); the Thr203Tyr CFP variant known as “CGFP” has excitation and emission wavelengths intermediate between CFP and EGFP. The rationally designed YFP has red-shifted absorbance and emission spectra with respect to green fluorescent proteins (Ormo et al., Science 273:1392 (1996); Heim and Tsien, supra, 1996). A variety of YFP variants display improved characteristics including, without limitation, the YFP variants “Citrine” (YFP-Val68Leu/Gln69Met; Griesbeck et al., J. Biol. Chem. (2001) 276:29188-29194) and “Venus” (YFP-Phe46Leu/Phe64Leu/Met153Thr/Val 63Ala/Ser175Gly), an extremely bright and fast-maturing YFP (Nagai et al., Nature Biotech. (2002) 20:87-90). One skilled in the art understands that these and a variety of other fluorescent proteins which are derived, for example, from GFP or other naturally occurring fluorescent proteins, also can be useful in the invention.

A fluorescent protein useful in the invention also can be a long wavelength fluorescent protein such as a red or far-red fluorescent protein, which can be useful for reducing or eliminating background fluorescence from samples derived from eukaryotic cells or tissues. Such red fluorescent proteins include naturally occurring and genetically modified forms of Discosoma striata proteins including, without limitation, DsRed (DsRed1 or drFP583; Matz et al., Nat. Biotech. (1999) 17:969-973); dsRed2 (Terskikh et al., J. Biol. Chem. (2002) 277:7633-7636); T1 (dsRed-Express; Clontech; Palo Alto, Calif.; Bevis and Glick, Nature Biotech. (2002) 20:83-87); and the dsRed variant mRFP1 (Campbell et al., Proc. Natl. Acad. Sci. USA (2002) 99:7877-7882). Such red fluorescent proteins further include naturally occurring and genetically modified forms of Heteractis crispa proteins such as HcRed (Gurskaya et al., FEBS Lett. (2001) 507:16).

Fluorescent proteins useful in a reporter gene can be derived from any of a variety of species including marine species such as A. victoria and other coelenterate marine organisms. Useful fluorescent proteins encompass, without limitation, Renilla mulled-derived fluorescent proteins such as the dimeric Renilla mulleri GFP, which has narrow excitation (498 nm) and emission (509 nm) peaks (Peele et al., J. Prot. Chem. (2001) 507-519); Anemonia sulcata fluorescent proteins such as DsRed proteins, for example, asFP595 (Lukyanov et al., J. Biol. Chem. (2000) 275: 25879-25882); Discosoma fluorescent proteins, for example, Discosoma striata red fluorescent proteins such as dsFP593 (Fradkov et al., FEBS Lett. (2000) 479:127-130); Heteractis crispa fluorescent proteins such as HcRed and HcRed-2A (Gurskaya et al., FEBS Lett. (2001) 507:16); and Entacmeae quadricolor fluorescent proteins including red fluorescent proteins such as eqFP611 (Wiedenmann et al., Proc. Natl. Acad. Sci. USA (2002) 99:11646-11651), all of which sequences are herein incorporated by reference. One skilled in the art understands that these and many other fluorescent proteins, including species homologs of the above described naturally occurring fluorescent proteins as well as engineered fluorescent proteins can be useful in designing reporter genes of the invention.

In certain embodiments, the fluorescent protein according to the present invention is a polypeptide having fluorescent properties in the presence of bilirubin. This fluorescent protein is a group of polypeptides possessing a common characteristic of emitting fluorescence having a prescribed wavelength by irradiation with excitation light in the presence of bilirubin or analogous, but not emitting fluorescence by irradiation with the same excitation light in the absence of bilirubin or analogous. An example of the fluorescent polypeptide with such properties is that derived from eel, more specifically, derived from Japanese eel, and known as UnaG (SEQ ID NO: 75) and its variants, all of which sequences are herein incorporated by reference. Although UnaG was originally isolated from Japanese eel, the origin of the fluorescent polypeptide is not limited thereto.

In one particular embodiment, the reporter gene comprises the sequence or subsequences of the amino acid sequence of SEQ ID NO: 75 or a variant thereof comprising a sequence having at least about sequence identity thereto, including any percent identity within this range, such as 81, 82, 83, 84, 85, 86, 87, 88, 89, 90, 91, 92, 93, 94, 95, 96, 97, 98, or 99% sequence identity thereto, wherein the subsequences are operatively combined to a fluorescent protein. This fluorescent protein may be any polypeptide composed of amino acids joined by peptide bonds, but is not limited thereto. For example, the fluorescent polypeptide may contain a structure other than polypeptides. Non-limited examples of the structure other than the polypeptide include carbohydrate chains and isoprenoid groups.

The invention also provides bioluminescent fusion proteins comprising at least one fluorescent protein and at least one luciferase, wherein the fluorescent protein is operably linked to the luciferase to allow bioluminescence resonance energy transfer (BRET) between the fluorescent protein, which serves as a fluorescent BRET acceptor, and a luciferase reaction product, which serves as a bioluminescent BRET donor.

In certain embodiments, the bioluminescent fusion protein comprises a fluorescent protein comprising the amino acid sequence of SEQ ID NO: 63 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, wherein the fluorescent protein emits orange-red light in response to absorption of cyan excitation light.

In some embodiments, the invention includes a bioluminescence resonance energy transfer (BRET) system comprising a bioluminescent fusion protein, as described herein, and a chemiluminescent substrate (e.g., coelenterazine, coelenterazine analog (e.g., furimazine), or other luciferase substrate). The BRET system may further comprise a photodetector or imaging device for detecting light emitted from the bioluminescent fusion protein, such as, but not limited to, an optical microscope, a digital microscope, a luminometer, a charged coupled device (CCD) image sensor, a complementary metal-oxide-semiconductor (CMOS) image sensor, or a digital camera.

In certain embodiments, the polynucleotide sequence encoding a reporter gene polypeptide is codon optimized for expression in a bacterial host cell, e.g., E. coli. In other embodiments, the polynucleotide sequence encoding a reporter gene polypeptide is codon optimized for expression in a eukaryotic host cell or organism, e.g., a fungi, yeast, worm, mouse, rat, hamster, guinea pig, monkey, or human. In yet other embodiments, the polynucleotide sequence encoding a reporter gene polypeptide is codon optimized for expression in a mammalian host cell or organism, e.g., a mouse, rat, hamster, guinea pig, monkey. In some embodiments, polynucleotide sequences encoding a reporter gene polypeptide are provided comprising the sequence of SEQ ID NO: 02, SEQ ID NO: 04, SEQ ID NO: 06, SEQ ID NO: 08, SEQ ID NO: 10, SEQ ID NO: 12, SEQ ID NO: 14, or SEQ ID NO: 16 or a variant thereof

In certain embodiments, the invention provides an expression vector, comprising expression control sequences operatively linked to a nucleic acid molecule encoding a reporter gene polypeptide. In other embodiments, the invention provides a virus comprising a nucleic acid molecule encoding a reporter gene polypeptide. In yet other embodiments, the invention provides a recombinant host cell comprising a nucleic acid molecule encoding a reporter gene polypeptide.

Another aspect of the invention relates to methods comprising the herein disclosed reporter. With the provided disclosures and examples a person having ordinary skills in the art can easily understand the metes and bounds of the invention, make modifications, variations or design similar methods, reporter and/or assays, which are all within the scope of present invention. Reporter are widely used to study many aspects of various fields, such as biology, chemistry, or medicine. As such, according to the disclosures provided herein, the present invention pertains also to methods that find use in various fields. Applications may include but are not limited to clinical disease monitoring, diagnostics, therapeutic drug monitoring, biological research, pharmaceuticals, compound detection and monitoring, etc. Additional applications include drug development, such as high throughput screening of molecules or safety and toxicology studies.

Methods of use according to the present invention comprise a reporter, which may comprise a targeting signal, complemental elements, and a recognition domain, wherein the recognition domain separates the complemental elements in such a way that the elements are functional. The recognition particle may form a complex, according to present invention, whereby the recognition particle is hydrolyzed and the functional elements separated. This typically results in abolishment of the element's function, which can be measured and correlated to a complex formation or another relevant event.

In certain embodiments, the reporter genes are expressed in a host, such as a cell or a whole organism. The recombinant host is then exposed to at least one experimental condition and a signal of certain strength is generated depending on the condition. Alternatively, the reporter gene may be isolated from a host and used in an assay. Methods of protein isolation are known to those of skill in the art and are described in, e.g., Protein Purification Applications: A Practical Approach, (Simon Roe, Ed., 2001). A signal may be detected in an assay by any suitable means, which may be direct or indirect and which may comprise inter alia a photodetector or imaging device, such as, but not limited to, an optical microscope, a digital microscope, a luminometer, a charged coupled device (CCD) image sensor, a complementary metal-oxide-semiconductor (CMOS) image sensor, a photomultiplier tube, or a digital camera. The signal may further be processed, integrated or compared to signals obtained under other conditions (e.g., control conditions), and correlated to the experimental conditions.

In some embodiments, such a signal is detected by measuring a change in a detectable label (i.e. a detectable moiety) that is part of the reporter. In some embodiments, the reporter molecule contains detectable moieties which provide for an indication of a cleavage event. In some embodiments, cleavage may be detected by size changes in the length of the polypeptide (e.g., gel electrophoresis, size exclusion column chromatography, immunoflourescence, etc.) or other biochemical and physical changes that occur to the reporter molecule. In some embodiments, the reporter molecule comprises a label which facilitates cleavage detection. In some embodiments, the reporter molecule comprises a cleavable enzyme (e.g., a bioluminescent enzyme), wherein a cleavage event alters the enzyme's function. In some embodiments, the reporter molecule comprises a cleavable detectable moiety (e.g., a fluorescent protein), wherein a cleavage event alters the moiety and this alteration is detected. In some embodiments, cleavage is detected using a FRET-based pair or a BRET-based pair, wherein a change in fluorescence is indicative of a cleavage event. Methods for detecting and monitoring cleavage of proteins are well known and any such methods may be employed in detecting cleavage of the reporter molecules of present invention. In some embodiments reporter of present invention are combined with at least one other reporter for dual readout, or in certain embodiments, multimodal readout.

In certain embodiments, the signals from such assays may be correlated to the formation of a complex, which may comprise any number of a small molecule, a compound, a molecule, a peptide, a polypeptide or a protein or other compositions. In certain embodiments, the signal is correlated to a complex formed by a compound with a polypeptide or a protein (e.g. a drug-target interaction). In certain embodiments, the signal is correlated to a complex formed by a molecule with a protease, such as the OMA1 protease. Such a method is useful inter alia for the identification of protease inhibitors, such as OMA1 inhibitors. In some embodiments, methods of screening for OMA1 inhibitors are provided, wherein OMA1 hydrolyzes the recognition peptide thereby abolishing the signal of the reporter. The screen thus identifies potential OMA1 inhibitors as compounds or molecules that are capable of preserving the reporter's signal, which is useful for reducing the likelihood of identifying potential false-hits (e.g., compounds that obstruct the signal generated by the functional elements rather than inhibiting OMA1).

In some embodiments, the invention provides a method of detecting the presence of one or more protease activities in a sample comprising a) combining the sample with a reporter molecule comprising a targeting signal, complemental elements of a detectable moiety, and a recognition element, wherein the recognition element separates the complemental elements in such a way that the detectable moiety is functional; and b) detecting cleavage of the recognition element by the separation of the complemental elements and a change of the detectable moiety's function. In some embodiments, one protease activity can be detected with such a method. In some embodiments, 2, 3, 4, 5, 6, 7, 8, 9 or 10 protease activities can be detected with such a method. In some embodiments, one or more protease activities can be detected with such a method. In some embodiments, OMA1 protease activity can be detected with such a method. In other embodiments, YME1L1 protease activity can be detected with such a method. In yet other embodiments, AFG3L2 protease activity can be detected with such a method. In certain embodiments, i-AAA protease activity can be detected with such a method. In other embodiments, m-AAA protease activity can be detected with such a method. In yet other embodiments, PARL protease activity can be detected with such a method.

The invention provides a method of identifying protease inhibitors, said method comprising the steps of: a) combining a molecule with a reporter protein comprising a complemental elements of a detectable moiety and a recognition element; and b) detecting a change of the detectable moiety's function; wherein the molecule is recognized as protease inhibitor when the detectable moiety's function is not significantly altered. Optionally the method may further comprise the step of allowing for protease activity to occur after step a) (e.g., a step of activating the protease). Optionally the reporter protein may further comprise a targeting signal.

The invention provides a method of identifying cell-permeable protease inhibitors, said method comprising the steps of: a) combining a molecule with a recombinant host expressing a reporter protein comprising a complemental elements of a detectable moiety and a recognition element, wherein the recognition element separates the complemental elements in such a way that the detectable moiety is functional; and b) detecting cleavage of the recognition element by the separation of the complemental elements and a change of the detectable moiety's function; wherein the molecule is recognized as protease inhibitor when the detectable moiety's function is not significantly altered. Optionally the method may further comprise a step of allowing for protease activity to occur after step a) (e.g., a step of activating the protease). Optionally the reporter protein may further comprise a targeting signal.

The invention provides a method of screening for cell-permeable protease inhibitors, said method comprising the steps of a) obtaining a compound library; b) exposing a recombinant host expressing a reporter protein comprising a complemental elements of a detectable moiety and a recognition element, to a compound of said library; and c) measuring a change of the detectable moiety's function; wherein the compound is recognized as protease inhibitor when the detectable moiety's function is not significantly altered. Optionally the method may further comprise the step of allowing for protease activity to occur after step a) (e.g., a step of activating the protease). Optionally the reporter protein may further comprise a targeting signal.

In other embodiments, methods of measuring a compound's mitochondrial toxicity are provided, wherein the recognition peptide becomes hydrolyzed upon exposure to a compound and the reporter signal abolished. These methods are particularly useful for determining toxic concentrations in cellular assays by correlating a compound's concentration and the strength of the signal from the reporter (e.g., a dose-response relationship). In some embodiments, present invention provides methods of testing for toxicity (or toxicity potential) which may be used to select compounds which are, or are predicted to be, or have the potential to be, suitable for in vivo administration without adverse toxicity, such as without adverse mitochondrial toxicity.

The invention provides a method of predicting in vivo toxicity (such as mitochondrial toxicity) of a molecule, said method comprising the steps of: a) combining the molecule with a recombinant host expressing a reporter protein comprising a complemental elements of a detectable moiety and a recognition element, wherein the recognition element separates the complemental elements in such a way that the detectable moiety is functional; and b) detecting cleavage of the recognition element by the separation of the complemental elements and a change of the detectable moiety's function; wherein the detection of increased cleavage indicates increased toxicity of the molecule. Optionally the reporter protein may further comprise a targeting signal. Optionally the method may further comprise the step of allowing for protease activity to occur after step a) (e.g., a step of activating the protease).

The invention provides a method of selecting a compound with reduced in vivo toxicity (such as mitochondrial toxicity), said method comprising the steps of: a) combining the compound with a recombinant host expressing a reporter protein comprising a complemental elements of a detectable moiety, and a recognition element, wherein the recognition element separates the complemental elements in such a way that the detectable moiety is functional; b) detecting cleavage of the recognition element by the separation of the complemental elements and a change of the detectable moiety's function; and c) selecting one or more compounds for which no cleavage or reduced cleavage was detected. Optionally the method may further comprise the step d) administering the selected compound to a mammal, such as a human subject, after step c). Optionally the reporter protein may further comprise a targeting signal. Optionally the method may further comprise the step of allowing for protease activity to occur after step a) (e.g., a step of activating the protease).

Provided herein are several formats for use of the reporter genes in assays. In some embodiments, these are performed in vitro and in other embodiments, they are performed in vivo, in yet other embodiments ex vivo. In some embodiments, the reporter genes are transiently expressed in a host cell and in other embodiments stably transfected cells express the reporter. Recombinant genes, recombinant protein and recombinant cells or recombinant organisms can be supplied individually, combined or as a kit, as separate diagnostic and/or research kit components, or as stand-alone reagents customizable to the individual assay.

Such kits can comprise reporter genes together with suitable instructions and other necessary reagents for preparing or using them as described above. The kit may contain in separate containers a reporter gene polypeptide or recombinant constructs for producing a reporter gene polypeptide, and/or cells (either already transfected or separate). Additionally, instructions (e.g., written, tape, VCR, CD-ROM, DVD, flash drive, SD card, etc.) for using reporter of present invention, for example, as a reporter for determining mitochondrial toxicity may be included in the kit. The kit may also contain other packaged reagents and materials (e.g., transfection reagents, buffers, media, and the like). As discussed above, the reporter can be used inter alia in fluorescent or bioluminescent assays. Therefore, kits may also include reagents for performing such assays or medical imaging. In certain embodiments, the kit further includes a chemiluminescent substrate, a BRET system, or a reporter gene construct utilizing a fluorescent protein or bioluminescent fusion protein, as described herein.

Reporter genes of present invention can be produced in any number of ways, all of which are well known in the art. Those skilled in the art can further introduce a mutation by an arbitrary method in order to enhance at least one property of the polypeptides, enzymes and/or proteins encoded by the reporter genes of present invention, such as signal-to-noise ratio, signal stability, signal specificity and signal strength.

In one embodiment, the provided reporter genes are generated using recombinant techniques. One of skill in the art can also readily determine nucleotide sequences that encode the desired polypeptides using standard methodology and the teachings herein. Basic texts disclosing the general methods of recombinant techniques include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)). Recombinant techniques are readily used to clone sequences encoding polypeptides useful in the claimed invention that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci USA (1982) 79:6409. The sequences encoding polypeptides can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge Nature (1981) 292:756; Nambair et al. Science (1984) 223:1299; Jay et al. J. Biol. Chem. (1984) 259:6311; Stemmer et al. Gene (1995) 164:49-53.

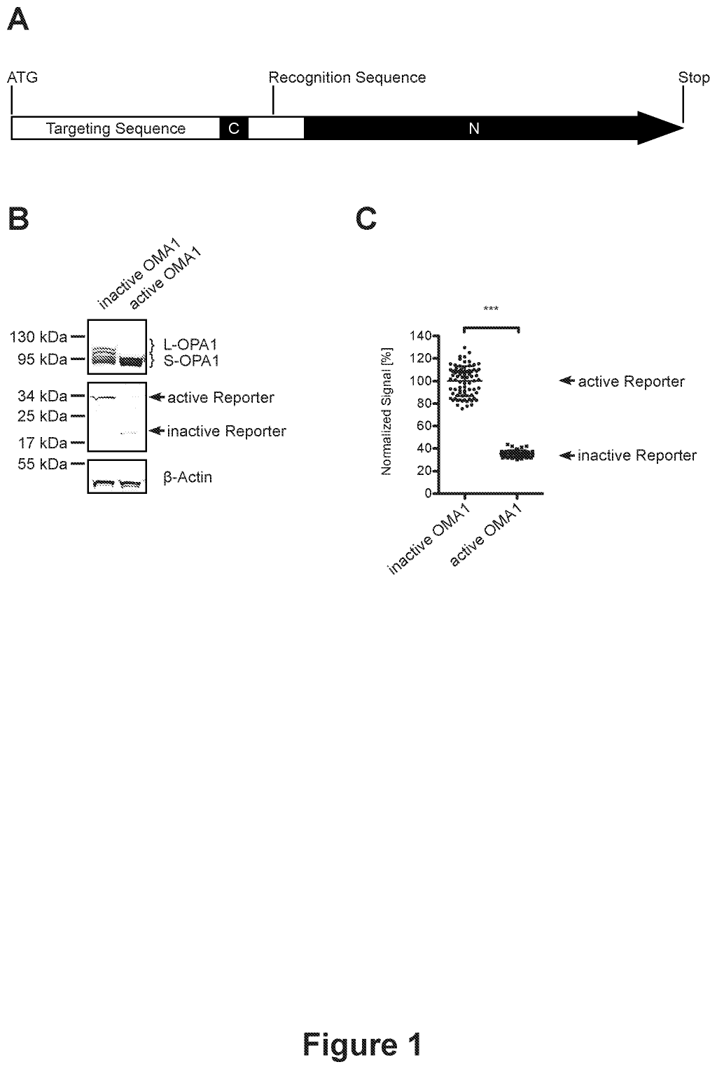

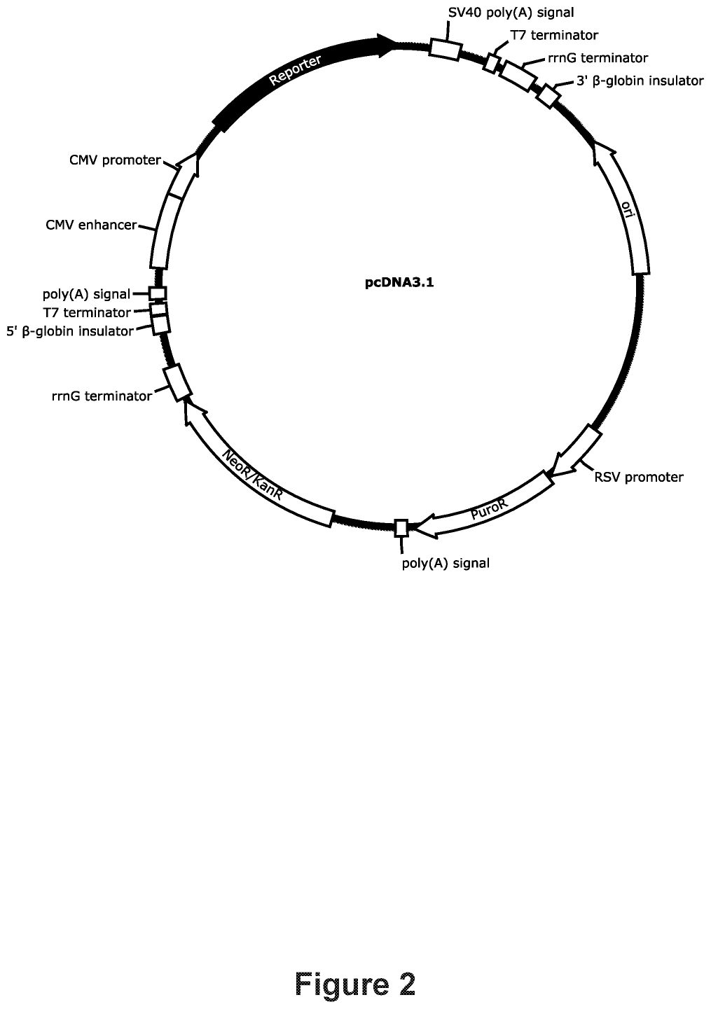

Once coding sequences have been isolated and/or synthesized, they can be cloned into any suitable vector or replicon for expression. As will be apparent from the teachings herein, a wide variety of vectors encoding polypeptides of present invention can be generated for expression in prokaryotic or eukaryotic cells. A nonlimiting example for such a vector is depicted in FIG. 1A. The skilled artisan readily able to select or design suitable vectors useful in the context of present invention. Numerous cloning vectors are known to those of skill in the art, and the selection of an appropriate cloning vector is a matter of choice.

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. For example, expression of proteins from eukaryotic vectors can be regulated using inducible promoters. With inducible promoters, expression levels are tied to the concentration of inducing agents, such as tetracycline or ecdysone, by the incorporation of response elements for these agents into the promoter. Generally, high-level expression is obtained from inducible promoters only in the presence of the inducing agent; basal expression levels are minimal. Inducible expression vectors are often chosen if expression of the protein of interest is detrimental to eukaryotic cells. Other types of regulatory elements may also be present in the vector.

Typically, the expression vector is used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Hek293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter glia, Aedes aegypti, Autographa californica, Bombyx mori, Drosophila melanogaster, Spodoptera frupperda, and Trichoplusia ni.

Depending on the expression system and host selected, the reporter genes of present invention are produced by growing host cells transformed by an expression vector described above under conditions whereby the protein encoded by the reporter gene of interest is expressed. The selection of the appropriate growth conditions is within the skill of the art.

In some embodiments, the reporter genes of present invention are placed within an expression cassette useful for expression in eukaryotic cells. Expression cassettes typically include control elements operably linked to the coding sequence, which allow for the expression of the gene in vivo in the subject species. For example, typical promoters for mammalian cell expression include the SV40 early promoter, a CMV promoter such as the CMV immediate early promoter, the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence.

Enhancer elements may also be used herein to increase expression levels of the mammalian constructs. Examples include the SV40 early gene enhancer, as described in Dijkema et al., EMPO J. (1985) 4:761, the enhancer/promoter derived from the long terminal repeat (LTR) of the Rous Sarcoma Virus, as described in Gorman et al., Proc. Natl. Acad. Sci. USA (1982) 79:6777 and elements derived from human CMV, as described in Boshart et al., Cell (1985) 41:521, such as elements included in the CMV intron A sequence.

Alternative targeting sequences may also be used to direct localization of the polypeptides or proteins encoded by the reporter to a specific tissue, cell-type (e.g. muscle, heart, or neural cell), cellular compartment (e.g., mitochondria or other organelle, plasma membrane), or protein. For example, constructs may include a polynucleotide sequence encoding a secretory protein signal sequence, a membrane protein signal sequence, a nuclear localization sequence, a nucleolar localization signal sequence, an endoplasmic reticulum localization sequence, a peroxisome localization sequence, a mitochondrial localization sequence, or a protein-protein interaction motif sequence, See, e.g., Protein Targeting, Transport, and Translocation (R. Dalbey and Gunnar von Heijne eds., Academic Press, 2002); Protein Targeting Protocols (Methods in Molecular Biology, R. A. Clegg ed., Humana Press, 1998); Protein Engineering and Design (S. J. Park J. R. Cochran eds., CRC Press, 2009); Protein-Protein Interactions: Methods and Applications (Methods in Molecular Biology, H. Fu ed., Humana Press, 2004); Emanuelsson et al. Biochim. Biophys. Acta (2001) 1541(1-2):114-119; Hurley et al. Annu. Rev. Biophys. Biomol. Struct. (2000) 29:49-79; Jans et al. Bioessays (2000) 22(6):532-544; Christophe et al. Cell Signal. (2000) 12(5):337-341; Stanley Mol. Membr. Biol. (1996) 13(1):19-27; Cosson et al. Cold Spring Harb. Symp. Quant. Biol. (1995) 60:113-117; Emmott et al. EMBO Rep. (2009) 10(3):231-238; Gurkan et al. Adv. Exp. Med. Biol. (2007) 607:73-83; Romanelli et al. J. Neurochem. (2008) 105(6):2055-2068; Terlecky et al. Adv. Drug Deliv. Rev. (2007) 59(8):739-747; Arnoys et al. Acta Histochem. (2007) 109(2):89-110; Brown et al. Kidney Int. (2000) 57(3):816-824; Jadwin et al. FEBS Lett. (2012) 586(17):2586-2596; Liu et al, FEBS Lett. (2012) 586(17):2597-2605; Romero et al. Adv. Pharmacol. (2011) 62:279-314; Obenauer et al. Methods Mol. Biol. (2004) 261:445-468; each of which herein incorporated by reference.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl2 method by procedures well known in the art. Alternatively, MgCl2 or RbCl can be used. Transformation can also be performed after forming a protoplast of the host cell or by electroporation.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be co-transfected with DNA sequences encoding the reporter molecules of the invention, and a second foreign DNA molecule encoding a selectable phenotype, such as the herpes simplex thymidine kinase gene.

A number of viral based systems have been developed for gene transfer into mammalian cells. These include inter alia adenoviruses, retroviruses (γ-retroviruses and lentiviruses), poxviruses, adeno-associated viruses, baculoviruses, and herpes simplex viruses (see c.a., Warnock et al. Methods Mol. Biol. (2011) 737:1-25; Walther et al. Drugs (2000) 60(2):249-271; and Lundstrom Trends Biotechnol. (2003) 21(3):117-122; herein incorporated by reference). In one embodiment of present invention, reporter gene #01 is delivered to High-Five insect cells via a baculovirus.

In certain embodiments, reporter genes may be used to generate recombinant host cells by integration (i.e., knock-in) of a transgene into the chromosome of a eukaryotic cell. Such a knock-in may be random or site-specific and preferentially involves a mammalian cell. Many methods for knocking in of a transgene into a host are known in the arts. A typical process for site-specific integration involves the steps of 1) introducing a targeting vector containing a gene of interest into eukaryotic cells and 2) screening and selecting transfected cells with integration of the gene of interest at specific genomic locus.

In some embodiments, cells are transiently transfected or stably transformed or transfected with one or multiple vectors coding for the reporter gene(s) (e.g., comprising a targeting signal, two complemental elements and a recognition element). In some embodiments, transgenic organisms are generated that code for the necessary reporter protein for carrying out the assays described herein. In other embodiments, the reporter genes are expressed in cells from a subject, such as lymphoblasts, skin fibroblasts or myoblasts.

Cell-free reconstituted systems may be used for the expression of the herein provided reporter genes as well. Typically, such systems may comprise cellular lysates derived for the simultaneous translation, or coupled transcription and translation, of recombinant genetic materials encoding experimental and control reporter enzymes or proteins.

Another aspect of the invention relates to methods of therapy of a pathological condition or a disease amenable to OMA1 modulators.

In certain embodiments, a drug with OMA1 modulating properties is Tipranavir, Pazopanib hydrochloride, Sorafenib, Sunitinib, Ibrutinib, Regorafenib, Celecoxib, Raloxifene, Dactinomycin, Enasidenib, Cabozantinib, Tamoxifen citrate, Pexidartinib, Daunorubicin hydrochloride, Dabrafenib mesylate, Lodatinib, Valrubicin, Trametinib, Entrectinib, Bosutinib, Idarubicin hydrochloride, Tucatinib, Selinexor, Ribociclib, Ceritinib, Imatinib, Doxorubicin hydrochloride, Venetoclax, Gilteritinib, Mitotane, or Osimertinib.

In one aspect, Pazopanib hydrochloride or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Pazopanib hydrochloride for use in treating a pathological condition or disease characterized by pathological OMA1 levels or OMA1 activity.

In one aspect, Pazopanib hydrochloride is used for the making of a personalized medicine for treating a pathological condition or disease characterized by pathological OMA1 levels or OMA1 activity.

In one aspect, a process uses Pazopanib hydrochloride as an ingredient for the preparation of a personalized medicine for treating a pathological condition or disease characterized by pathological OMA1 levels or OMA1 activity.

In one aspect, Sorafenib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Sorafenib for use in treating a pathological condition or disease characterized by pathological OMA1 levels or OMA1 activity.

In one aspect, Sorafenib is used for the making of a personalized medicine for treating a pathological condition or disease characterized by pathological OMA1 levels or OMA1 activity.

In one aspect, a process uses Sorafenib as an ingredient for the preparation of a personalized medicine for treating a pathological condition or disease characterized by pathological OMA1 levels or OMA1 activity.

In one aspect, Kavain or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Kavain for use in treating a pathological condition or disease characterized by pathological OMA1 levels or OMA1 activity.

In one aspect, Kavain is used for the making of a personalized medicine for treating a pathological condition or disease characterized by pathological OMA1 levels or OMA1 activity.

In one aspect, a process uses Kavain as an ingredient for the preparation of a personalized medicine for treating a pathological condition or disease characterized by pathological OMA1 levels or OMA1 activity.

In one aspect, Sunitinib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Sunitinib for use in treating a pathological condition or disease characterized by pathological OMA1 levels or OMA1 activity.

In one aspect, Sunitinib is used for the making of a personalized medicine for treating a pathological condition or disease characterized by pathological OMA1 levels or OMA1 activity.

In one aspect, a process uses Sunitinib as an ingredient for the preparation of a personalized medicine for treating a pathological condition or disease characterized by pathological OMA1 levels or OMA1 activity.

In one aspect, Ibrutinib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Ibrutinib for use in treating a pathological condition or disease characterized by pathological OMA1 levels or OMA1 activity.

In one aspect, Ibrutinib is used for the making of a personalized medicine for treating a pathological condition or disease characterized by pathological OMA1 levels or OMA1 activity.

In one aspect, a process uses Ibrutinib as an ingredient for the preparation of a personalized medicine for treating a pathological condition or disease characterized by pathological OMA1 levels or OMA1 activity.

In one aspect, Regorafenib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Regorafenib for use in treating a pathological condition or disease characterized by pathological OMA1 levels or OMA1 activity.

In one aspect, Regorafenib is used for the making of a personalized medicine for treating a pathological condition or disease characterized by pathological OMA1 levels or OMA1 activity.

In one aspect, a process uses Regorafenib as an ingredient for the preparation of a personalized medicine for treating a pathological condition or disease characterized by pathological OMA1 levels or OMA1 activity.

In one aspect, Celecoxib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Celecoxib for use in treating a pathological condition or disease characterized by pathological OMA1 levels or OMA1 activity.

In one aspect, Celecoxib is used for the making of a personalized medicine for treating a pathological condition or disease characterized by pathological OMA1 levels or OMA1 activity.

In one aspect, a process uses Celecoxib as an ingredient for the preparation of a personalized medicine for treating a pathological condition or disease characterized by pathological OMA1 levels or OMA1 activity.

In one aspect, Raloxifene or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Raloxifene for use in treating a pathological condition or disease characterized by pathological OMA1 levels or OMA1 activity.

In one aspect, Raloxifene is used for the making of a personalized medicine for treating a pathological condition or disease characterized by pathological OMA1 levels or OMA1 activity.

In one aspect, a process uses Raloxifene as an ingredient for the preparation of a personalized medicine for treating a pathological condition or disease characterized by pathological OMA1 levels or OMA1 activity.

In one aspect, Dactinomycin or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Dactinomycin for use in treating a pathological condition or disease characterized by pathological OMA 1 levels or OMA1 activity.

In one aspect, Dactinomycin is used for the making of a personalized medicine for treating a pathological condition or disease characterized by pathological OMA1 levels or OMA1 activity.

In one aspect, a process uses Dactinomycin as an ingredient for the preparation of a personalized medicine for treating a pathological condition or disease characterized by pathological OMA1 levels or OMA1 activity.

In one aspect, Enasidenib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Enasidenib for use in treating a pathological condition or disease characterized by pathological OMA1 levels or OMA1 activity.

In one aspect, Enasidenib is used for the making of a personalized medicine for treating a pathological condition or disease characterized by pathological OMA1 levels or OMA1 activity.

In one aspect, a process uses Enasidenib as an ingredient for the preparation of a personalized medicine for treating a pathological condition or disease characterized by pathological OMA1 levels or OMA1 activity.

In one aspect, Cabozantinib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Cabozantinib for use in treating a pathological condition or disease characterized by pathological OMA1 levels or OMA1 activity.

In one aspect, Cabozantinib is used for the making of a personalized medicine for treating a pathological condition or disease characterized by pathological OMA1 levels or OMA1 activity.

In one aspect, a process uses Cabozantinib as an ingredient for the preparation of a personalized medicine for treating a pathological condition or disease characterized by pathological OMA1 levels or OMA1 activity.

In one aspect, Tamoxifen citrate or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Tamoxifen citrate for use in treating a pathological condition or disease characterized by pathological OMA1 levels or OMA1 activity.

In one aspect, Tamoxifen citrate is used for the making of a personalized medicine for treating a pathological condition or disease characterized by pathological OMA1 levels or OMA1 activity.

In one aspect, a process uses Tamoxifen citrate as an ingredient for the preparation of a personalized medicine for treating a pathological condition or disease characterized by pathological OMA1 levels or OMA1 activity.

In one aspect, Pexidartinib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Pexidartinib for use in treating a pathological condition or disease characterized by pathological OMA1 levels or OMA1 activity.

In one aspect, Pexidartinib is used for the making of a personalized medicine for treating a pathological condition or disease characterized by pathological OMA1 levels or OMA1 activity.

In one aspect, a process uses Pexidartinib as an ingredient for the preparation of a personalized medicine for treating a pathological condition or disease characterized by pathological OMA1 levels or OMA1 activity.

In one aspect, Daunorubicin hydrochloride or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Daunorubicin hydrochloride for use in treating a pathological condition or disease characterized by pathological OMA1 levels or OMA1 activity.

In one aspect, Daunorubicin hydrochloride is used for the making of a personalized medicine for treating a pathological condition or disease characterized by pathological OMA1 levels or OMA1 activity.

In one aspect, a process uses Daunorubicin hydrochloride as an ingredient for the preparation of a personalized medicine for treating a pathological condition or disease characterized by pathological OMA1 levels or OMA1 activity.

In one aspect, Dabrafenib mesylate or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Dabrafenib mesylate for use in treating a pathological condition or disease characterized by pathological OMA1 levels or OMA1 activity.

In one aspect, Dabrafenib mesylate is used for the making of a personalized medicine for treating a pathological condition or disease characterized by pathological OMA1 levels or OMA1 activity.

In one aspect, a process uses Dabrafenib mesylate as an ingredient for the preparation of a personalized medicine for treating a pathological condition or disease characterized by pathological OMA1 levels or OMA1 activity.

In one aspect, Lorlatinib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Lorlatinib for use in treating a pathological condition or disease characterized by pathological OMA1 levels or OMA1 activity.

In one aspect, Lorlatinib is used for the making of a personalized medicine for treating a pathological condition or disease characterized by pathological OMA1 levels or OMA1 activity.

In one aspect, a process uses Lorlatinib as an ingredient for the preparation of a personalized medicine for treating a pathological condition or disease characterized by pathological OMA1 levels or OMA1 activity.

In one aspect, Valrubicin or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Valrubicin for use in treating a pathological condition or disease characterized by pathological OMA1 levels or OMA1 activity.

In one aspect, Valrubicin is used for the making of a personalized medicine for treating a pathological condition or disease characterized by pathological OMA1 levels or OMA1 activity.

In one aspect, a process uses Valrubicin as an ingredient for the preparation of a personalized medicine for treating a pathological condition or disease characterized by pathological OMA1 levels or OMA1 activity.

In one aspect, Trametinib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Trametinib for use in treating a pathological condition or disease characterized by pathological OMA1 levels or OMA1 activity.

In one aspect, Trametinib is used for the making of a personalized medicine for treating a pathological condition or disease characterized by pathological OMA1 levels or OMA1 activity.

In one aspect, a process uses Trametinib as an ingredient for the preparation of a personalized medicine for treating a pathological condition or disease characterized by pathological OMA1 levels or OMA1 activity.

In one aspect, Entrectinib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Entrectinib for use in treating a pathological condition or disease characterized by pathological OMA 1 levels or OMA1 activity.

In one aspect, Entrectinib is used for the making of a personalized medicine for treating a pathological condition or disease characterized by pathological OMA1 levels or OMA1 activity.

In one aspect, a process uses Entrectinib as an ingredient for the preparation of a personalized medicine for treating a pathological condition or disease characterized by pathological OMA1 levels or OMA1 activity.

In one aspect, Bosutinib or a solvate, prodrug, analogue, or pharmaceutically acceptable salt thereof is used in a method of therapy of a pathological condition or disease characterized by pathological OMA1 levels or OMA 1 activity.

In one aspect, a pharmaceutical composition comprises Bosutinib for use in treating a pathological condition or disease characterized by pathological OMA1 levels or OMA1 activity.

In one aspect, Bosutinib is used for the making of a personalized medicine for treating a pathological condition or disease characterized by pathological OMA1 levels or OMA1 activity.

In one aspect, a process uses Bosutinib as an ingredient for the preparation of a personalized medicine for treating a pathological condition or disease characterized by pathological OMA1 levels or OMA1 activity.

In one aspect, Idarubicin hydrochloride or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Idarubicin hydrochloride for use in treating a pathological condition or disease characterized by pathological OMA1 levels or OMA1 activity.

In one aspect, Idarubicin hydrochloride is used for the making of a personalized medicine for treating a pathological condition or disease characterized by pathological OMA1 levels or OMA1 activity.

In one aspect, a process uses Idarubicin hydrochloride as an ingredient for the preparation of a personalized medicine for treating a pathological condition or disease characterized by pathological OMA1 levels or OMA 1 activity.

In one aspect, Tucatinib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Tucatinib for use in treating a pathological condition or disease characterized by pathological OMA1 levels or OMA1 activity.

In one aspect, Tucatinib is used for the making of a personalized medicine for treating a pathological condition or disease characterized by pathological OMA1 levels or OMA1 activity.

In one aspect, a process uses Tucatinib as an ingredient for the preparation of a personalized medicine for treating a pathological condition or disease characterized by pathological OMA1 levels or OMA1 activity.

In one aspect, Selinexor or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Selinexor for use in treating a pathological condition or disease characterized by pathological OMA1 levels or OMA1 activity.

In one aspect, Selinexor is used for the making of a personalized medicine for treating a pathological condition or disease characterized by pathological OMA1 levels or OMA1 activity.

In one aspect, a process uses Selinexor as an ingredient for the preparation of a personalized medicine for treating a pathological condition or disease characterized by pathological OMA1 levels or OMA1 activity.

In one aspect, Ribociclib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Ribociclib for use in treating a pathological condition or disease characterized by pathological OMA1 levels or OMA1 activity.

In one aspect, Ribociclib is used for the making of a personalized medicine for treating a pathological condition or disease characterized by pathological OMA1 levels or OMA1 activity.

In one aspect, a process uses Ribociclib as an ingredient for the preparation of a personalized medicine for treating a pathological condition or disease characterized by pathological OMA1 levels or OMA1 activity.

In one aspect, Ceritinib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Ceritinib for use in treating a pathological condition or disease characterized by pathological OMA1 levels or OMA1 activity.

In one aspect, Ceritinib is used for the making of a personalized medicine for treating a pathological condition or disease characterized by pathological OMA1 levels or OMA1 activity.

In one aspect, a process uses Ceritinib as an ingredient for the preparation of a personalized medicine for treating a pathological condition or disease characterized by pathological OMA1 levels or OMA1 activity.

In one aspect, Imatinib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Imatinib for use in treating a pathological condition or disease characterized by pathological OMA1 levels or OMA1 activity.

In one aspect, Imatinib is used for the making of a personalized medicine for treating a pathological condition or disease characterized by pathological OMA1 levels or OMA1 activity.

In one aspect, a process uses Imatinib as an ingredient for the preparation of a personalized medicine for treating a pathological condition or disease characterized by pathological OMA1 levels or OMA1 activity.

In one aspect, Doxorubicin hydrochloride or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Doxorubicin hydrochloride for use in treating a pathological condition or disease characterized by pathological OMA1 levels or OMA1 activity.

In one aspect, Doxorubicin hydrochloride is used for the making of a personalized medicine for treating a pathological condition or disease characterized by pathological OMA1 levels or OMA1 activity.

In one aspect, a process uses Doxorubicin hydrochloride as an ingredient for the preparation of a personalized medicine for treating a pathological condition or disease characterized by pathological OMA1 levels or OMA1 activity.

In one aspect, Venetoclax or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Venetoclax for use in treating a pathological condition or disease characterized by pathological OMA1 levels or OMA1 activity.

In one aspect, Venetoclax is used for the making of a personalized medicine for treating a pathological condition or disease characterized by pathological OMA1 levels or OMA1 activity.

In one aspect, a process uses Venetoclax as an ingredient for the preparation of a personalized medicine for treating a pathological condition or disease characterized by pathological OMA1 levels or OMA1 activity.

In one aspect, Gilteritinib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Gilteritinib for use in treating a pathological condition or disease characterized by pathological OMA 1 levels or OMA1 activity.

In one aspect, Gilteritinib is used for the making of a personalized medicine for treating a pathological condition or disease characterized by pathological OMA1 levels or OMA1 activity.

In one aspect, a process uses Gilteritinib as an ingredient for the preparation of a personalized medicine for treating a pathological condition or disease characterized by pathological OMA1 levels or OMA1 activity.

In one aspect, Mitotane or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Mitotane for use in treating a pathological condition or disease characterized by pathological OMA1 levels or OMA1 activity.

In one aspect, Mitotane is used for the making of a personalized medicine for treating a pathological condition or disease characterized by pathological OMA1 levels or OMA1 activity.

In one aspect, a process uses Mitotane as an ingredient for the preparation of a personalized medicine for treating a pathological condition or disease characterized by pathological OMA1 levels or OMA1 activity.

In one aspect, Osimertinib or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Osimertinib for use in treating a pathological condition or disease characterized by pathological OMA1 levels or OMA1 activity.

In one aspect, Osimertinib is used for the making of a personalized medicine for treating a pathological condition or disease characterized by pathological OMA1 levels or OMA1 activity.

In one aspect, a process uses Osimertinib as an ingredient for the preparation of a personalized medicine for treating a pathological condition or disease characterized by pathological OMA1 levels or OMA1 activity.

In one aspect, Tipranavir or a solvate, prodrug, analogue, or pharmaceutically acceptable salt thereof is used in a method of therapy of a pathological condition or disease characterized by pathological OMA1 levels or OMA1 activity.

In one aspect, a pharmaceutical composition comprises Tipranavir for use in treating a pathological condition or disease characterized by pathological OMA1 levels or OMA1 activity.

In one aspect, Tipranavir is used for the making of a personalized medicine for treating a pathological condition or disease characterized by pathological OMA1 levels or OMA1 activity.

In one aspect, a process uses Tipranavir as an ingredient for the preparation of a personalized medicine for treating a pathological condition or disease characterized by pathological OMA1 levels or OMA1 activity.

It is understood that the aspects and embodiments provided herein are for illustrative purposes only and that various modifications or changes in light thereof will be suggested to persons skilled in the art and are to be included within the spirit and purview of this application and scope of the appended claims.

### EXAMPLES

Below are examples of specific embodiments for carrying out the present invention. The examples are offered for illustrative purposes only, and are not intended to limit the scope of the present invention in any way whatsoever. Efforts have been made to ensure accuracy with respect to numbers used (e.g., concentrations), but some experimental error and deviation should, of course, be allowed for.

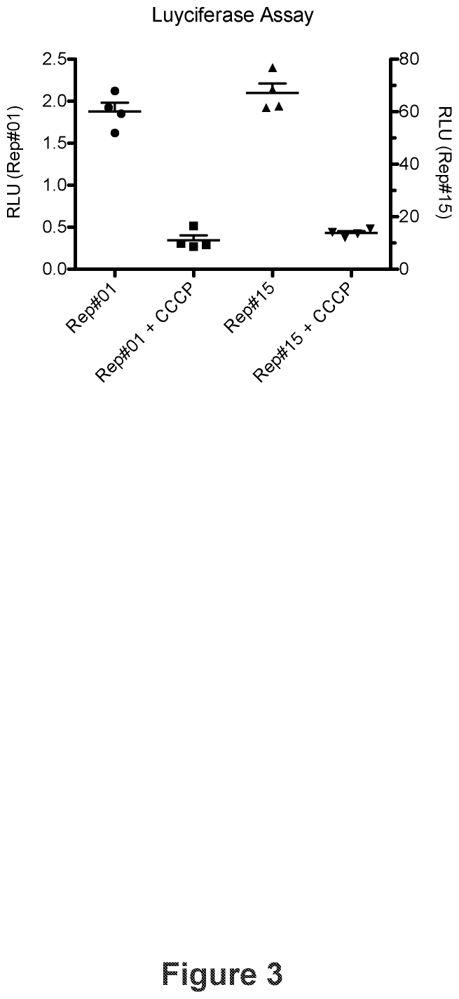

Example 1 provides a nonlimiting example of generation and use of a reporter of present invention and illustrates its function. To this end, generation and use of reporter gene #01 (SEQ ID NO: 01) and reporter gene #15 (SEQ ID NO: 15) are described and their function compared to each other. The general design of a reporter of present invention is illustrated in FIG. 1A and its working principle in FIG. 1B. A reporter in general, and reporter gene #01 and reporter gene #15 in particular, can be generated as follows. DNA sequences of a reporter may be codon-optimized in silico or by any other way for expression in a suitable host. DNA sequences of reporter genes #01 and #15 were codon-optimized for expression in humans (SEQ ID NO: 02 and SEQ ID NO: 16, respectively). The (optimized) sequences were synthesized and cloned into a pcDNA3.1 expression vector under the control of a CMV promotor (see FIG. 1 for an illustration of such vector). Of course, an expression system and a host should match for best results. The host in this example were Hek293T cells, which were maintained under standard culture conditions in DMEM with 10% Fetal Bovine Serum and 1% Pen/Strep. For an experiment, Hek293T cells were seeded in opaque white tissue-culture treated 96-well plates at 80% confluency and transfected with vectors carrying reporter gene #01 and reporter gene #15 and 24 hours later incubated for 30 minutes at 37° C. in OptiMEM or in OptiMEM with 10 μM CCCP. After 30 minutes, the OptiMEM cell-culture media was replaced with luciferase substrate (furimazine, Promega) diluted 1:100 in OptiMEM, and bioluminescence was measured with a Fluoroskan Ascent FL (Thermo Scientific) using a 517 nm single-pass filter and 200 milliseconds integration time. The results are provided as bar-graph with standard deviations in FIG. 3. This merely illustrative example demonstrates: (A) the bioluminescence signal from both reporter #01 and #15 was significantly reduced upon CCCP-treatment. CCCP-treatment leads to OMA1 activation and hydrolysis of the reporter peptides encoded by the reporter genes #01 and #15. This cleavage event disabled the reporter activity and thereby abolished the bioluminescence signal. And (B) reporter #15 produced a much stronger signal compared to reporter #01 by almost an order of magnitude. (Note the two different scales of the two y-axes in FIG. 3). This finding was very surprising and against all expectations. Reporter #01 comprises a large part of OPA1's amino-terminus as mitochondrial targeting sequence, which is cleaved by the mitochondrial processing peptidase upon mitochondrial import (see Example 2). Reporter #15 was initially envisioned as a control for reporter #01. Reporter #15 therefore lacks the first 66 amino acids of reporter #01 believed to be essential for mitochondrial import. Every person having ordinary skills in the art, and even a person having extra-ordinary skills in the art, such as myself, would have predicted that reporter #15 is retained in the cytoplams and not translocated into mitochondria, because of the missing mitochondrial import sequence. It is surprising and against all expectations to find reporter #15 translocate into mitochondria (see also Example 7). Yet, it is mind-boggling to see a much better performance of reporter #15 compared to reporter #01.

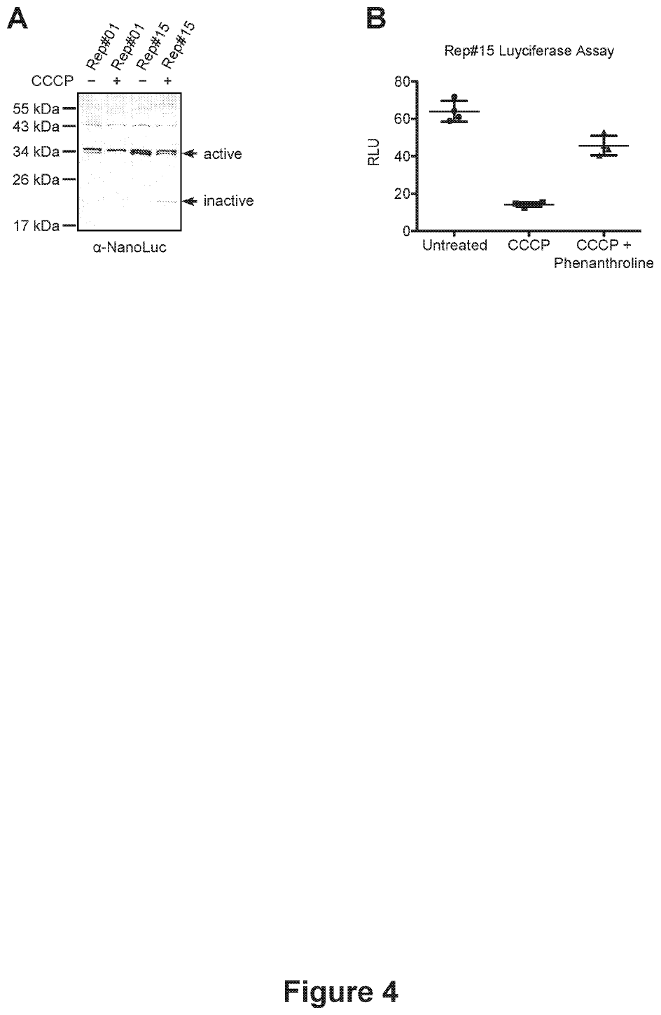

Example 2 refers to FIG. 4 and illustrates hydrolysis of reporter #01 and reporter #15. For this example, transfected cells were selected by puromycin. Reporter cells were exposed for 30 minutes to 10 μM CCCP before being harvested in RIPA buffer and subjected to 12% SDS-PAGE followed by Western-blotting. As shown in FIG. 4A, both reporter #01 and reporter #15 migrate at the same size just below the 34 kDa standard. Full-length reporter #01 has a predicted size of 39.6 kDa but was not detected in Western blots. This shows the mitochondrial import sequence of reporter #01 is efficiently cleaved, most likely by the mitochondrial processing peptidase. At the same time, reporter #15 is not processed upon import, but migrated at its expected size of 31.9 kDa. (Example 7 establishes mitochondrial import by showing co-migration of reporter #15 with OPA1 and OMA1 in mitochondria-enriched fractions.) Reporter #15 appears to be more abundant and stable, which may explain its better performance. CCCP-treatment led to processing of both reporter #01 and reporter #15. This provides additional evidence that reporter #15 is indeed localized in mitochondria where it is recognized and hydrolyzed by OMA1. It is well established in the arts, that the chelator phenanthroline can inhibit OMA1 protease in vitro (see for example Ehses et al. J Cell Biol (2009) 187(7): 1023-36; and Head et al. J Cell Biol (2009) 187(7): 959-66). Therefore, phenanthroline was used as a positive control in the reporter assay. If reporter #15 indeed is cleaved by OMA1, then phenanthroline should prevent this at least to a certain extend by inhibiting OMA1 and accordingly preserving the signal. Hek293T cells were transfected with reporter gene #15 as described in the preceding example. After 24 hours, the transfected cells were incubated with 500 μM phenanthroline in OptiMEM. After 1 hour of pretreatment, CCCP in OptiMEM/phenanthroline was added to a final concentration of 10 μM CCCP and cells incubated for another 30 minutes at 37° C. After 30 minutes, the medium was replaced with furimazine (1:100 in OptiMEM), and bioluminescence measured as described in Example 1. The result of this merely illustrative example is provided in FIG. 4B, which shows that the bioluminescence signal produced by reporter #15 is abolished when cells were treated with 10 μM CCCP, but largely preserved when cells were pretreated with 500 μM phenanthroline. This example thus demonstrates that (1) reporter #15 is cleaved indeed by OMA1 and (2) that the described method is useful for the identification of OMA1 inhibitors.

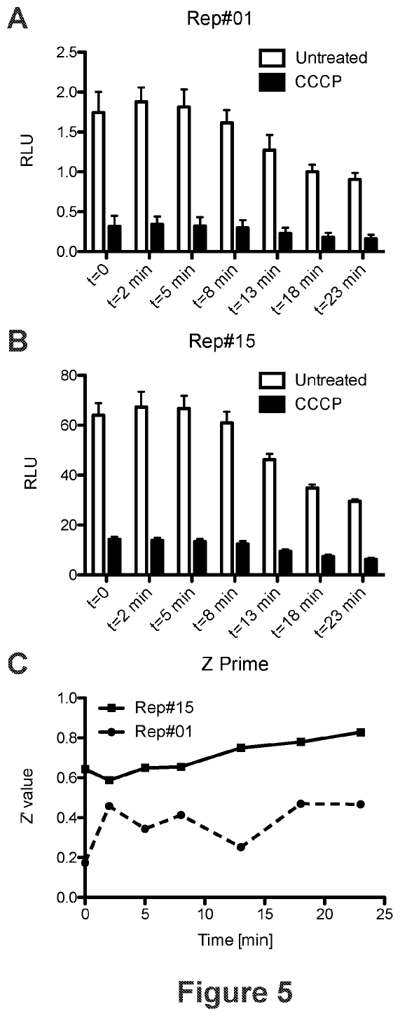

Example 3 refers to FIG. 5 and shows a time course experiment to illustrate the assay window and assay robustness. Again, Hek293T cells in a 96-well format were transfected with reporter genes #01 and #15, incubated with 10 μM CCCP 24 hours later for 30 minutes, after which the media was replaced with furimazine and bioluminescence measured at indicated time points as described in the preceding examples. As shown in FIG. 5A and B, reporter #01 and reporter #15 show comparable dynamics achieving a maximum bioluminescence signal between 2 and 5 minutes after which the signal slowly decayed over time. Robustness of an assay can be assessed and evaluated by its Z-prime score (Z′), which considers the average signal (mean) and standard deviation (S.D.) for an assay's positive and negative controls. Z′ was calculated by subtracting 3-times the sum of the standard deviations divided by the modulus of the differences of the mean from 1, whereby a Z′>0.5 is considered robust enough for high-throughput screening FIG. 5C provides the Z′-scores for this particular, nonlimiting example, which demonstrates the robustness of the reporter #15 assay for high-throughput drug screening.

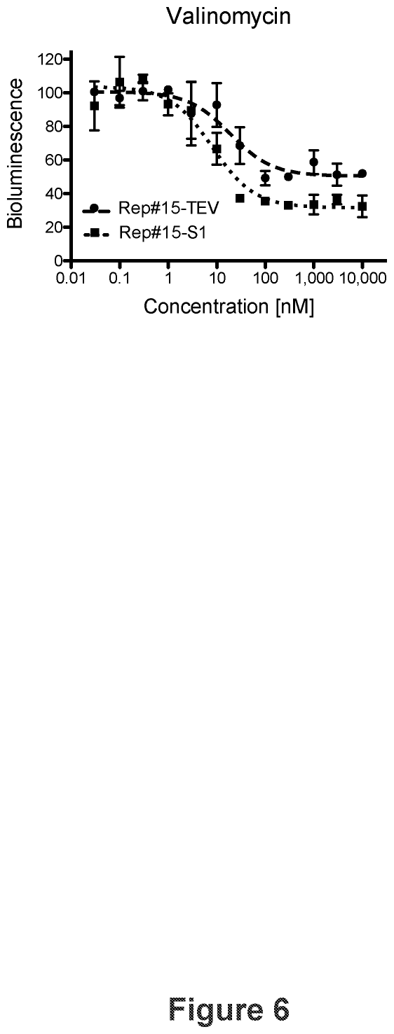

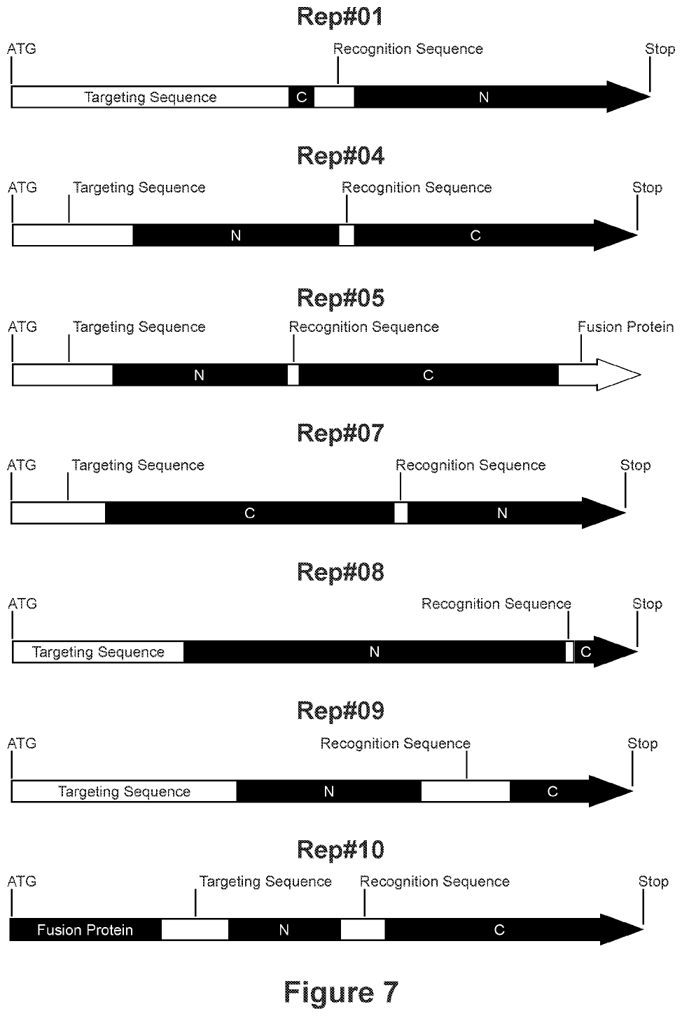

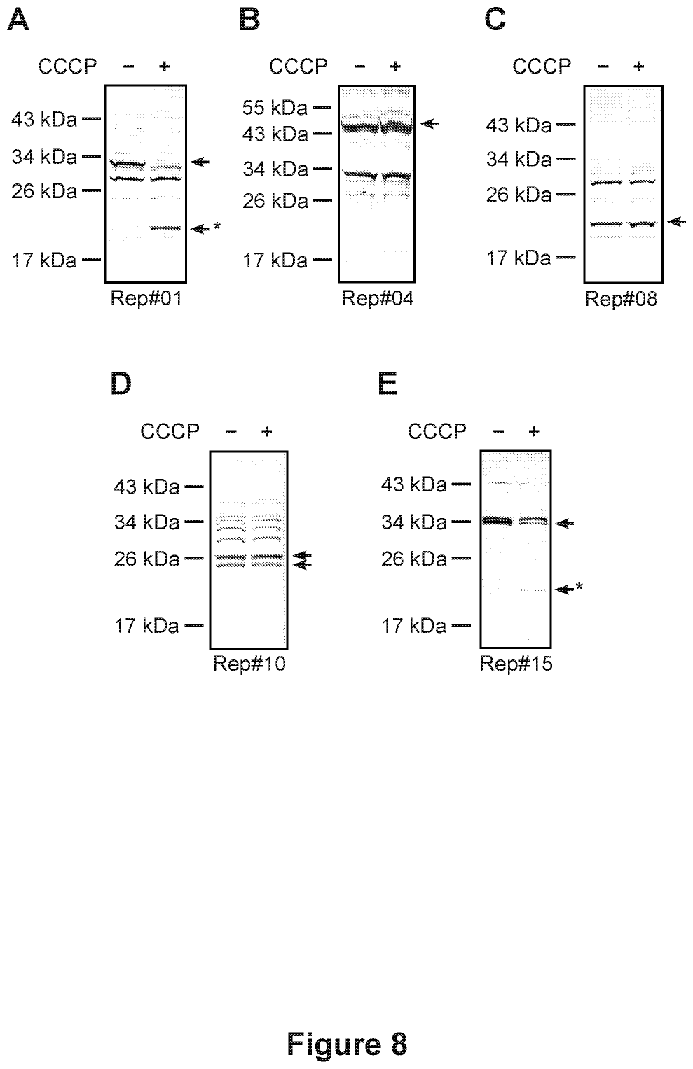

Example 4 refers to FIG. 6 and establishes that the targeting sequence alone is sufficient for recognition of the reporter by the OMA1 protease. In this example, the S1-cleavage site of reporter #15 was replaced with a TEV-cleavage site (see also Example 6). Neuro2A cells were transfected with reporter #15-S1 and reporter #15-TEV and selected by puromycin. FIG. 6 shows a valinomycin-close-response relationship for reporter #15-S1 and reporter #15-TEV, which was measured essentially as described in the preceding examples. Valinomycin can activate the OMA1 protease (see for example Ehses et al. J Cell Biol (2009) 187(7): 1023-36; and Head et al. J Cell Biol (2009) 187(7): 959-66), which can be monitored by decreasing bioluminescence of the reporter. Reporters #15-S1 and #15-TEV both showed a similar response to valinomycin with half maximal effective concentration (EC₅₀) values in the low nanomolar range. This result is surprising and stands against all teachings in the art, which claim (quite literally so) that OMA1 recognizes the S1-cleavage site (see for example U.S. Pat. No. 10,739,331B2). FIG. 7 provides an overview of the arrangement of elements of different reporter genes, which comprise inter alia the amino acid sequences of SEQ ID NO: 01 (Rep #01), SEQ ID NO: 03 (Rep #04), SEQ ID NO: 09 (Rep #08), SEQ ID NO: 13 (Rep #10), and SEQ ID NO: 15 (Rep #15). FIG. 8 shows Western blots of Hek293T cells expressing Rep #01, Rep #04, Rep #08, Rep #10, and Rep #15 incubated without or with CCCP. Hydrolysis of reporter #01 (FIG. 8A, asterisk) and reporter #15 (FIG. 8E, asterisk) in CCCP-treated cells was confirmed, while other reporter performed less well. For instance, reporter #08 (FIG. 8C) lacks parts of Rep #15's targeting sequence and has only a short recognition peptide of 4 amino acids (SEQ ID NO: 39), which appears to not be recognized by OMA1.

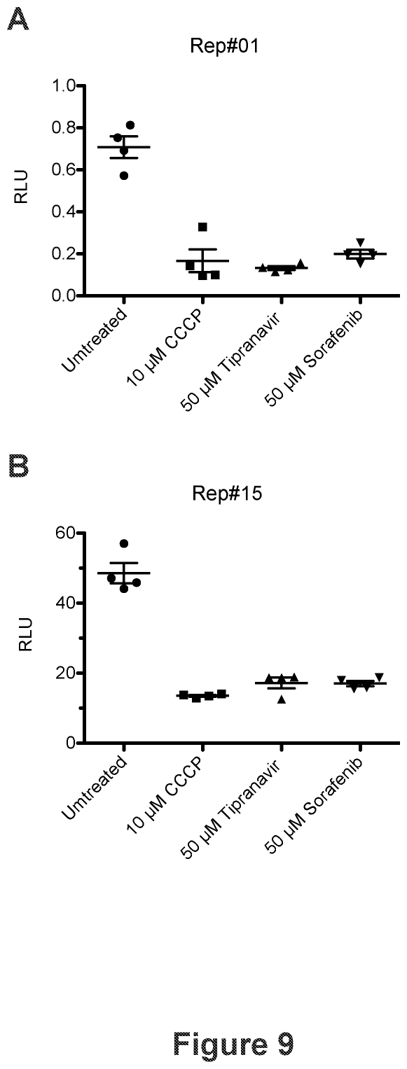

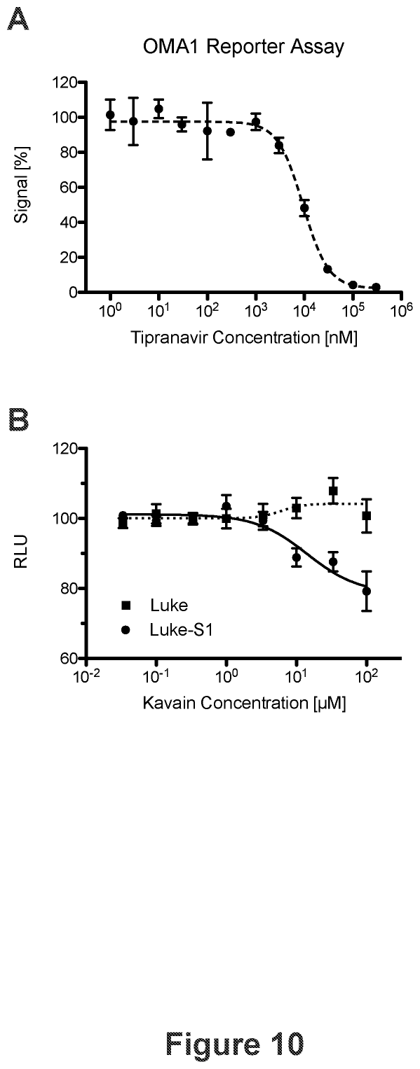

Example 5 illustrates how mitochondrial toxicity can be measured with reporter and assays of present invention. The OMA1-OPA1 pathway basically serves as a “canary-in-the-coalmine” for a mitochondrial deterioration. Mitochondria rapidly activate OMA1 when exposed to toxins, which can be monitored with the reporter and assays of present invention. Sorafenib and tipranavir are two drugs which exhibit mitochondrial toxicity (see for example the FDA labels). Hek293T cells expressing reporter #01 and #15 were exposed to CCCP, tipranavir and sorafenib (in OptiMEM) for 30 minutes before the medium was replaced with furimazine (1:100), and bioluminescence measured. The results of this merely illustrative example are provided in FIG. 9, which shows that CCCP, tipranavir and sorafenib significantly reduced bioluminescence of reporter #01 and #15. FIG. 10 shows dose-response curves for tipranavir and Kavain (CAS registry number #500-64-1), which shares structural features with tipranavir (see for example WO9530670). (A sorafenib dose-response curve is provided in FIG. 21D.) This example shows how reporter of present invention are useful for detection of mitochondrial toxicity.

The following non-limiting examples illustrate in more details the in vivo assessment of OMA1 protease activity utilizing an artificial luciferase reporter of present invention stably expressed in Hek293T cells. This reporter cell line, which is referred to as 293TR15F6 or Luke-S1, was deposited under the Budapest Treaty on Apr. 7, 2021 at the ATCC Patent Depository under the accession number PTA-I27022. They demonstrate that the engineered luciferase that incorporated a truncated portion of OPA1's amino-terminus successfully translocated to mitochondria, where it was hydrolyzed under experimental conditions under which also OPA1 was hydrolyzed. Further, they show the assays performed well in 384-well format with a Z-prime value of 0.68. The examples also illustrate two complementary drug screening approaches for OMA1 activators and for OMA1 inhibitors, respectively, which were successfully implemented in pilot screens. As already explained, OMA1 activation leads to cleavage of the reporter and deactivation of the luciferase activity. Screening of 1,280 chemically diverse molecules for compounds that would significantly lower bioluminescence resulted in 195 hits (15.2%). Furthermore, 30 of 166 approved cancer drugs (18.1%), but only 27 of 390 natural products (6.9%) activated OMA1. Considering that (i) OMA1 and OPA1 can be connected to a number of diseases, such as neurodegeneration and heart disease, and that (ii) chemotherapy-induced neuropathy and cardiotoxicity are common side-effects of many drugs, it becomes clear that the herein described assays are very useful for predicting such side-effects. Furthermore, the assays are useful for the design of better cancer therapies that (A) either avoid OMA1 activation thereby limiting adverse side-effects, or (B) that activate OMA1 specifically in malignant cells leading to apoptosis and thus inhibiting tumor growth. All these use-examples are within the scope of present invention as well as all methods and applications deduced from these. In addition, assays of present invention can be used to identify potential OMA1 inhibitors or compounds that counteract OMA1 activation as illustrated in the following examples.

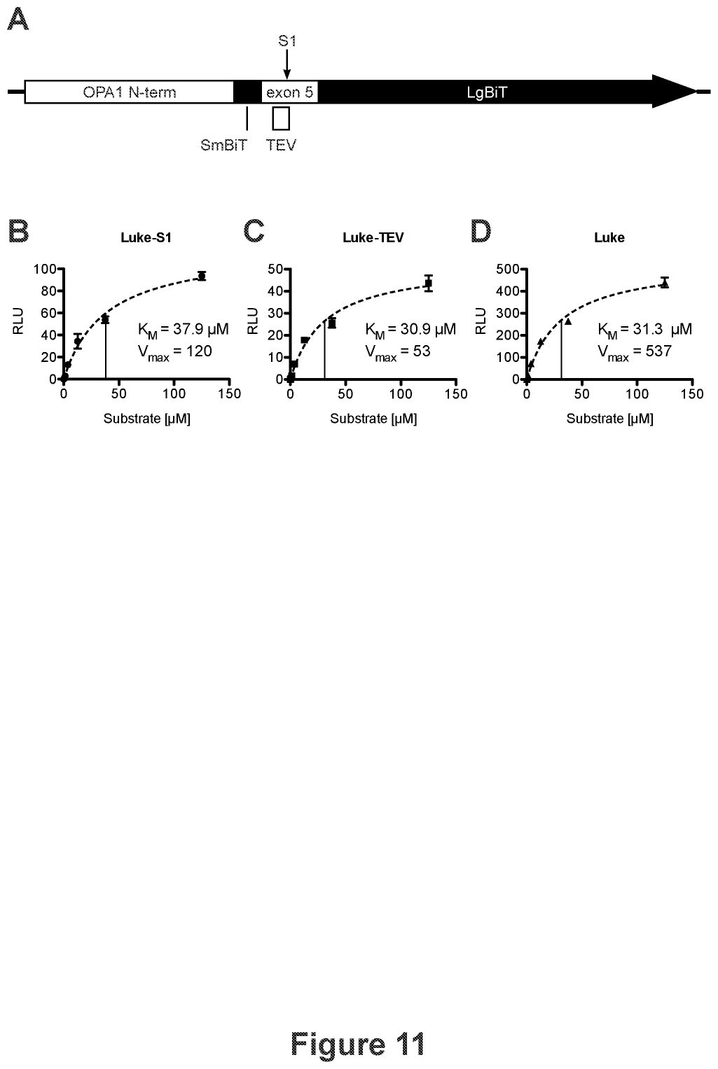

Example 6 refers to FIG. 11 and introduces the Luke-S1 reporter cell line, which is based on a modified NanoLuc complementation system (Dixon, et al. ACS Chem Biol (2016) 11(2): 400-8). NanoLuc is an engineered luciferase enzyme, which can convert the cell-permeable substrate midazopyrazinone thereby emitting light (Hall, et al. ACS Chem Biol (2012) 7(11): 1848-57). As illustrated in FIG. 11A, the Luke-S1 reporter (with the protein sequence SEQ ID NO: 15 and the DNA sequence SEQ ID NO: 16) has the last 11 n-terminal amino acids of NanoLuc (named SmBiT) c-terminally appended to the remaining 156 amino acids of the luciferase (dubbed LgBiT) via a 24-amino acid linker encoding the OPA1 S1 cleavage site with the protein sequence SEQ ID NO: 45 and the DNA sequence SEQ ID NO:46. Luke-S1 is targeted to the mitochondrial inner membrane by an 86 amino acid portion of OPA1's c-terminus with the protein sequence SEQ ID NO: 27 and the DNA sequence SEQ ID NO:28. A reporter in which the S1 site was replaced by a TEV cleavage site (referred to as ‘Luke-TEV’) and the native NanoLuc enzyme (referred to as ‘Luke’) served as controls. As illustrated in FIG. 11, Luke-S1 and Luke-TEV both assembled into a functional luciferase with Michaelis-Menten substrate affinities (K_{M}) of 37.9 μM±5.1 standard error (SE) and 30.9 μM±4.8 SE, respectively. These K_{M} values were within the range of the native NanoLuc enzyme (K_{M}[Luke]: 31.3 μM±3.7 SE). However, Vₘₐₓ was notably reduced by about an order of magnitude (Vₘₐₓ[Luke-S1]: 120 μM±6 SE; Vₘₐₓ[Luke-TEV]: 53 μM±3 SE; Vₘₐₓ[Luke]: 537 μM±23 SE).

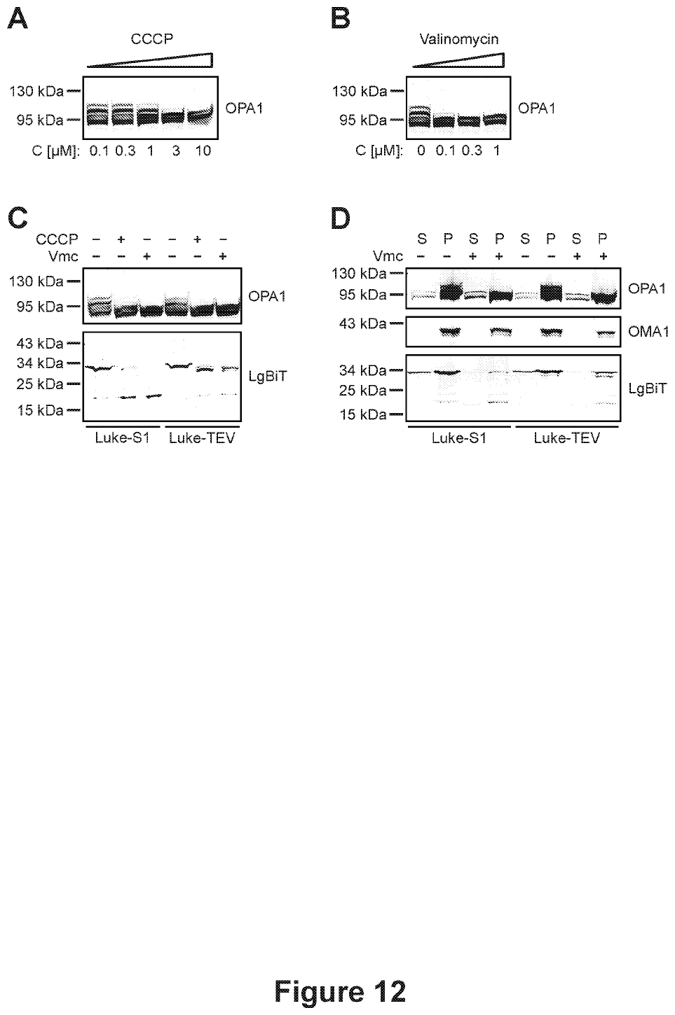

Example 7 refers to FIG. 12 and establishes that the Luke-S1 reporter and the Luke-TEV reporter were hydrolyzed under conditions that activated OMA1. In general, OMA1 shows only little activity under physiological conditions, but OMA1 cleaves OPA1 in cells treated with the protonophore CCCP or the ionophore valinomycin (see also Ehses et al. H Cell Biol (2009) 187(7): 1023-36; and Head et al. J Cell Biol (2009) 187(7): 959-66). OPA1 hydrolysis can be monitored by Western blotting. FIG. 12A shows for example the complete disappearance of L-OPA1 isoforms in Hek293T cells after 30 minutes of treatment with 3 μM CCCP. FIG. 12B shows Valinomycin was more potent in that 100 nM valinomycin sufficed for OPA1 cleavage. FIG. 12C shows that 3 μM CCCP and 100 nM valinomycin also induced cleavage of the Luke-S1 reporter in Western blots. The LgBiT antibody recognized a protein in untreated Luke-S1 cells migrating just below the 34 kDa standard. The predicted size of full-length Luke-S1 is 31.8 kDa. In cells treated with CCCP and with valinomycin, this band leveled off and a band migrating above the 15 kDa standard became much more prominent, which according to its approximate size of about 19 kDa corresponds to the reporter hydrolyzed at the S1 site. Surprisingly, also Luke-TEV was cleaved upon CCCP or valinomycin treatment, but showed a different cleavage pattern with only a minor size reduction (FIG. 12C). Remarkably, the inner-membrane anchor sufficed for the recognition by OMA1. The fact that Luke-TEV is also cleaved demonstrates that OMA1 is promiscuous in its substrate recognition and that the OPA1 S1 cleavage site is not necessary for the design of an OMA assay. This is against the teachings in the art (see for example U.S. Pat. No. 10,739,331B2). FIG. 12D shows a cell fractionation by differential centrifugation with Luke-S1 and Luke-TEV in mitochondria-enriched fractions. The full-length Luke-S1 reporter was detected in mitochondria-enriched fractions together with OPA1 and OMA1 (FIG. 12D). Also Luke-TEV emigrated with OPA1 and OMA1 following differential centrifugation. Both cleavage products S-OPA1 and LgBiT in valinomycin-treated cells were not actively released from mitochondria but were still mainly present in the mitochondria-enriched fractions. This notion also establishes that the integrity of mitochondria was not impacted by the cell fractionation procedure and that the mitochondrial outer membrane remained intact.

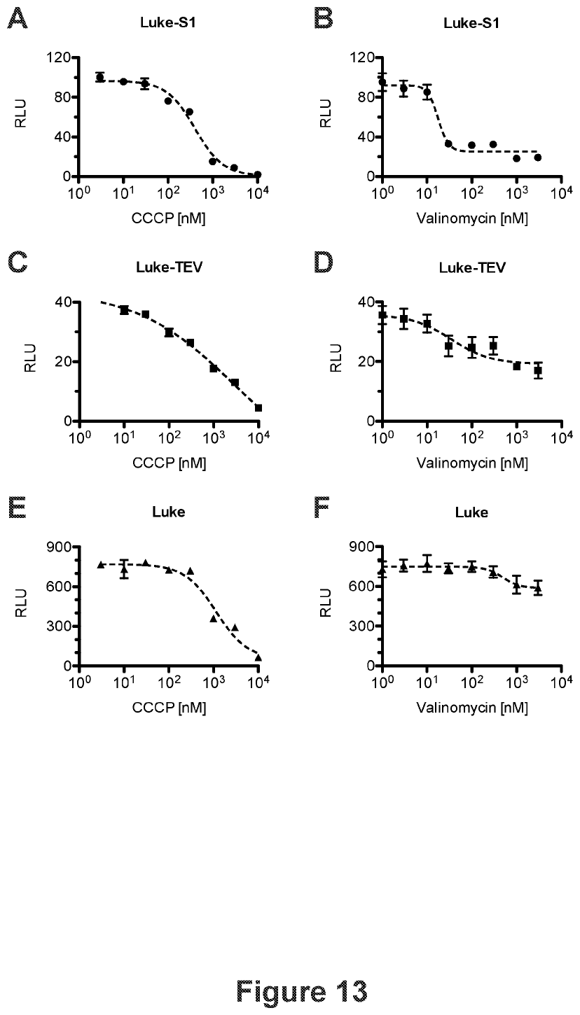

Example 8 refers to FIG. 13 and shows the in vivo protease assays' response to CCCP and valinomycin. The half maximal effective concentration (EC₅₀) for Luke-S1 reporter cells incubated for 30 minutes with increasing CCCP concentrations was 398.4 nM (95% confidence interval: 291.3 to 545.0 nM; FIG. 13A). Dose-response relationships for valinomycin demonstrated an EC₅₀ of 17.6 nM (95% confidence interval: 12.2 to 25.4 nM; FIG. 13B). CCCP had also an effect on Luke-TEV with an EC₅₀ of 566.4 nM (95% confidence interval: 352.2 to 910.7 nM; FIG. 13C), while valinomycin produced only a minor signal reduction (FIG. 13D). This shows that Luke-TEV is most likely cleaved close to its amino-terminus, because valinomycin did not diminish its luciferase activity at large, further highlighting the difficulties and challenges of designing an OMA1 assay that actually works. Interestingly, CCCP completely eliminated Luke's bioluminescence with an EC₅₀ of 967.9 nM (95% confidence interval: 793.5 to 1,898 nM; FIG. 13E). At the same time, valinomycin had no effects (FIG. 13F). CCCP's assay interference with NanoLuc-based reporter systems was previously noted and is the most likely explanation for this observation (Pereira et al. J Mol Biol (2019) 431(8): 1689-1699).

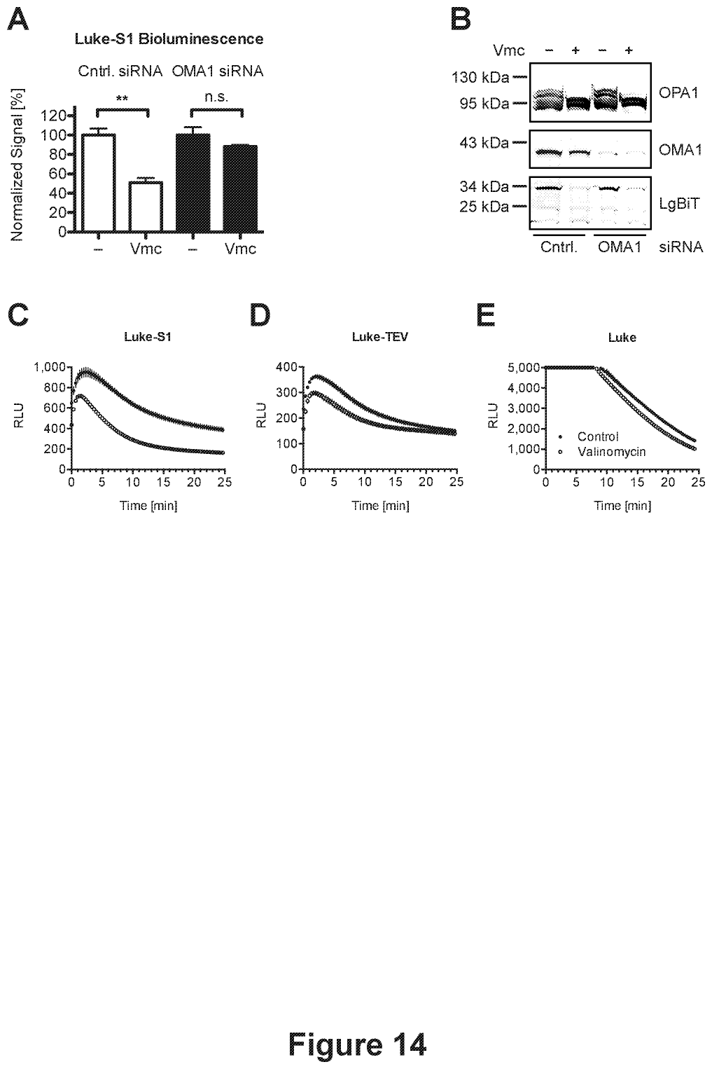

Example 9 refers to FIG. 14 and provides supporting data that establish specificity of Luke-S1 and shows its temporal resolution. As illustrated in the preceding examples, valinomycin leads to significant signal reduction of Luke-S1 due to cleavage of the reporter. This was also the case in Luke-S1 cells transfected with a control siRNA and treated with 100 nM valinomycin for 30 minutes. In contrast, Luke-S1 cells transfected with OMA1 siRNA showed no significant signal reduction when treated with valinomycin (FIG. 14A, one-way ANOVA for multigroup comparison: p=0.003). siRNA-mediated OMA1 knock-down reduced protein levels by about 70% in these experiments (FIG. 14B). This data provides further evidence that the Luke-S1 reporter is recognized and cleaved by the OMA1 protease. Exposing reporter cells simultaneously to valinomycin and luciferase substrate and immediately recording bioluminescence in real-time helped to better understand the dynamic nature of the OMA1 protease and the temporal resolution of the reporter, Luke-S1 showed a marked signal reduction from the start of the measurement compared to cells without valinomycin (FIG. 14C). The signal also declined more rapidly in the presence of valinomycin during the first 15 minutes before the signal stabilized at a significantly lower intensity (FIG. 14C). Luke-TEV on the other hand showed a much smaller difference from the beginning of the measurement and after about 15 minutes there was no difference between cells with or without valinomycin anymore (FIG. 14D). The signal intensity of Luke saturated the photomultiplier tube of the instrument in the beginning. Nonetheless, after 10 minutes there were no notable differences in bioluminescence levels or signal decay between the two treatment conditions (FIG. 14E). (The rapid and continued signal decay observed in Luke cells was most likely due to substrate depletion.) Taken together, these findings show (i) OMA1 is rapidly activated by valinomycin and (ii) the Luke-S1 reporter has a high dynamic range and quickly responds to OMA1 activation.

Example 10 provides a merely illustrative example of a drug screening campaign of 1,280 chemically diverse compounds for OMA1 activators. For this drug screen, Luke-S1 cells were incubated with 10 μM test compounds for 60 minutes in 384-well plates prior to the addition of luciferase substrate. Valinomycin-treated cells in columns #2 and #23 of each plate served as positive controls to which all measurements were normalized (see FIG. 15A for a snapshot of a 384-well plate of this screen). The average bioluminescence of untreated cells in this assay was 372.1%±24.4 standard deviation (SD) when normalized to valinomycin-treated cells (100%±4.9 SD; FIG. 15B). The calculated Z-prime value was 0.68. The average standard deviation of 128 controls across all plates was 100%±14.5 SD (FIG. 15C). The higher variability of the actual screen was in part ascribed to plate drift over the about 5 minutes time it required to measure each plate (see also FIG. 15D). This screen searched for molecules that would activate OMA1 in a comparable manner to valinomycin. For this reason, the hit-threshold was defined as a reduced bioluminescence that would fall within 3 standard deviations of the valinomycin-treated controls (<143.5%; FIG. 15C & D, dotted line). 195 of 1,280 test molecules (15.2%) lowered the signal below the 143.5%-threshold (FIG. 15C % D). This assay would also pick up any chemicals that interfere with the luciferase enzyme itself. 26 of the 195 hits suppressed bioluminescence even below the lower 3×SD threshold of 56.5%. These 26 molecules (2.0%) presumably inhibited the luciferase rather than engage OMA1. An independent screening campaign found that 2.7% of the 42,000 tested chemicals inhibited the NanoLuc enzyme by at least 30% (Ho et al. ACS Chem Biol (2013) 8(5): 1009-17).

Example 11 provides a merely illustrative example of a drug screening campaign of 3,520 chemically diverse compounds for OMA1 inhibitors. For this screen, Luke-S1 cells were preincubated for 1-2 hours with 10 μM test compounds before valinomycin was added (100 nM final concentration) for another 30 minutes. The goal of this screen was to identify compounds that would counteract valinomycin-induced OMA1 activation. For this reason, untreated cells served as controls to which all measurements were normalized (see FIG. 16A for a snapshot of a 384-well plate of this screen). The average of the 352 controls across 11 plates was 100%±10.4 SD. Test molecules that would sustain bioluminescence in valinomycin-treated Luke-S1 cells to a level within 3×SD of untreated cells were considered hits. The hit threshold was set accordingly at >68.8% (FIG. 16B & C, dotted line). The average signal of the 3,520 test molecules after the addition of valinomycin was 30.7%±7.0 SD. 26 molecules (0.7%) quenched bioluminescence below the lower 3×SD threshold of 9.8% most likely by interfering with NanoLuc. One test compound had a signal of 71.4%, which was within three standard deviations of untreated cells. However, retesting of this molecule at six different concentrations could not confirm the alleged hit (FIG. 16D).

Example 12 refers to FIGS. 17 to 21 and illustrates broad OMA1 activation by cancer drugs from different classes. It is known that cancer drugs, such as cisplatin and sorafenib, can promote OPA cleavage (see for example Zhao et al. Lab Invest (2013) 93(1): 8-19; Kong et al. J Biol Chem (2014) 289(39): 27134-45). Survival data of individuals with cancer are also significantly correlated with OMA1 gene expression levels (see U.S. Pat. No. 10,906,931B2). Yet, quite surprisingly, a large number of FDA-approved cancer drugs 30 of 166 cancer drugs (18.1%) from different classes triggered OMA1 activation in Luke-S1 assays, while for example only 27 of 390 natural products (6.9%) activated OMA1. Luke-S1 reporter cells were incubated with 10 μM of drugs for 60 minutes after which luciferase substrate was added and bioluminescence measured. The signal intensity for this study was normalized to untreated cells (100%±12.5 SD; valinomycin treated controls: 18.5%±5.6 SD) and the significance level was defined as signal drop by at least 3 standard deviations from untreated controls (>37.5% reduction). As provided in FIG. 17, 30 of 166 approved cancer drugs (18.1%) reduced bioluminescence by 37.5% to 85.6%. 18 of these were kinase inhibitors (60.0%), which constituted only 39.2% of all the drugs in this collection (65 of 166). This over representation was statistically significant (Fisher's exact test: p=0.013). Kinase inhibitors are notorious for cardiotoxicity in the clinic and different methods are being developed for the preclinical evaluation and prediction of cardiotoxicity (see for example Sharma et al. Sci Transl Med (2017) 9(377): eaaf2584). It is known in the arts that OPA1 mutations as well as conditional YME1L1 knock-out impaired cardiac function in mice (see for example Chen et al. J Am Heart Assoc (2012) 1(5): e00301; Piquereau et al. Cardiovasc Res (2012) 94(3): 408-17; Wai et al. Science (2015) 350(6265): aad0116; Le Page et al. PLoS One (2016) 11(10): e0164066). OMA1 ablation on the other hand could protect cardiomyocytes in 3 different mouse models for heart failure (see Acin-Perez et al. Sci Transl Med (2018) 10(434): eaan4935). And a missense variant in human OMA1 increased the mortality risk of heart failure in two cohorts with 559 and 999 individuals (see Hu et al. Cardiovasc Drugs Ther (2020) 34(3): 345-356). Cardiotoxicity prompted research into cross-reactivity of multitargeted tyrosine kinase inhibitors with mitochondria before (Will et al. Toxicol Sci (2008) 106(1): 153-61). Putting all these different pieces of data together with the finding of abundant OMA1 activation by kinase inhibitors (among others), it becomes clear that kinase inhibitors may indeed interact with the OMA1 mechanism resulting in cardiotoxicity in individuals taking these drugs. Within this conceptual framework Luke-S1 assays present a straight-forward and economic way of testing for unwanted cytotoxicity at scale. Furthermore, any and each of the drugs and molecules described herein may be used to modulate OMA1 activity in a subject with a disease amenable to OMA1 modulatory therapies, such a disease being readily known in the arts and may comprise inter alia a disease disclosed in U.S. Pat. No. 10,906,931B2.

Without further elaboration, it is believed that one skilled in the art can, using the preceding description and examples, utilize the present invention to its fullest extent. The preceding preferred specific embodiments are, therefore, to be construed as merely illustrative, and not limitative of present disclosure in any way whatsoever. Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated (again) by reference in its entirety to the same extent as if each reference was individually incorporated by reference. Where a conflict exists between the instant application and a reference provided herein, the instant application shall dominate.

1-150. (canceled)

151. A reporter for measuring protease activity, said reporter operatively combining functional elements selected from a targeting sequence, an entity or fragment “N” of an enzymatic moiety or protein domain, an entity or fragment “C” of an enzymatic moiety or protein domain, which can complement “N” in a way that produces a signal, and a sequence-motif that can be recognized by the OMA1 protease, wherein a reduced signal indicates an increased OMA1 activity.

152. The reporter for measuring OMA1 protease activity of the preceding claim 151, wherein the targeting sequence has at least 75% identity with SEQ ID NO: 17, SEQ ID NO: 19, SEQ ID NO: 21, SEQ ID NO: 23, SEQ ID NO: 25, or SEQ ID NO: 27 or variations or combinations thereof.

153. The reporter for measuring OMA1 protease activity of the preceding claim 151, wherein entity or fragment “N” has at least 75% identity with SEQ ID NO: 49, SEQ ID NO: 51, SEQ ID NO: 53, SEQ ID NO: 55, or SEQ ID NO: 59 or variations or combinations thereof, provided that fragment “C” has at least 75% identity with SEQ ID NO: 61, SEQ ID NO: 63, SEQ ID NO: 65, SEQ ID NO: 69, or SEQ ID NO: 73 or variations or combinations thereof.

154. The reporter for measuring OMA1 protease activity of the preceding claim 151, wherein the sequence-motif that can be recognized by the OMA1 protease has at least 75% identity with SEQ ID NO: 33, SEQ ID NO: 35, SEQ ID NO: 37, SEQ ID NO: 39, SEQ ID NO: 41, SEQ ID NO: 43, or SEQ ID NO: 45 or variations or combinations thereof.

155. The reporter for measuring OMA1 protease activity of the preceding claim 151, wherein the targeting sequence is also the sequence recognized by OMA1.

156. The reporter for measuring OMA1 protease activity of the preceding claim 151, wherein the targeting sequence is 30 or more amino acids, preferably 80 amino acids, but not more than 160 amino acids.

157. A recombinant expression vector comprising the reporter for measuring OMA1 protease activity of the preceding claim 151.

158. A recombinant host cell comprising the reporter for measuring OMA1 protease activity of the preceding claim 151.

159. A kit comprising the reporter for measuring OMA1 protease activity of the preceding claim 151.

160. A method for predicting mitochondrial toxicity comprising the reporter for measuring OMA1 protease activity of the preceding claim 151.

161. A method for predicting adverse events comprising the reporter for measuring OMA1 protease activity of the preceding claim 151.

162. A method for detecting protease activity comprising the reporter for measuring OMA1 protease activity of the preceding claim 151.

163. A method of detecting a protease activity in a sample comprising
a. combining the sample with the reporter for measuring OMA1 protease activity of the preceding claim 151,
b. measuring a signal, and
c. comparing a value of the signal with a value of a signal from a control,
wherein the signal is inversely correlated to the protease activity.

164. A method of identifying OMA1 protease inhibitors comprising
a. combining a molecule with a reporter comprising a functional moiety separated by an OMA1 cleavage site,
b. activating OMA1 protease,
c. measuring a signal,
d. and selecting a molecule, which sustains the signal compared to a control without molecule.

165. A method of predicting in vivo toxicity of a molecule, said method comprising the steps of
a. combining the molecule with a recombinant host expressing a reporter comprising a fragment X of a signal-producing protein separated by a recognition element from a fragment Y of a signal-producing protein, which complements the fragment X in a way that a signal is emitted, and
b. detecting cleavage of the recognition element as a change in the signal emitted by fragments X and Y,
wherein the detection of increased cleavage indicates increased toxicity of the molecule.


SEQUENCE LISTING (76 sequences)

SEQ ID NO: 1 - protein, 346 aa, Artificial Sequence
  Synthetic Construct
MWGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFEKIRKALPSSEDLVKLAPDFDKIVVTGYRLFEEILSGSPEETAFRATDRGSESDKHFRKVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 2 - DNA, 1041 bp, Artificial Sequence
  Synthetic sequence
atgtggggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttgagaaaattagaaaagcccttcctagttcagaagaccttgtaaagttagcaccagactttgacaagattgttgtgaccggctaccggctgttcgaggagattctgtcaggttctccggaagaaacggcgtttagagcaacagatcgtggatctgaaagtgacaagcattttagaaaggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagctaa

SEQ ID NO: 3 - protein, 514 aa, Artificial Sequence
  Synthetic sequence
MWGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAPEETAFRATDRGVSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 4 - DNA, 1545 bp, Artificial Sequence
  Synthetic sequence
atgtggggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgccggaagaaacggcgtttagagcaacagatcgtggagtgagcaagggcgaggagctgatcaaggagaacatgagaagcaagctgtacctggaaggcagcgtgaacggccaccagttcaagtgcacccacgaaggggagggcaagccctacgagggcaagcagaccaacaggatcaaggtggtggagggaggccccctgccgttcgcattcgacatcctggccacccactttatgtacgggagcaaggtgttcatcaagtaccccgccgacctccccgattattttaagcagtccttccctgagggcttcacatgggagagagtcatggtgttcgaagacgggggcgtgctgaccgccacccaggacaccagcctccaggacggcgagctcatctacaacgtcaaggtcagaggggtgaacttcccagccaacggccccgtgatgcagaagaaaacactgggctgggagcccagcaccgagaccatgtaccccgctgacggcggcctggaaggcagatgcgacaaggccctgaagctcgtgggcgggggccatctgcacgtcaacttcaagaccacatacaagtccaagaaacccgtgaagatgcccggcgtccactacgtggaccgcagactggaaagaatcaaggaggccgacaacgagacctacgtcgagcagtacgagcacgctgtggccagatactccaacctgggcggaggcatggacgagctgtacaagtaa

SEQ ID NO: 5 - protein, 578 aa, Artificial Sequence
  Synthetic sequence
MWGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAPEETAFRATDRGGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 6 - DNA, 1737 bp, Artificial Sequence
  Synthetic sequence
atgtggggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgccggaagaaacggcgtttagagcaacagatcgtggaggatccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggttaa

SEQ ID NO: 7 - protein, 658 aa, Artificial Sequence
  Synthetic sequence
MWGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKASPEETAFRATDRGSESAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPV

SEQ ID NO: 8 - DNA, 1977 bp, Artificial Sequence
  Synthetic sequence
atgtggggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacactggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaaggctagcccggaagaaacggcgtttagagcaacagatcgtggatctgaatctgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgtttaa

SEQ ID NO: 9 - protein, 237 aa, Artificial Sequence
  Synthetic sequence
MWGIKGSLPLQKLHLVSRSIYHSHHPTLKLQRPQLRTSFQQFSSLTNLPLRKLKFSPIKYGYQPRRNVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLAFRATINGVTGWRLCERILA

SEQ ID NO: 10 - DNA, 714 bp, Artificial Sequence
  Synthetic sequence
atgtggggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctggcgttccgagcaaccatcaacggagtgaccggctggcggctgtgcgaacgcattctggcgtaa

SEQ ID NO: 11 - protein, 284 aa, Artificial Sequence
  Synthetic sequence
MWGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMVEKFVGTWKIADSHNFGEYLKAIGAPKELSDGGDATTPTLYISQKDGDKMTVKIENGPPTFLDTQVKFKLGEEFDEFPSDRRKEYIDFGSPEETAFRATDRGSESDKHFRKGLLGELILLQQQKGVKSVVNLVGEKLVYVQKWDGKETTYVREIKDGKLVVTLTMGDVVAVRSYRRATE

SEQ ID NO: 12 - DNA, 852 bp, Artificial Sequence
  Synthetic sequence
atgtggggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatggttgagaagtttgttggcacttggaagatagctgatagtcataactttggagaatacctgaaggccattggtgcaccgaaagaactttctgatggcggagacgcaacaactcccacactctatatcagccaaaaggacggggacaaaatgacagtaaagatcgaaaacggaccacccactttcctggatacgcaggtgaagtttaagctcggagaggagttcgatgaatttcccagcgaccgccggaaagagtacatcgacttcggcagccccgaggagaccgccttcagggccaccgacaggggcagcgagagcgacaagcacttcaggaagggcctgctgggcgagctgatcctgctgcagcagcagaaaggcgtgaagagcgtagtgaacttagttggagaaaaactggtctatgttcagaaatgggacggtaaggagaccacttatgtgcgagaaattaaggacggaaagcttgtggtgacgctgactatgggagacgtcgtagcagtgcggagttatagacgcgcgaccgaa

SEQ ID NO: 13 - protein, 286 aa, Artificial Sequence
  Synthetic sequence
MWGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFVFTLEDFVGDWRQTAGYNLDQVLEQGGVSSLFQNLGVSVTPIQRIVLSGSPEETAFRATDRGSESDKHFRKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 14 - DNA, 861 bp, Artificial Sequence
  Synthetic sequence
atgtggggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggttctccggaagaaacggcgtttagagcaacagatcgtggatctgaaagtgacaagcattttagaaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 15 - protein, 280 aa, Artificial Sequence
  Synthetic sequence
MFWPARLATRLLKLRYLILGSAVGGGYTAKKTFDQWKDMIPDLSEYKWIVPDIVWEIDEYIDFEKIRKALPSSEDLVKLAPDFDKIVVTGYRLFEEILSGSPEETAFRATDRGSESDKHFRKVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 16 - DNA, 843 bp, Artificial Sequence
  Synthetic sequence
atgttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttgagaaaattagaaaagcccttcctagttcagaagaccttgtaaagttagcaccagactttgacaagattgttgtgaccggctaccggctgttcgaggagattctgtcaggttctccggaagaaacggcgtttagagcaacagatcgtggatctgaaagtgacaagcattttagaaaggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagctaa

SEQ ID NO: 17 - protein, 152 aa, Artificial Sequence
  Synthetic sequence
WGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFEKIRKALPSSEDLVKLAPDFDKIV

SEQ ID NO: 18 - DNA, 456 bp, Artificial Sequence
  Synthetic sequence
tggggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttgagaaaattagaaaagcccttcctagttcagaagaccttgtaaagttagcaccagactttgacaagattgtt

SEQ ID NO: 19 - protein, 98 aa, Artificial Sequence
  Synthetic sequence
WGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTF

SEQ ID NO: 20 - DNA, 294 bp, Artificial Sequence
  Synthetic sequence
tggggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagactttt

SEQ ID NO: 21 - protein, 66 aa, Artificial Sequence
  Synthetic sequence
WGIKGSLPLQKLHLVSRSIYHSHHPTLKLQRPQLRTSFQQFSSLTNLPLRKLKFSPIKYGYQPRRN

SEQ ID NO: 22 - DNA, 198 bp, Artificial Sequence
  Synthetic sequence
tggggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaat

SEQ ID NO: 23 - protein, 103 aa, Artificial Sequence
  Synthetic sequence
WGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKD

SEQ ID NO: 24 - DNA, 309 bp, Artificial Sequence
  Synthetic sequence
tggggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagat

SEQ ID NO: 25 - protein, 32 aa, Artificial Sequence
  Synthetic sequence
FWPARLATRLLKLRYLILGSAVGGGYTAKKTF

SEQ ID NO: 26 - DNA, 96 bp, Artificial Sequence
  Synthetic sequence
ttttggccagcaagattagctacgagactcttaaaacttcgctatctcatactaggatcggctgttgggggtggctacacagccaaaaagactttt

SEQ ID NO: 27 - protein, 86 aa, Artificial Sequence
  Synthetic sequence
FWPARLATRLLKLRYLILGSAVGGGYTAKKTFDQWKDMIPDLSEYKWIVPDIVWEIDEYIDFEKIRKALPSSEDLVKLAPDFDKIV

SEQ ID NO: 28 - DNA, 258 bp, Artificial Sequence
  Synthetic sequence
ttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttgagaaaattagaaaagcccttcctagttcagaagaccttgtaaagttagcaccagactttgacaagattgtt

SEQ ID NO: 29 - protein, 20 aa, Artificial Sequence
  Synthetic sequence
RLRRAAVACEVCQSLVKHSS

SEQ ID NO: 30 - DNA, 60 bp, Artificial Sequence
  Synthetic sequence
cgactacgtcgggccgctgtggcctgtgaggtctgccagtctttagtgaaacacagctct

SEQ ID NO: 31 - protein, 86 aa, Artificial Sequence
  Synthetic sequence
WRLRRAAVACEVCQSLVKHSSGIKGSLPLQKLHLVSRSIYHSHHPTLKLQRPQLRTSFQQFSSLTNLPLRKLKFSPIKYGYQPRRN

SEQ ID NO: 32 - DNA, 258 bp, Artificial Sequence
  Synthetic sequence
tggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaat

SEQ ID NO: 33 - protein, 23 aa, Artificial Sequence
  Synthetic sequence
GSPEETAFRATDRGSESDKHFRK

SEQ ID NO: 34 - DNA, 69 bp, Artificial Sequence
  Synthetic sequence
ggttctccggaagaaacggcgtttagagcaacagatcgtggatctgaaagtgacaagcattttagaaag

SEQ ID NO: 35 - protein, 12 aa, Artificial Sequence
  Synthetic sequence
PEETAFRATDRG

SEQ ID NO: 36 - DNA, 36 bp, Artificial Sequence
  Synthetic sequence
ccggaagaaacggcgtttagagcaacagatcgtgga

SEQ ID NO: 37 - protein, 14 aa, Artificial Sequence
  Synthetic sequence
PEETAFRATDRGSE

SEQ ID NO: 38 - DNA, 42 bp, Artificial Sequence
  Synthetic sequence
ccggaagaaacggcgtttagagcaacagatcgtggatctgaa

SEQ ID NO: 39 - protein, 4 aa, Artificial Sequence
  Synthetic sequence
AFRA

SEQ ID NO: 40 - DNA, 12 bp, Artificial Sequence
  Synthetic sequence
gcgtttagagca

SEQ ID NO: 41 - protein, 40 aa, Artificial Sequence
  Synthetic sequence
EYIDFGSPEETAFRATDRGSESDKHFRKGLLGELILLQQQ

SEQ ID NO: 42 - DNA, 120 bp, Artificial Sequence
  Synthetic sequence
gagtacatcgacttcggcagccccgaggagaccgccttcagggccaccgacaggggcagcgagagcgacaagcacttcaggaagggcctgctgggcgagctgatcctgctgcagcagcag

SEQ ID NO: 43 - protein, 21 aa, Artificial Sequence
  Synthetic sequence
SPEETAFRATDRGSESDKHFR

SEQ ID NO: 44 - DNA, 63 bp, Artificial Sequence
  Synthetic sequence
tctccggaagaaacggcgtttagagcaacagatcgtggatctgaaagtgacaagcattttaga

SEQ ID NO: 45 - protein, 24 aa, Artificial Sequence
  Synthetic sequence
SGSPEETAFRATDRGSESDKHFRK

SEQ ID NO: 46 - DNA, 72 bp, Artificial Sequence
  Synthetic sequence
tcaggttctccggaagaaacggcgtttagagcaacagatcgtggatctgaaagtgacaagcattttagaaag

SEQ ID NO: 47 - protein, 24 aa, Artificial Sequence
  Synthetic sequence
SGSTTENLYFQSDNGSESDKHFRK

SEQ ID NO: 48 - DNA, 72 bp, Artificial Sequence
  Synthetic sequence
tcaggttctacaaccgagaacctgtacttccagagcgacaacggatctgaaagtgacaagcattttagaaag

SEQ ID NO: 49 - protein, 158 aa, Artificial Sequence
  Synthetic sequence
VFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 50 - DNA, 474 bp, Artificial Sequence
  Synthetic sequence
gtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagc

SEQ ID NO: 51 - protein, 170 aa, Artificial Sequence
  Synthetic sequence
VFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 52 - DNA, 510 bp, Artificial Sequence
  Synthetic sequence
gtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcg

SEQ ID NO: 53 - protein, 232 aa, Artificial Sequence
  Synthetic sequence
SAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPV

SEQ ID NO: 54 - DNA, 699 bp, Artificial Sequence
  Synthetic sequence
tctgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgtttaa

SEQ ID NO: 55 - protein, 150 aa, Artificial Sequence
  Synthetic sequence
VFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSL

SEQ ID NO: 56 - DNA, 450 bp, Artificial Sequence
  Synthetic sequence
gtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctg

SEQ ID NO: 57 - protein, 84 aa, Artificial Sequence
  Synthetic sequence
MVEKFVGTWKIADSHNFGEYLKAIGAPKELSDGGDATTPTLYISQKDGDKMTVKIENGPPTFLDTQVKFKLGEEFDEFPSDRRK

SEQ ID NO: 58 - DNA, 252 bp, Artificial Sequence
  Synthetic sequence
atggttgagaagtttgttggcacttggaagatagctgatagtcataactttggagaatacctgaaggccattggtgcaccgaaagaactttctgatggcggagacgcaacaactcccacactctatatcagccaaaaggacggggacaaaatgacagtaaagatcgaaaacggaccacccactttcctggatacgcaggtgaagtttaagctcggagaggagttcgatgaatttcccagcgaccgccggaaa

SEQ ID NO: 59 - protein, 49 aa, Artificial Sequence
  Synthetic sequence
VFTLEDFVGDWRQTAGYNLDQVLEQGGVSSLFQNLGVSVTPIQRIVLSG

SEQ ID NO: 60 - DNA, 147 bp, Artificial Sequence
  Synthetic sequence
gtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggt

SEQ ID NO: 61 - protein, 12 aa, Artificial Sequence
  Synthetic sequence
VTGYRLFEEILS

SEQ ID NO: 62 - DNA, 36 bp, Artificial Sequence
  Synthetic sequence
gtgaccggctaccggctgttcgaggagattctgtca

SEQ ID NO: 63 - protein, 233 aa, Artificial Sequence
  Synthetic sequence
VSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 64 - DNA, 699 bp, Artificial Sequence
  Synthetic sequence
gtgagcaagggcgaggagctgatcaaggagaacatgagaagcaagctgtacctggaaggcagcgtgaacggccaccagttcaagtgcacccacgaaggggagggcaagccctacgagggcaagcagaccaacaggatcaaggtggtggagggaggccccctgccgttcgcattcgacatcctggccacccactttatgtacgggagcaaggtgttcatcaagtaccccgccgacctccccgattattttaagcagtccttccctgagggcttcacatgggagagagtcatggtgttcgaagacgggggcgtgctgaccgccacccaggacaccagcctccaggacggcgagctcatctacaacgtcaaggtcagaggggtgaacttcccagccaacggccccgtgatgcagaagaaaacactgggctgggagcccagcaccgagaccatgtaccccgctgacggcggcctggaaggcagatgcgacaaggccctgaagctcgtgggcgggggccatctgcacgtcaacttcaagaccacatacaagtccaagaaacccgtgaagatgcccggcgtccactacgtggaccgcagactggaaagaatcaaggaggccgacaacgagacctacgtcgagcagtacgagcacgctgtggccagatactccaacctgggcggaggcatggacgagctgtacaag

SEQ ID NO: 65 - protein, 297 aa, Artificial Sequence
  Synthetic sequence
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 66 - DNA, 891 bp, Artificial Sequence
  Synthetic sequence
ggatccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggt

SEQ ID NO: 67 - protein, 313 aa, Artificial Sequence
  Synthetic sequence
DTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKAS

SEQ ID NO: 68 - DNA, 939 bp, Artificial Sequence
  Synthetic sequence
gacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacactggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaaggctagc

SEQ ID NO: 69 - protein, 16 aa, Artificial Sequence
  Synthetic sequence
TINGVTGWRLCERILA

SEQ ID NO: 70 - DNA, 48 bp, Artificial Sequence
  Synthetic sequence
accatcaacggagtgaccggctggcggctgtgcgaacgcattctggcg

SEQ ID NO: 71 - protein, 56 aa, Artificial Sequence
  Synthetic sequence
KGVKSVVNLVGEKLVYVQKWDGKETTYVREIKDGKLVVTLTMGDVVAVRSYRRATE

SEQ ID NO: 72 - DNA, 168 bp, Artificial Sequence
  Synthetic sequence
aaaggcgtgaagagcgtagtgaacttagttggagaaaaactggtctatgttcagaaatgggacggtaaggagaccacttatgtgcgagaaattaaggacggaaagcttgtggtgacgctgactatgggagacgtcgtagcagtgcggagttatagacgcgcgaccgaa

SEQ ID NO: 73 - protein, 117 aa, Artificial Sequence
  Synthetic sequence
KIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 74 - DNA, 351 bp, Artificial Sequence
  Synthetic sequence
aagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcg

SEQ ID NO: 75 - protein, 139 aa, Artificial Sequence
  Synthetic sequence
MVEKFVGTWKIADSHNFGEYLKAIGAPKELSDGGDATTPTLYISQKDGDKMTVKIENGPPTFLDTQVKFKLGEEFDEFPSDRRKGVKSVVNLVGEKLVYVQKWDGKETTYVREIKDGKLVVTLTMGDVVAVRSYRRATE

SEQ ID NO: 76 - DNA, 420 bp, Artificial Sequence
  Synthetic sequence
atggttgagaagtttgttggcacttggaagatagctgatagtcataactttggagaatacctgaaggccattggtgcaccgaaagaactttctgatggcggagacgcaacaactcccacactctatatcagccaaaaggacggggacaaaatgacagtaaagatcgaaaacggaccacccactttcctggatacgcaggtgaagtttaagctcggagaggagttcgatgaatttcccagcgaccgccggaaaggcgtgaagagcgtagtgaacttagttggagaaaaactggtctatgttcagaaatgggacggtaaggagaccacttatgtgcgagaaattaaggacggaaagcttgtggtgacgctgactatgggagacgtcgtagcagtgcggagttatagacgcgcgaccgaataa